(19) United States
(12) Patent Application Publication
(10) Pub. No.: US 2017/0095508 A1
(43) Pub. Date: Apr. 6, 2017

(54) ANTIMICROBIAL CLAY COMPOSITIONS AND METHODS OF USING

(71) Applicant: NUTRIQUEST, LLC, Mason City, IA (US)

(72) Inventors: Chad Hagen, Mankato, MN (US); Robert Musser, Mankato, MN (US); Ryan Cooney, Mankato, MN (US); Kim Friesen, Mankato, MN (US); Ran Song, Mankato, MN (US); Chester Wiernusz, Canton, GA (US)

(21) Appl. No.: 15/266,570

(22) Filed: Sep. 15, 2016

Related U.S. Application Data
(60) Provisional application No. 62/218,941, filed on Sep. 15, 2015.
(60) Provisional application No. 62/235,106, filed on Sep. 30, 2015.
(60) Provisional application No. 62/343,070, filed on May 30, 2016.

(51) Int. Cl. A61K 33/26; A01N 59/16; A61K 9/00; A23K 20/28; A01N 25/12; A61K 9/14; A23K 50/30; A23K 50/75; A01N 59/06; A61K 33/06

(52) U.S. Cl. CPC A61K 33/26; A01N 59/06; A01N 59/16; A61K 9/0056; A61K 33/06; A01N 25/12; A61K 9/14; A23K 50/30; A23K 50/75; A23K 20/28

(57) ABSTRACT

The present invention provides antimicrobial feed supplement compositions comprising clay, and methods of treating microbial infections in an animal using the antimicrobial compositions.

### CROSS-REFERENCE TO RELATED APPLICATION

This application relates to and claims the priority of U.S. Provisional Patent Application Ser. No. 62/218,941, which was filed Sep. 15, 2015, U.S. Provisional Patent Application Ser. No. 62/235,106, which was filed Sep. 30, 2015, and U.S. Provisional Patent Application Ser. No. 62/343,070, which was filed May 30, 2016, each of which is hereby incorporated by reference in its entirety.

### FIELD OF THE INVENTION

The present invention generally relates to methods of using antimicrobial clay, formulations comprising antimicrobial clay, and methods of treating microbes in an animal or in an animal environment using the antimicrobial clay and antimicrobial clay formulations.

### BACKGROUND OF THE INVENTION

In addition to controlling bacterial infections in animals and humans, antibiotics are extensively used to control bacterial contamination in some industrial processes, including fermentation, and to increase efficiency and growth rate of farm animals. As the use of conventional antibiotics increases for controlling bacteria for medical, veterinary, and agricultural purposes, or in other fields such as fermentation, the increasing emergence of antibiotic-resistant strains of pathogenic bacteria is an unwelcome consequence. As a result, public opinion and public policy has been increasingly calling for restricting the use of antibiotics as a general antibacterial. In fact, regulatory bodies such as the U.S. Food and Drug Administration have banned the use of human-class antibiotics in food-related industries. Additionally, antibiotic resistance is reducing the effectiveness of some antibiotics used to fight bacterial infections in humans. The evolution of resistant strains of bacteria is a natural phenomenon that occurs when bacteria are exposed to antibiotics, and resistant traits can be exchanged between certain types of bacteria.

Drug resistance of bacterial pathogens is presently one of the major causes of failure in the treatment of infectious diseases. The continued development of resistance to and against feed-grade antibiotics, however, has caused a setback in infectious disease prevention and control. Further, the use of feed-grade antibiotics, including virginiamycin and bacitracin, is being challenged due to the increasing public awareness of the negative impacts of antibiotic use and its effect on the environment and human health. One of the most significant problems associated with the reduction and elimination of antibiotics for use in ruminants, poultry, and swine will be the increase in incidences of diseases and decrease in productivity. A decrease in the use of antibiotics will also result in a decrease in the safety of the food that is consumed by humans as animal food products.

In the field of fermentation of sugar- or starch-containing feedstocks for the production of alcohol and alcoholic beverages, traditional methods of controlling bacterial contamination during fermentation have proven less than satisfactory. For instance, no antibiotics have proven to be effective for long-term control of bacterial contamination. Additionally, antibiotics carry through the fermentation and distillation process and end up in the distillers grains (DGs). The DGs provide a valuable feed product but with trace antibiotics, many farmers are reluctant to use DGs or must ration the DGs in the animal feed for the same reasons described above. Trace antibiotics in the DGs can cause bacteria in cows to mutate to an antibiotic-resistant strain. The U.S. Food and Drug Administration is currently considering banning the use of antibiotics in ethanol production due to the carryover of trace amounts of antibiotics.

With the decrease in effective antimicrobial treatments due to the emergence of resistant organisms, new antimicrobial therapeutics that are complementary and alternative to antibiotics are needed. The number of new antimicrobial therapies developed and approved has steadily decreased in the past three decades, leaving even fewer options to treat resistant organisms. For the foregoing reasons, a need exists for cost-effective methods of controlling pathogenic bacteria, including drug-resistant bacteria, that are flexible in range and that cannot be overcome by the bacteria by a single or small number of mutations.

### SUMMARY OF THE INVENTION

In one aspect, a method for controlling microbes is provided. The method comprises contacting the microbes with an antimicrobial effective amount of an antimicrobial clay, wherein the clay is mined clay. The antimicrobial clay may be clay mined in the Crater Lake region of the Cascade Mountains of Oregon. The antimicrobial clay may comprise an antimicrobial effective amount of a reducing agent. The antimicrobial clay may comprise an antimicrobial effective amount of aluminum. The antimicrobial clay may comprise about 1% to about 15% aluminum, or about 2% to about 5% aluminum. The antimicrobial clay may also comprise about 3% to about 10% pyrite. The antimicrobial clay may also comprise about 1% to about 5% Fe³⁺. The antimicrobial clay may also comprise about 3% to about 10% pyrite, and about 1% to about 5% Fe³⁺. The antimicrobial clay may comprise about 3% to about 10% pyrite, about 1% to about 5% Fe³⁺, and about 3% to 15% aluminum.

The antimicrobial clay may be mined. Alternatively, the antimicrobial clay may be naturally mined, and the level of reducing agent in the clay is adjusted to provide antimicrobial effective amounts of the reducing agent.

The average particle size of the antimicrobial clay may be less than about 500 microns in diameter, less than about 300 microns in diameter, between about 20 microns and about 200 microns in diameter, or between about 25 microns and about 150 microns in diameter.

The method may comprise administering the antimicrobial clay to an animal to inhibit the growth of bacteria. The bacteria may be selected from the group consisting of Clostridium perfringens, Aeromonas hydrophila, Yersinia enterocolitica, Vibrio spp., Leptospira spp., Mycobacterium ulcerans, Listeria spp., pathogenic strains of E. coli, Pseudomonas spp., Staphylococcus spp., Streptococcus sp., Clostridia, M. marinum, Lawsonia, Salmonella, Campylobacter, Enterococcus, and Liver abscess bacteria. The antimicrobial clay may be administered orally.

In some embodiments, the antimicrobial clay is formulated in a feed composition for oral administration to the animal. The amount of antimicrobial clay in a feed composition may range from about 0.1% to about 0.5% of the feed composition. The blue antimicrobial clay may be administered at a rate of about 3 to about 10 grams per animal per day or at a rate of at a rate of about 0.05 to about 5 grams/lb body weight/day. The red antimicrobial clay may be administered at a rate of about 0.3 to about 4 grams per animal per day or at a rate of at a rate of about 0.05 to about 5 grams/lb body weight/day.

In some embodiments, the antimicrobial clay is administered to a pig to control enterotoxigenic E. coli in the pig. In other embodiments, the antimicrobial clay is administered to a chicken to control necrotic enteritis in the chicken. In yet other embodiments, the antimicrobial clay is administered to a pig to control influenza in the pig. In other embodiments, the antimicrobial clay is administered to a pig to control scouring in the pig. In additional embodiments, the antimicrobial clay is administered to an animal to improve growth performance of the animal. The antimicrobial clay may be administered at least once daily.

In some embodiments, the method comprises contacting an animal's environment with the antibacterial clay to control pathogenic microbes in the animal's environment. In other embodiments, the method comprises contacting a fermenting mixture with the antimicrobial clay to control bacteria during fermentation.

In another aspect, a method for treating a microbial infection in an animal is provided. The method comprises administering a feed composition to the animal, wherein the composition comprises an antimicrobial effective amount of a mined antimicrobial clay. The antimicrobial clay is mined in the Crater Lake region of the Cascade Mountains of Oregon. The amount of antimicrobial clay in a feed composition ranges from about 0.05% to about 0.15%. The composition may be administered at least once daily. In some embodiments, the microbial infection is selected from enterotoxigenic E. coli in the pig, necrotic enteritis in the chicken, influenza in the pig, or scouring in the pig.

In yet another aspect, a method for improving growth performance of an animal is provided. The method comprises administering a feed composition to the animal, wherein the composition comprises an antimicrobial effective amount of an antimicrobial clay. The antimicrobial clay is mined in the Crater Lake region of the Cascade Mountains of Oregon. The amount of antimicrobial clay in a feed composition ranges from about 0.05% to about 0.15%. The composition may be administered at least once daily.

In an additional aspect, a method for controlling pathogenic microbes in an animal's environment is provided. The method comprises contacting the animal's environment with an antimicrobial clay. The antimicrobial clay is mined in the Crater Lake region of the Cascade Mountains of Oregon.

In another aspect, a method for controlling bacteria during fermentation is provided. The method comprises contacting a fermentation mixture with an antimicrobial clay. The antimicrobial clay is mined in the Crater Lake region of the Cascade Mountains of Oregon.

In yet another aspect, an antimicrobial feed composition is provided. The antimicrobial feed composition comprises an antimicrobial effective amount of an antimicrobial clay, wherein the clay is mined clay. The antimicrobial clay may be clay mined in the Crater Lake region of the Cascade Mountains of Oregon. The amount of antimicrobial clay in a feed composition may range from about 0.1% to about 0.5%. The antimicrobial clay may comprise about 1% to about 15% aluminum, about 2% to about 5% aluminum, about 3% to about 10% pyrite, about 1% to about 5% Fe3+, or combinations thereof. The antimicrobial clay may comprise about 3% to about 10% pyrite, and about 1% to about 5% Fe3+. The antimicrobial clay may also comprise about 3% to about 10% pyrite, about 1% to about 5% Fe3+, and about 3% to about 15% aluminum.

In another aspect, a method of treating a microbial infection in an animal is provided. The method comprises providing an antimicrobial clay, wherein the clay is mined clay, combining an antimicrobial effective amount of the antimicrobial clay with an animal feed composition to prepare an antimicrobial feed composition, and feeding the antimicrobial feed composition to the animal to treat the microbial infection. The antimicrobial clay may be clay mined in the Crater Lake region of the Cascade Mountains of Oregon. The antimicrobial effective amount of antimicrobial clay may be combined with the animal feed at the rate of about 0.1% to about 0.5% wt/wt of the antimicrobial feed composition. The antimicrobial clay may comprise about 3% to about 10% pyrite, and about 1% to about 5% Fe3+. The antimicrobial clay may comprise about 3% to about 10% pyrite, about 1% to about 5% Fe3+, and about 3% to about 15% aluminum.

In an additional aspect, a method of improving growth performance of an animal is provided. The method comprises providing an antimicrobial clay, wherein the clay is mined clay, combining an antimicrobial effective amount of the antimicrobial clay with an animal feed composition to prepare an antimicrobial feed composition, and feeding the antimicrobial feed composition to the animal to improve growth performance of the animal. The antimicrobial clay may be clay mined in the Crater Lake region of the Cascade Mountains of Oregon. The antimicrobial effective amount of antimicrobial clay may be combined with the animal feed at the rate of about 0.1% to about 0.5% wt/wt of the antimicrobial feed composition. The antimicrobial clay may comprise about 3% to about 10% pyrite, and about 1% to about 5% Fe3+. The antimicrobial clay may comprise about 3% to about 10% pyrite, about 1% to about 5% Fe3+, and about 3% to about 15% aluminum. The antimicrobial feed composition may be fed to the animal at least once daily.

### BRIEF DESCRIPTION OF DRAWINGS

The application file contains at least one photograph executed in color. Copies of this patent application publication with color photographs will be provided by the Office upon request and payment of the necessary fee.

The following drawings form part of the present disclosure and are included to further demonstrate certain aspects of the present disclosure. The disclosure may be better understood by reference to one or more of these drawings in combination with the detailed description of specific aspects presented herein.

FIG. 1A depicts a bar chart showing the average daily gain (ADG) of weanling pigs. NC=pigs not challenged with enterotoxigenic E. coli K88+ (ETEC) and not treated with Product V (PV). CON=control pigs challenged with ETEC but not treated with PV. PROD=pigs challenged with ETEC and treated with PV. a,b Means without a common superscript differ (P<0.05). c,d Means without a common superscript tend to differ (P<0.10).

FIG. 1B depicts a bar chart showing the average daily feed intake (ADFI) of weanling pigs. NC=pigs not challenged with ETEC and not treated with PV. CON=control pigs challenged with ETEC but not treated with PV. PROD=pigs challenged with ETEC and treated with PV. a,b Means without a common superscript differ (P<0.05). c,d Means without a common superscript tend to differ (P<0.10).

FIG. 1C depicts a bar chart showing the final body weight (BW) of weanling pigs. NC=pigs not challenged with ETEC and not treated with PV. CON=control pigs challenged with ETEC but not treated with PV. PROD=pigs challenged with ETEC and treated with PV. a,b Means without a common superscript differ (P<0.05). c,d Means without a common superscript tend to differ (P<0.10).

FIG. 2 depicts a bar chart showing the mortality of pigs at 24, 48, and 72 hrs post-challenge. NC=pigs not challenged with ETEC and not treated with PV. CON=control pigs challenged with ETEC but not treated with PV. PROD=pigs challenged with ETEC and treated with PV.

FIG. 3 depicts a bar chart showing the fecal consistency scores at 8, 24, 48, and 72 hrs post-challenge and average fecal consistency scores of weanling pigs. NC=pigs not challenged with ETEC and not treated with PV. CON=control pigs challenged with ETEC but not treated with PV. PROD=pigs challenged with ETEC and treated with PV. a,b,c Means without a common superscript differ (P<0.05).

FIG. 4A depicts a bar chart showing the total coliform counts. NC=pigs not challenged with ETEC and not treated with PV. CON=control pigs challenged with ETEC but not treated with PV. PROD=pigs challenged with ETEC and treated with PV. a,b,c Means without a common superscript differ (P<0.05).

FIG. 4B depicts a bar chart showing E. coli K88+ counts. NC=pigs not challenged with ETEC and not treated with PV. CON=control pigs challenged with ETEC but not treated with PV. PROD=pigs challenged with ETEC and treated with PV. a,b,c Means without a common superscript differ (P<0.05).

FIG. 4C depicts a bar chart showing pH of gastrointestinal digesta in weanling pigs. NC=pigs not challenged with ETEC and not treated with PV. CON=control pigs challenged with ETEC but not treated with PV. PROD=pigs challenged with ETEC and treated with PV. a,b,c Means without a common superscript differ (P<0.05).

FIG. 5A depicts a light microscope image of the general features of a pig ileum, including the number of follicles, the follicle area, and the submucosal thickness of the ileum.

FIG. 5B depicts a light microscope image of ileum from pig not challenged with ETEC.

FIG. 5C depicts a light microscope image of ileum from control pigs challenged with ETEC but not treated with PV.

FIG. 5D depicts a light microscope image of ileum from pig challenged with ETEC and treated with PV.

FIG. 6A depicts a bar chart showing the number of follicles/field of view of a light microscope image of pig ileums. NC=pigs not challenged with ETEC and not treated with PV. CON=control pigs challenged with ETEC but not treated with PV. PROD=pigs challenged with ETEC and treated with PV. a,b Means without a common superscript differ (P<0.05).

FIG. 6B depicts a bar chart showing average area of follicles in light microscope image of pig ileums. NC=pigs not challenged with ETEC and not treated with PV. CON=control pigs challenged with ETEC but not treated with PV. PROD=pigs challenged with ETEC and treated with PV. a,b Means without a common superscript differ (P<0.05).

FIG. 6C depicts a bar chart showing submucosal thickness of pig ileum. NC=pigs not challenged with ETEC and not treated with PV. CON=control pigs challenged with ETEC but not treated with PV. PROD=pigs challenged with ETEC and treated with PV. a,b Means without a common superscript differ (P<0.05).

FIG. 7 depicts a bar chart showing the weights of small intestine, large intestine, the total weight of the gastrointestinal tract (GIT), the liver, and the spleen. NC=pigs not challenged with ETEC and not treated with PV. CON=control pigs challenged with ETEC but not treated with PV. PROD=pigs challenged with ETEC and treated with PV. c, d Means without a common superscript tend to differ (P<0.10).

FIG. 8A depicts a bar chart showing necrotic enteritis-related mortality. NC=birds not challenged with Clostridium perfringens and not treated with PV. PV_0=birds challenged with Clostridium perfringens but not treated with PV. PV_1=birds challenged with Clostridium perfringens and treated with PV at 1 lb/ton. PV_2=birds challenged with Clostridium perfringens and treated with PV at 2 lb/ton. PV_3=birds challenged with Clostridium perfringens and treated with PV at 3 lb/ton. a,b,c Means without a common superscript differ (P<0.05).

FIG. 8B depicts a bar chart showing necrotic enteritis lesion score on day 21. NC=birds not challenged with Clostridium perfringens and not treated with PV. PV_0=birds challenged with Clostridium perfringens but not treated with PV. PV_1=birds challenged with Clostridium perfringens and treated with PV at 1 lb/ton. PV_2=birds challenged with Clostridium perfringens and treated with PV at 2 lb/ton. PV_3=birds challenged with Clostridium perfringens and treated with PV at 3 lb/ton. a,b,c Means without a common superscript differ (P<0.05).

FIG. 9 depicts a bar chart showing the cumulative body weight gain per cage and body weight gain at the following intervals: days 0 and 14 (D0-D14), days 14 and 21 (D14-D21), days 21 and 28 (D21-D28). NC=birds not challenged with Clostridium perfringens and not treated with PV. PV_0=birds challenged with Clostridium perfringens but not treated with PV. PV_1=birds challenged with Clostridium perfringens and treated with PV at 1 lb/ton. PV_2=birds challenged with Clostridium perfringens and treated with PV at 2 lb/ton. PV_3=birds challenged with Clostridium perfringens and treated with PV at 3 lb/ton. a,b Means without a common superscript differ (P<0.05).

FIG. 10A depicts a bar chart showing the body weight of unchallenged birds (red dotted horizontal arrow), and the body weight per cage of treated birds at day 14 (BW D14), day 21 (BW D21), and day 28 (BW D28). NC=birds not challenged with Clostridium perfringens and not treated with PV. PV_0=birds challenged with Clostridium perfringens but not treated with PV. PV_1=birds challenged with Clostridium perfringens and treated with PV at 1 lb/ton. PV_2=birds challenged with Clostridium perfringens and treated with PV at 2 lb/ton. PV_3=birds challenged with Clostridium perfringens and treated with PV at 3 lb/ton. a,b Means without a common superscript differ (P<0.05).

FIG. 10B depicts a bar chart showing the actual body weight per cage on day 28, and the number of birds/cage on day 28 (numbers in blue ovals). NC=birds not challenged with Clostridium perfringens and not treated with PV. PV_0=birds challenged with Clostridium perfringens but not treated with PV. PV_1=birds challenged with Clostridium perfringens and treated with PV at 1 lb/ton. PV_2=birds challenged with Clostridium perfringens and treated with PV at 2 lb/ton. PV_3=birds challenged with Clostridium perfringens and treated with PV at 3 lb/ton. a,b Means without a common superscript differ (P<0.05).

FIG. 11A depicts a bar chart showing feed conversion ratio at days 0-14 (D0-D14). NC=birds not challenged with Clostridium perfringens and not treated with PV. PV_0=birds challenged with Clostridium perfringens but not treated with PV. PV_1=birds challenged with Clostridium perfringens and treated with PV at 1 lb/ton. PV_2=birds challenged with Clostridium perfringens and treated with PV at 2 lb/ton. PV_3=birds challenged with Clostridium perfringens and treated with PV at 3 lb/ton. a,b,c Means without a common superscript differ (P<0.05).

FIG. 11B depicts a bar chart showing feed conversion ratio at days 14-28 (D14-D28). NC=birds not challenged with Clostridium perfringens and not treated with PV. PV_0=birds challenged with Clostridium perfringens but not treated with PV. PV_1=birds challenged with Clostridium perfringens and treated with PV at 1 lb/ton. PV_2=birds challenged with Clostridium perfringens and treated with PV at 2 lb/ton. PV_3=birds challenged with Clostridium perfringens and treated with PV at 3 lb/ton. a,b,c Means without a common superscript differ (P<0.05).

FIG. 11C depicts a bar chart showing feed conversion ratio at days 14-21 (D14-D21). NC=birds not challenged with Clostridium perfringens and not treated with PV. PV_0=birds challenged with Clostridium perfringens but not treated with PV. PV_1=birds challenged with Clostridium perfringens and treated with PV at 1 lb/ton. PV_2=birds challenged with Clostridium perfringens and treated with PV at 2 lb/ton. PV_3=birds challenged with Clostridium perfringens and treated with PV at 3 lb/ton. a,b,c Means without a common superscript differ (P<0.05).

FIG. 11D depicts a bar chart showing feed conversion ratio at days 21-28 (D21-D28). NC=birds not challenged with Clostridium perfringens and not treated with PV. PV_0=birds challenged with Clostridium perfringens but not treated with PV. PV_1=birds challenged with Clostridium perfringens and treated with PV at 1 lb/ton. PV_2=birds challenged with Clostridium perfringens and treated with PV at 2 lb/ton. PV_3=birds challenged with Clostridium perfringens and treated with PV at 3 lb/ton. a,b,c Means without a common superscript differ (P<0.05).

FIG. 12A depicts a chart showing the ADG during phase 1 (day 0 to day 7). CON=pigs not treated with Evosure Core or PV. EC=pigs treated with 1.0 lb/ton Evosure Core. V=pigs treated with 2.0 lb/ton PV. EC/V=pigs treated with 1.0 lb/ton Evosure Core and 2.0 lb/ton PV.

FIG. 12B depicts a chart showing the ADFI during phase 1 (day 0 to day 7). CON=pigs not treated with Evosure Core or PV. EC=pigs treated with 1.0 lb/ton Evosure Core. V=pigs treated with 2.0 lb/ton PV. EC/V=pigs treated with 1.0 lb/ton Evosure Core and 2.0 lb/ton PV.

FIG. 12C depicts a chart showing the F:G ratio of pigs during phase 1 (day 0 to day 7). CON=pigs not treated with Evosure Core or PV. EC=pigs treated with 1.0 lb/ton Evosure Core. V=pigs treated with 2.0 lb/ton PV. EC/V=pigs treated with 1.0 lb/ton Evosure Core and 2.0 lb/ton PV.

FIG. 13A depicts a chart showing the ADG during phase 2 (day 7 to day 22). CON=pigs not treated with Evosure Core or PV. EC=pigs treated with 1.0 lb/ton Evosure Core. V=pigs treated with 2.0 lb/ton PV. EC/V=pigs treated with 1.0 lb/ton Evosure Core and 2.0 lb/ton PV. a,b Means without a common superscript differ (P<0.05).

FIG. 13B depicts a chart showing the ADFI during phase 2 (day 7 to day 22). CON=pigs not treated with Evosure Core or PV. EC=pigs treated with 1.0 lb/ton Evosure Core. V=pigs treated with 2.0 lb/ton PV. EC/V=pigs treated with 1.0 lb/ton Evosure Core and 2.0 lb/ton PV. a,b Means without a common superscript differ (P<0.05).

FIG. 13C depicts a chart showing the F:G ratio of pigs during phase 2 (day 7 to day 22). CON=pigs not treated with Evosure Core or PV. EC=pigs treated with 1.0 lb/ton Evosure Core. V=pigs treated with 2.0 lb/ton PV. EC/V=pigs treated with 1.0 lb/ton Evosure Core and 2.0 lb/ton PV. a,b Means without a common superscript differ (P<0.05).

FIG. 14A depicts a chart showing the ADG of pigs during phase 3 (day 22 to day 33). CON=pigs not treated with Evosure Core or PV. EC=pigs treated with 1.0 lb/ton Evosure Core. V=pigs treated with 2.0 lb/ton PV. EC/V=pigs treated with 1.0 lb/ton Evosure Core and 2.0 lb/ton PV.

FIG. 14B depicts a chart showing the ADFI of pigs during phase 3 (day 22 to day 33). CON=pigs not treated with Evosure Core or PV. EC=pigs treated with 1.0 lb/ton Evosure Core. V=pigs treated with 2.0 lb/ton PV. EC/V=pigs treated with 1.0 lb/ton Evosure Core and 2.0 lb/ton PV.

FIG. 14C depicts a chart showing the F:G ratio of pigs during phase 3 (day 22 to day 33). CON=pigs not treated with Evosure Core or PV. EC=pigs treated with 1.0 lb/ton Evosure Core. V=pigs treated with 2.0 lb/ton PV. EC/V=pigs treated with 1.0 lb/ton Evosure Core and 2.0 lb/ton PV.

FIG. 15A depicts a chart showing the ADG of pigs during the entire period of the study (day 0 to day 33). CON=pigs not treated with Evosure Core or PV. EC=pigs treated with 1.0 lb/ton Evosure Core. V=pigs treated with 2.0 lb/ton PV. EC/V=pigs treated with 1.0 lb/ton Evosure Core and 2.0 lb/ton PV. a,b Means without a common superscript differ (P<0.05).

FIG. 15B depicts a chart showing the ADFI of pigs during the entire period of the study (day 0 to day 33). CON=pigs not treated with Evosure Core or PV. EC=pigs treated with 1.0 lb/ton Evosure Core. V=pigs treated with 2.0 lb/ton PV. EC/V=pigs treated with 1.0 lb/ton Evosure Core and 2.0 lb/ton PV. a,b Means without a common superscript differ (P<0.05).

FIG. 15C depicts a chart showing the F:G ratio of pigs during the entire period of the study (day 0 to day 33). CON=pigs not treated with Evosure Core or PV. EC=pigs treated with 1.0 lb/ton Evosure Core. V=pigs treated with 2.0 lb/ton PV. EC/V=pigs treated with 1.0 lb/ton Evosure Core and 2.0 lb/ton PV. a,b Means without a common superscript differ (P<0.05).

FIG. 16A depicts a chart showing the body weight of pigs at the end of phase 1. CON=pigs not treated with Evosure Core or PV. EC=pigs treated with 1.0 lb/ton Evosure Core. V=pigs treated with 2.0 lb/ton PV. EC/V=pigs treated with 1.0 lb/ton Evosure Core and 2.0 lb/ton PV. a,b Means without a common superscript differ (P<0.05).

FIG. 16B depicts a chart showing the body weight of pigs at the end of phase 2. CON=pigs not treated with Evosure Core or PV. EC=pigs treated with 1.0 lb/ton Evosure Core. V=pigs treated with 2.0 lb/ton PV. EC/V=pigs treated with 1.0 lb/ton Evosure Core and 2.0 lb/ton PV. a,b Means without a common superscript differ (P<0.05).

FIG. 16C depicts a chart showing the body weight of pigs at the end of phase 3. CON=pigs not treated with Evosure Core or PV. EC=pigs treated with 1.0 lb/ton Evosure Core. V=pigs treated with 2.0 lb/ton PV. EC/V=pigs treated with 1.0 lb/ton Evosure Core and 2.0 lb/ton PV. a,b Means without a common superscript differ (P<0.05).

FIG. 17 depicts a chart showing the removal rate of pigs during phase 1 of the study (day 0 to day 11). CON/low Zn=pigs administered 110 ppm Zn. CON/high Zn=pigs administered 3000 ppm Zn. PV/low Zn=pigs administered 110 ppm Zn and 2.0 lb/ton PV. PV/high Zn=pigs administered 3000 ppm Zn and 2.0 lb/ton PV.

FIG. 18A depicts a chart showing the ADG during phase 1 (day 0 to day 11). CON/low Zn=pigs administered 110 ppm Zn. CON/high Zn=pigs administered 3000 ppm Zn. PV/low Zn=pigs administered 110 ppm Zn and 2.0 lb/ton PV. PV/high Zn=pigs administered 3000 ppm Zn and 2.0 lb/ton PV. a,b Means without a common superscript differ (P<0.05).

FIG. 18B depicts a chart showing the ADFI during phase 1 (day 0 to day 11). CON/low Zn=pigs administered 110 ppm Zn. CON/high Zn=pigs administered 3000 ppm Zn. PV/low Zn=pigs administered 110 ppm Zn and 2.0 lb/ton PV. PV/high Zn=pigs administered 3000 ppm Zn and 2.0 lb/ton PV. a,b Means without a common superscript differ (P<0.05).

FIG. 18C depicts a chart showing the F:G ratio of pigs during phase 1 (day 0 to day 11). CON/low Zn=pigs administered 110 ppm Zn. CON/high Zn=pigs administered 3000 ppm Zn. PV/low Zn=pigs administered 110 ppm Zn and 2.0 lb/ton PV. PV/high Zn=pigs administered 3000 ppm Zn and 2.0 lb/ton PV. a,b Means without a common superscript differ (P<0.05).

FIG. 19A depicts a chart showing the ADG during phase 2 (day 11 to day 26). CON/low Zn=pigs administered 110 ppm Zn. CON/high Zn=pigs administered 3000 ppm Zn. PV/low Zn=pigs administered 110 ppm Zn and 2.0 lb/ton PV. PV/high Zn=pigs administered 3000 ppm Zn and 2.0 lb/ton PV.

FIG. 19B depicts a chart showing the ADFI during phase 2 (day 11 to day 26). CON/low Zn=pigs administered 110 ppm Zn. CON/high Zn=pigs administered 3000 ppm Zn. PV/low Zn=pigs administered 110 ppm Zn and 2.0 lb/ton PV. PV/high Zn=pigs administered 3000 ppm Zn and 2.0 lb/ton PV.

FIG. 19C depicts a chart showing the F:G ratio of pigs during phase 2 (day 11 to day 26). CON/low Zn=pigs administered 110 ppm Zn. CON/high Zn=pigs administered 3000 ppm Zn. PV/low Zn=pigs administered 110 ppm Zn and 2.0 lb/ton PV. PV/high Zn=pigs administered 3000 ppm Zn and 2.0 lb/ton PV.

FIG. 20A depicts a chart showing the ADG of pigs during the overall duration of the study (day 0 to day 26). CON/low Zn=pigs administered 110 ppm Zn. CON/high Zn=pigs administered 3000 ppm Zn. PV/low Zn=pigs administered 110 ppm Zn and 2.0 lb/ton PV. PV/high Zn=pigs administered 3000 ppm Zn and 2.0 lb/ton PV.

FIG. 20B depicts a chart showing the ADFI of pigs during the overall duration of the study (day 0 to day 26). CON/low Zn=pigs administered 110 ppm Zn. CON/high Zn=pigs administered 3000 ppm Zn. PV/low Zn=pigs administered 110 ppm Zn and 2.0 lb/ton PV. PV/high Zn=pigs administered 3000 ppm Zn and 2.0 lb/ton PV.

FIG. 20C depicts a chart showing the F:G ratio of pigs during the overall duration of the study (day 0 to day 26). CON/low Zn=pigs administered 110 ppm Zn. CON/high Zn=pigs administered 3000 ppm Zn. PV/low Zn=pigs administered 110 ppm Zn and 2.0 lb/ton PV. PV/high Zn=pigs administered 3000 ppm Zn and 2.0 lb/ton PV.

FIG. 21A depicts a chart showing the initial body weight of pigs. CON/low Zn=pigs administered 110 ppm Zn. CON/high Zn=pigs administered 3000 ppm Zn. PV/low Zn=pigs administered 110 ppm Zn and 2.0 lb/ton PV. PV/high Zn=pigs administered 3000 ppm Zn and 2.0 lb/ton PV.

FIG. 21B depicts a chart showing the body weight of pigs at the end of phase 1 (day 0 to day 11). CON/low Zn=pigs administered 110 ppm Zn. CON/high Zn=pigs administered 3000 ppm Zn. PV/low Zn=pigs administered 110 ppm Zn and 2.0 lb/ton PV. PV/high Zn=pigs administered 3000 ppm Zn and 2.0 lb/ton PV.

FIG. 21C depicts a chart showing the body weight of pigs at the end of phase 2 (day 11 to day 26). CON/low Zn=pigs administered 110 ppm Zn. CON/high Zn=pigs administered 3000 ppm Zn. PV/low Zn=pigs administered 110 ppm Zn and 2.0 lb/ton PV. PV/high Zn=pigs administered 3000 ppm Zn and 2.0 lb/ton PV.

FIG. 22A depicts a chart showing the change in pH over a 48 hour time course for the TP25 group with initial pH of 5.5 and 6

FIG. 22B depicts a chart showing the change in pH over a 48 hour time course for the TP50 group with initial pH of 5.5 and 6.

FIG. 22C depicts a chart showing the change in pH over a 48 hour time course for the TP75 group with initial pH of 5.5 and 6.

FIG. 22D depicts a chart showing the change in pH over a 48 hour time course for the Blank group with initial pH of 5.5 and 6.

FIG. 23A depicts a chart showing the change in dry matter disappearance (DMD) in an in vitro ruminal bag study with different dosages of test product (TP) and blank controls over a 48 hour time course for the TP25 group with initial DMD of 5.5 and 6.

FIG. 23B depicts a chart showing the change in DMD over a 48 hour time course for the TP50 group with initial DMD of 5.5 and 6.

FIG. 23C depicts a chart showing the change in DMD over a 48 hour time course for the TP75 group with initial DMD of 5.5 and 6.

FIG. 23D depicts a chart showing the change in DMD over a 48 hour time course for the Blank group with initial DMD of 5.5 and 6.

FIG. 24A depicts a chart showing the pre-challenge ADG of weanling pigs.

FIG. 24B depicts a chart showing the post-challenge ADG of weanling pigs.

FIG. 25A depicts a chart showing the average fecal score and the fecal score at 72 hr post-challenge. 0=normal stool; 3=severe diarrhea. a,b Means without a common superscript differ (P<0.05).

FIG. 25B depicts a chart showing the frequency of diarrhea in the pigs. Frequency=diarrhea days/pig days×100, and diarrhea days=number of pig days with diarrhea score 2. c,d Means without a common superscript tend to differ (P<0.10).

FIG. 26A depicts a chart showing the total E. coli and E. coli F18 count in log cfu/g.

FIG. 26B depicts a chart showing the % pigs with undetectable E. coli F18.

### DETAILED DESCRIPTION OF THE INVENTION

The present invention is directed to methods of using antimicrobial clay. In particular, antimicrobial clay and methods of using the antimicrobial clay to control microbes have been discovered. The antimicrobial clay may be used to control microbes as an alternative and complementary treatment to antibiotics. The antimicrobial clay may be used to treat microbial infections in animals. For instance, the antimicrobial clay may be used to control microbial infections in animals when added as a dietary supplement to animal feed compositions or to an animal's drinking water. Additionally, the antimicrobial clay may be administered to animals to improve growth performance of the animal. The antimicrobial clay may also be used to control microbes when used in an animal's environment, or to control bacteria during fermentation.

### I. Antimicrobial Clay

In one aspect, the present disclosure provides antimicrobial clay. An antimicrobial clay may be used alone. Alternatively, an antimicrobial clay may be formulated with other ingredients to facilitate administration and effective use. For instance, the antimicrobial clay may be formulated with nutritive or other pharmaceutical agents for administration to an animal. The antimicrobial clay may also be dispersed in an animal's environment to control microbes. The clay and formulations comprising the antimicrobial clay are described below.

A. Clay

The term “clay” as used herein refers to a fine-grained natural rock or soil material that combines one or more clay minerals with traces of metal oxides and organic matter. Clays from natural geologic clay deposits are mostly composed of silicate minerals containing variable amounts of water trapped in the mineral structure. Additionally, as it will be recognized by an individual skilled in the art, a clay may further comprise various amounts of metal oxides, organic matter, and other materials that may be mixed in with the clay. Sometimes clays comprise varying amounts of iron, magnesium, alkali metals, alkaline earths and other cations. Depending on the content of the soil, clay can appear in various colors, from white to dull gray or brown to a deep orange-red. Clays may be broadly classified into swelling clays, non-swelling clays, and mixed layer clays.

A clay of the present disclosure has antimicrobial properties. An antimicrobial clay of the invention may be capable of controlling any one or more of bacteria, viruses, protozoans such as Cryptosporidium spp. and giardia, and fungi such as mold and mildew. As used herein, the term “antimicrobial” is used to indicate that antimicrobial clay may either kill microbes, and therefore be “microbicidal,” or prevents microbes from growing and reproducing while not necessarily killing them otherwise, and therefore be “biostatic.” Methods of determining if an agent, including clay, has antimicrobial properties are known in the art, and generally comprise contacting microbes with the agent in vivo or in vitro, and determining the effect of the agent on growth of the microbe. Preferably, an antimicrobial clay of the disclosure has antibacterial properties.

Any clay may be used in a composition or method of the present disclosure, provided the clay has antimicrobial properties. Without wishing to be bound by theory, the presence of an antimicrobial effective amount of one or more minerals, elements, or reducing agents in an antimicrobial clay of the present disclosure may improve the antimicrobial properties of the clay. As such, an antimicrobial clay of the present disclosure preferably comprises one or more minerals, elements, or reducing agents. Non-limiting examples of reducing agents that may be found in clays include iron-rich phases such as Fe-smectite, biotite, jarosite, pyrite, magnetite, hematite, goethite, amphibole, polymorphs of FeS₂, which include pyrite and marcasite, pyrrhotite, manganese oxides, FeS₂, FeS, FeSO4, and other minerals or compounds that contain soluble reducing transition metals with like properties. In addition, divalent iron within the structure of a clay mineral itself may also serve as a reducing agent. Preferably, an antimicrobial clay of the present disclosure comprises antimicrobial effective amounts of pyrite as one of the reducing agents. Pyrite has been implicated in spontaneous production of chemical radicals such as OH. and O²⁻ that may be highly damaging to biomolecules such as sugars, fatty acids or proteins located on bacterial cell surfaces and within cells. Additionally, the Fe²⁺ from pyrite may produce intracellular Fenton-type reactions. The reaction products could damage nucleic acids such as DNA or RNA or hamper cellular metabolic functions.

Also preferably, an antimicrobial clay of the present disclosure comprises antimicrobial effective amounts of soluble reducing compounds comprising transition metal ions as one of the reducing agents. In various embodiments, the transition metal ions may be chosen from scandium ions, yttrium ions, titanium ions, zirconium ions, halfium ions, vanadium ions, niobium ions, tantalum ions, chromium ions, molybdenum ions, tungsten ions, manganese ions, technetium ions, rhenium ions, iron ions, ruthenium ions, osmium ions, cobalt ions, rhodium ions, iridium ions, nickel ions, palladium ions, platinum ions, copper ions, silver ions, and gold ions. Generally, these transition metal ions may be in various oxidation states from +1 to +8. Non-limiting examples of suitable salts may include halides (fluoride, chloride, bromide, iodide), carbonates, hydrogen carbonates, carboxylates (such as acetates trifluoroacetate, propionates, butyrates, etc.), alkoxides, acetylacetonate, oxides, oxyhalides, sulfides, sulfites, hydrogensulfide, sulfates, hydrosulfates, phosphates, hydrogen phosphates, dihydrogenphosphates, pyrophosphate, borates, hydroxides, nitrates, nitrite, methanesulfonates, tosylates, triflates, hypochlorite, chlorite, chlorate, perchlorate, thiosulfate, oxalate, tartrate, cyanate, thiocyanate, and combinations thereof. Even more preferred, an antimicrobial clay of the present disclosure comprises antimicrobial effective amounts of soluble reducing compounds comprising iron ions as one of the reducing agents, particularly Fe3+.

The one or more reducing agents may be present in the clay at a level ranging from about 0.1% to about 30% (wt/wt) of the clay. For instance, the amount of reducing agents in a clay of the present disclosure may range from about 0.1% to about 5% (w/w), from about 5% to about 10%, from about 10% to about 15%, from about 15% to about 20%, from about 20% to about 25%, or from about 25% to about 30%. When the antimicrobial clay comprises pyrite as one of the reducing agents, the amount of pyrite in the clay of the present disclosure ranges from about 1% to about 15%, more preferably from about 3% to about 10%. When the antimicrobial clay comprises Fe³⁺ as one of the reducing agents, the amount of Fe³⁺ in the clay of the present disclosure ranges from about 1% to about 15%, more preferably from about 1% to about 5%.

Also preferably, an antimicrobial clay of the present disclosure comprises antimicrobial effective amounts of elements known to have antibacterial effects. Without wishing to be bound by theory, the presence of an antimicrobial effective amount of one or more minerals may promote cell toxicity through membrane damage during oxidation of the mineral. Non-limiting examples of elements known to have antibacterial effects that may be in an antimicrobial clay of the disclosure include aluminum, antimony, arsenic, barium, beryllium, bismuth, boron, cadmium, calcium, chromium, cobalt, copper, fluorine, gallium, germanium, gold, iron, lanthanum, lead, lithium, magnesium, manganese, mercury, molybdenum, nickel, niobium, phosphorus, potassium, rubidium, scandium, selenium, silver, sodium, strontium, tellurium, thallium, thorium, tin, titanium, tungsten, vanadium, yttrium, zinc, and zirconium.

Preferably, an antimicrobial clay of the invention comprises an antimicrobial effective amount of one or more of aluminum, barium, chromium, cobalt, gallium, iron, lanthanum, molybdenum, nickel, scandium, and yttrium. Even more preferred, an antimicrobial clay of the present disclosure comprises antimicrobial effective amounts of aluminum as one of the elements known to have antibacterial effects.

When an antimicrobial clay of the present disclosure comprises antimicrobial effective amounts of barium as an element known to have antibacterial effects, the antimicrobial clay may comprise about 30 to about 100 ppm barium, more preferably about 50 to about 80 ppm barium. When an antimicrobial clay of the present disclosure comprises antimicrobial effective amounts of chromium as an element known to have antibacterial effects, the antimicrobial clay may comprise about 1 to about 50 ppm chromium, more preferably about 5 to about 40 ppm chromium. When an antimicrobial clay of the present disclosure comprises antimicrobial effective amounts of cobalt as an element known to have antibacterial effects, the antimicrobial clay may comprise about 1 to about 20 ppm cobalt, more preferably about 3 to about 10 ppm cobalt. When an antimicrobial clay of the present disclosure comprises antimicrobial effective amounts of gallium as an element known to have antibacterial effects, the antimicrobial clay may comprise about 1 to about 50 ppm gallium, more preferably about 5 to about 15 ppm gallium. When an antimicrobial clay of the present disclosure comprises antimicrobial effective amounts of iron as an element known to have antibacterial effects, the antimicrobial clay may comprise about 0.1 to about 10% iron, more preferably about 1 to about 5% iron. When an antimicrobial clay of the present disclosure comprises antimicrobial effective amounts of lanthanum as an element known to have antibacterial effects, the antimicrobial clay may comprise about 10 to about 50 ppm lanthanum, more preferably about 15 to about 40 ppm lanthanum. When an antimicrobial clay of the present disclosure comprises antimicrobial effective amounts of molybdenum as an element known to have antibacterial effects, the antimicrobial clay may comprise about 0.01 to about 5 ppm molybdenum, more preferably about 0.05 to about 1 ppm molybdenum. When an antimicrobial clay of the present disclosure comprises antimicrobial effective amounts of nickel as an element known to have antibacterial effects, the antimicrobial clay may comprise about 1 to about 30 ppm nickel, more preferably about 2 to about 30 ppm nickel. When an antimicrobial clay of the present disclosure comprises antimicrobial effective amounts of scandium as an element known to have antibacterial effects, the antimicrobial clay may comprise about 1 to about 30 ppm scandium, more preferably about 5 to about 15 ppm scandium. When an antimicrobial clay of the present disclosure comprises antimicrobial effective amounts of yttrium as an element known to have antibacterial effects, the antimicrobial clay may comprise about 5 to about 50 ppm yttrium, more preferably about 15 to about 25 ppm yttrium.

Preferably, when an antimicrobial clay of the present disclosure comprises antimicrobial effective amounts of elements known to have antibacterial effects, the element is aluminum. When an antimicrobial clay of the present disclosure comprises antimicrobial effective amounts of aluminum as an element known to have antibacterial effects, the antimicrobial clay may comprise about 1 to about 15% aluminum, more preferably about 2 to about 5% aluminum.

An antimicrobial clay may be a swelling clay, a non-swelling clay, a mixed layer clay, or a combination of a swelling clay, a non-swelling clay, and a mixed layer clay. In some embodiments, an antimicrobial clay of the present disclosure is a swelling clay. Swelling or expansive clays are clays prone to large volume changes (swelling and shrinking) that are directly related to changes in water content. Swelling clays are generally referred to as smectite clays. Smectite clays have approximately 1-nm thick 2:1 layers (c-direction of unit cell) separated by hydrated interlayer cations which give rise to the clay's swelling. The “a” and “b” dimensions of the mineral are on the order of several microns. The layers themselves are composed of two opposing silicate sheets, which contain Si and Al in tetrahedral coordination with oxygen, separated by an octahedral sheet that contains Al, Fe and Mg in octahedral coordination with hydroxyls. The surfaces of the 2:1 layers (two tetrahedral sheets with an octahedral sheet in between) carry a net negative charge that is balanced by interlayer cations. The charged surfaces of the 2:1 layers attract cations and water, which leads to swelling.

Smectite clays may be classified with respect to the location of the negative charge on the 2:1 layers, and based on the composition of the octahedral sheet (either dioctahedral or trioctahedral). Dioctahedral smectites include beidellite having the majority of charge in the tetrahedral sheet, and montmorillonite having the majority of charge in the octahedral sheet. Similar trioctahedral smectites are saponite and hectorite. Swelling and other properties of smectite can be altered by exchanging the dominant interlayer cation. For example, swelling can be limited to 2 water layers by exchanging Na for Ca.

Smectite clays may be naturally mined. Alternatively, smectite clays may be synthesized. Methods of synthesizing smectite clays may be as described in U.S. Pat. No. 4,861,584, the disclosure of which is incorporated by reference herein in its entirety.

In other embodiments, an antimicrobial clay of the present disclosure is a non-swelling clay, also generally known as illite clays. Illite clays are similar in structure to smectite clays, but have their 2:1 layers bound together by poorly hydrated potassium ions, and for that reason do not swell.

In preferred embodiments, an antimicrobial clay of the present disclosure is a mixed-layer clay. Mixed-layer clays are generally referred to as rectorite and are composed of ordered mixed layers of illite and smectite. Layers of illite and smectite in rectorite clays may be random or regular. Ordering of illite and smectite layers in rectorite may be referred to as R⁰ ordered or R¹ ordered illite-smectite. R¹-ordered illite-smectite is ordered in an ISISIS fashion, whereas R0 describes random ordering. Other advanced ordering types may also be described. In exemplary embodiments, a clay of the present disclosure is a rectorite having R¹ ordered layers of illite and smectite.

Preferably, an antimicrobial clay of the present disclosure is a K-rectorite. More preferably, the antimicrobial clay is a K-rectorite comprising antimicrobial effective amounts of a reducing agent. Even more preferred, the antimicrobial clay is a K-rectorite comprising antimicrobial effective amounts of pyrite, or a K-rectorite comprising antimicrobial effective amounts of Fe³⁺.

An antimicrobial clay of the present disclosure may be an unrefined naturally occurring antimicrobial clay. Alternatively, an antimicrobial clay may be a refined antimicrobial clay purified from other material normally present in naturally occurring antimicrobial clay. Additionally, an antimicrobial clay may be purified to provide a substantially single form of the antimicrobial clay. For instance, when an antimicrobial clay is a rectorite clay, the clay may be purified to provide a substantially pure K-rectorite clay, a substantially pure Na-rectorite clay, or a substantially pure Ca-rectorite clay. In some embodiments, an antimicrobial clay is a naturally occurring antimicrobial clay. In other embodiments, an antimicrobial clay is a refined antimicrobial clay. In other embodiments, an antimicrobial clay is a purified antimicrobial clay.

In some embodiments, an antimicrobial clay is an unrefined, naturally occurring antimicrobial clay. In another embodiment, an antimicrobial clay is a refined naturally occurring antimicrobial clay. In yet other embodiments, an antimicrobial clay is synthesized. Methods of synthesizing antimicrobial clays may be as described in U.S. Patent Publication No. 2013/0004544, the disclosure of which is incorporated by reference herein in its entirety. In other embodiments, antimicrobial clays are naturally mined, and the levels of reducing agents in the mined clays are adjusted to provide antimicrobial effective amounts of reducing agents in the clay. Antimicrobial effective amounts of reducing agents may be as described above.

In exemplary embodiments, an antimicrobial clay of the present disclosure is a naturally mined antimicrobial clay supplied by Oregon Mineral Technologies (OMT), Grants Pass, Oreg., also known as blue clay. The source of the blue clay is an open pit mine in hydrothermally altered, pyroclastic material in the Cascade Mountains. The antibacterial activity of the blue clay supplied by OMT has been proven to completely eliminate Escherichia coli, Staphylococcus aureus, Pseudomonas aeruginosa, Salmonella typhimurium, and antibiotic resistant extended-spectrum beta lactamase (ESBL) E. coli and methicillin resistant S. aureus (MRSA) within 24 hrs. (see, for example, Cunningham et al. (2010) PLoS One 5(3): e9456; Williams et al. (2011) Environ Sci Technol 45(8):3768-3773; and U.S. Patent Publication No. 2013/0004544). Without wishing to be bound by theory, the antibacterial properties of the clay may be due to a rare antimicrobial transition metal combination, including a level of pyrite ranging from about 3% to about 10% wt/wt and/or a level of pyrite ranging from about 1% to about 5% wt/wt.

In other exemplary embodiments, an antimicrobial clay of the present disclosure is a natural red clay mined in the Cascade Mountain region of Oregon, more specifically a red clay mined in the crater lake region of the Cascade Mountains of Oregon. Without wishing to be bound by theory, the antibacterial properties of the red clay may be due to the presence of antimicrobial effective amounts of aluminum as described above, among other properties.

An antimicrobial clay may also be modified with various substituents to alter the properties of the clay. Non-limiting examples of modifications include modification with organic material, polymers, reducing agents, and various elements such as sodium, iron, silver, or bromide, or by treatment with a strong acid. In some embodiments, an antimicrobial clay of the present disclosure is modified with reducing metal oxides. In preferred alternatives of the embodiments, when an antimicrobial clay is modified with reducing metal oxides, the antimicrobial clay is modified with pyrite.

The particle size of the antimicrobial clay may be an important factor that can affect its effectiveness, as well as bioavailability, blend uniformity, segregation, and flow properties. In general, smaller particle sizes of clay increase its effectiveness by increasing the surface area. In various embodiments, the average particle size of the clay is less than about 500 microns in diameter, or less than about 450 microns in diameter, or less than about 400 microns in diameter, or less than about 350 microns in diameter, or less than about 300 microns in diameter, or less than about 250 microns in diameter, or less than about 200 microns in diameter, or less than about 150 microns in diameter, or less than about 100 microns in diameter, or less than about 75 microns in diameter, or less than about 50 microns in diameter, or less than about 25 microns in diameter, or less than about 15 microns in diameter. In some applications, the use of particles less than 15 microns in diameter may be advantageous. Preferably, the average particle size of the clay is about 1 to about 200 microns in diameter, preferably from about 10 to about 150 microns in diameter.

Similarly, in embodiments wherein a reducing agent may be added to an antimicrobial clay, the particle size of a reducing agent may also be an important factor that can affect its effectiveness, and in general, smaller particle sizes increase its effectiveness. Preferably, the average particle size of the reducing agent that may be added to an antimicrobial clay is less than 1 micron in size.

B. Dietary Supplements or Feed Compositions Comprising Antimicrobial Clay

One aspect of the present invention provides dietary supplements or feed compositions comprising a therapeutically effective amount of antimicrobial clay. A therapeutically effective amount of an antimicrobial clay in a feed supplement composition can and will vary depending on the antimicrobial clay, the body weight, sex, age and/or medical condition of the animal, the severity and extent of the infectious disease in the animal, the method of administration, the duration of treatment, as well as the species of the animal, and may be determined experimentally using methods known in the art.

Generally, the amount of an antimicrobial clay present in a feed or supplement composition will be at least 0.001% (w/w) of the total composition. In one embodiment, the amount of an antimicrobial clay in the composition ranges from about 0.001% to about 100% (w/w). For instance, the amount of an antimicrobial clay in the composition may range from about 0.001% to about 50% (w/w), from about 25% to about 75% (w/w), or about 50% to about 100% (w/w). Preferably, the amount of an antimicrobial clay in a feed or supplement composition ranges from between about 0.001% to about 15% (w/w), more preferably from about 0.1% to about 10% (w/w), and even more preferably from about 0.1% to about 0.5% (w/w).

The terms “feed”, “food”, “feed composition”, and “feed supplement”, are used herein interchangeably and may refer to any feed composition normally fed to an animal. Feed compositions normally fed to an animal are known in the art. A feed composition may include one or more components of an animal feed. Non-limiting examples of feed matter or animal feed matter may include, without limitation: corn or a component of corn, such as, for example, corn meal, corn fiber, corn hulls, corn DDGS (distiller's dried grain with solubles), silage, ground corn, corn germ, corn gluten, corn oil, or any other portion of a corn plant; soy or a component of soy, such as, for example, soy oil, soy meal, soy hulls, soy silage, ground soy, or any other portion of a soy plant; wheat or any component of wheat, such as, for example, wheat meal, wheat fiber, wheat hulls, wheat chaff, ground wheat, wheat germ, or any other portion of a wheat plant; canola, such as, for example, canola oil, canola meal, canola protein, canola hulls, ground canola, or any other portion of a canola plant; sunflower or a component of a sunflower plant; sorghum or a component of a sorghum plant; sugar beet or a component of a sugar beet plant; cane sugar or a component of a sugarcane plant; barley or a component of a barley plant; palm oil, palm kernel or a component of a palm plant; glycerol; corn steep liquor; a waste stream from an agricultural processing facility; lecithin; rumen protected fats; molasses; soy molasses; flax; peanuts; peas; oats; grasses, such as orchard grass and fescue; fish meal, meat & bone meal; feather meal; and poultry byproduct meal; and alfalfa and/or clover used for silage or hay, and various combinations of any of the feed ingredients set forth herein, or other feed ingredients generally known in the art. As it will be recognized in the art, a feed composition may further be supplemented with amino acids, vitamins, minerals, and other feed additives such as other types of enzymes, organic acids, essential oils, probiotics, prebiotics, antioxidants, pigments, anti-caking agents, and the like, as described further below.

A feed composition may be formulated for administration to any animal subject. Suitable subjects include all mammals, avian species, and aquaculture. Non-limiting examples of food animals include poultry (e.g., chickens, including broilers, layers, and breeders, ducks, game hens, geese, guinea fowl/hens, quail, and turkeys), beef cattle, dairy cattle, veal, pigs, goats, sheep, bison, and fishes. Suitable companion animals include, but are not limited to, cats, dogs, horses, rabbits, rodents (e.g., mice, rats, hamsters, gerbils, and guinea pigs), hedgehogs, and ferrets. Examples of research animals include rodents, cats, dogs, rabbits, pigs, and non-human primates. Non-limiting examples of suitable zoo animals include non-human primates, lions, tigers, bears, elephants, giraffes, and the like.

According to various embodiments of the present invention, the feed may be in any suitable form known in the animal feed art, and may be a wet or dry component. For example, according to certain embodiments, the feed composition may be in a form selected from the group consisting of a complete feed, a feed supplement, a feed additive, a premix, a top-dress, a tub, a mineral, a meal, a block, a pellet, a mash, a liquid supplement, a drench, a bolus, a treat, and combinations of any thereof. Additionally, a feed sample may optionally be ground before preparing a feed composition.

The dietary supplements or feed compositions may optionally comprise at least one additional nutritive and/or pharmaceutical agent. For instance, the at least one additional nutritive and/or pharmaceutical agent may be selected from the group consisting of vitamin, mineral, amino acid, antioxidant, probiotic, essential fatty acid, and pharmaceutically acceptable excipient. The compositions may include one additional nutritive and/or pharmaceutical component or a combination of any of the foregoing additional components in varying amounts. Suitable examples of each additional component are detailed below.

a. Vitamins

Optionally, the dietary supplement of the invention may include one or more vitamins. Suitable vitamins for use in the dietary supplement include vitamin C, vitamin A, vitamin E, vitamin B12, vitamin K, riboflavin, niacin, vitamin D, vitamin B6, folic acid, pyridoxine, thiamine, pantothenic acid, and biotin. The form of the vitamin may include salts of the vitamin, derivatives of the vitamin, compounds having the same or similar activity of a vitamin, and metabolites of a vitamin.

The dietary supplement may include one or more forms of an effective amount of any of the vitamins described herein or otherwise known in the art. Exemplary vitamins include vitamin K, vitamin D, vitamin C, and biotin. An “effective amount” of a vitamin typically quantifies an amount at least about 10% of the United States Recommended Daily Allowance (“RDA”) of that particular vitamin for a subject. It is contemplated, however, that amounts of certain vitamins exceeding the RDA may be beneficial for certain subjects. For example, the amount of a given vitamin may exceed the applicable RDA by 100%, 200%, 300%, 400%, 500% or more.

b. Minerals

In addition to the metal chelates or metal salts described in Section IA, the dietary supplement may include one or more minerals or mineral sources. Non-limiting examples of minerals include, without limitation, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In an exemplary embodiment, the mineral may be a form of calcium. Suitable forms of calcium include calcium alpha-ketoglutarate, calcium acetate, calcium alginate, calcium ascorbate, calcium aspartate, calcium caprylate, calcium carbonate, calcium chelates, calcium chloride, calcium citrate, calcium citrate malate, calcium formate, calcium glubionate, calcium glucoheptonate, calcium gluconate, calcium glutarate, calcium glycerophosphate, calcium lactate, calcium lysinate, calcium malate, calcium orotate, calcium oxalate, calcium oxide, calcium pantothenate, calcium phosphate, calcium pyrophosphate, calcium succinate, calcium sulfate, calcium undecylenate, coral calcium, dicalcium citrate, dicalcium malate, dihydroxycalcium malate, dicalcium phosphate, and tricalcium phosphate.

Generally speaking, the dietary supplement may include one or more forms of an effective amount of any of the minerals described herein or otherwise known in the art. An “effective amount” of a mineral typically quantifies an amount at least about 10% of the United States Recommended Daily Allowance (“RDA”) of that particular mineral for a subject. It is contemplated, however, that amounts of certain minerals exceeding the RDA may be beneficial for certain subjects. For example, the amount of a given mineral may exceed the applicable RDA by 100%, 200%, 300%, 400%, 500% or more. Typically, the amount of mineral included in the dietary supplement may range from about 1 mg to about 1500 mg, about 5 mg to about 500 mg, or from about 50 mg to about 500 mg per dosage.

c. Essential Fatty Acids

Optionally, the dietary supplement may include a source of an essential fatty acid. The essential fatty acid may be isolated or it may be an oil source or fat source that contains an essential fatty acid. In one embodiment, the essential fatty acid may be a polyunsaturated fatty acid (PUFA), which has at least two carbon-carbon double bonds generally in the cis-configuration. The PUFA may be a long chain fatty acid having at least 18 carbons atoms. The PUFA may be an omega-3 fatty acid in which the first double bond occurs in the third carbon-carbon bond from the methyl end of the carbon chain (i.e., opposite the carboxyl acid group). Examples of omega-3 fatty acids include alpha-linolenic acid (18:3, ALA), stearidonic acid (18:4), eicosatetraenoic acid (20:4), eicosapentaenoic acid (20:5; EPA), docosatetraenoic acid (22:4), n-3 docosapentaenoic acid (22:5; n-3DPA), and docosahexaenoic acid (22:6; DHA). The PUFA may also be an omega-5 fatty acid, in which the first double bond occurs in the fifth carbon-carbon bond from the methyl end. Exemplary omega-5 fatty acids include myristoleic acid (14:1), myristoleic acid esters, and cetyl myristoleate. The PUFA may also be an omega-6 fatty acid, in which the first double bond occurs in the sixth carbon-carbon bond from the methyl end. Examples of omega-6 fatty acids include linoleic acid (18:2), gamma-linolenic acid (18:3), eicosadienoic acid (20:2), dihomo-gamma-linolenic acid (20:3), arachidonic acid (20:4), docosadienoic acid (22:2), adrenic acid (22:4), and n-6 docosapentaenoic acid (22:5). The fatty acid may also be an omega-9 fatty acid, such as oleic acid (18:1), eicosenoic acid (20:1), mead acid (20:3), erucic acid (22:1), and nervonic acid (24:1).

In another embodiment, the essential fatty acid source may be a seafood-derived oil. The seafood may be a vertebrate fish or a marine organism, such that the oil may be fish oil or marine oil. The long chain (20C, 22C) omega-3 and omega-6 fatty acids are found in seafood. The ratio of omega-3 to omega-6 fatty acids in seafood ranges from about 8:1 to 20:1. Seafood from which oil rich in omega-3 fatty acids may be derived include, but are not limited to, abalone scallops, albacore tuna, anchovies, catfish, clams, cod, gem fish, herring, lake trout, mackerel, menhaden, orange roughy, salmon, sardines, sea mullet, sea perch, shark, shrimp, squid, trout, and tuna.

In yet another embodiment, the essential fatty acid source may be a plant-derived oil. Plant and vegetable oils are rich in omega-6 fatty acids. Some plant-derived oils, such as flaxseed oil, are especially rich in omega-3 fatty acids. Plant or vegetable oils are generally extracted from the seeds of a plant, but may also be extracted from other parts of the plant. Plant or vegetable oils that are commonly used for cooking or flavoring include, but are not limited to, acai oil, almond oil, amaranth oil, apricot seed oil, argan oil, avocado seed oil, babassu oil, ben oil, blackcurrant seed oil, Borneo tallow nut oil, borage seed oil, buffalo gourd oil, canola oil, carob pod oil, cashew oil, castor oil, coconut oil, coriander seed oil, corn oil, cottonseed oil, evening primrose oil, false flax oil, flax seed oil, grapeseed oil, hazelnut oil, hemp seed oil, kapok seed oil, lallemantia oil, linseed oil, macadamia oil, meadowfoam seed oil, mustard seed oil, okra seed oil, olive oil, palm oil, palm kernel oil, peanut oil, pecan oil, pequi oil, perilla seed oil, pine nut oil, pistachio oil, poppy seed oil, prune kernel oil, pumpkin seed oil, quinoa oil, ramtil oil, rice bran oil, safflower oil, sesame oil, soybean oil, sunflower oil, tea oil, thistle oil, walnut oil, or wheat germ oil. The plant derived oil may also be hydrogenated or partially hydrogenated.

In still a further embodiment, the essential fatty acid source may be an algae-derived oil. Commercially available algae-derived oils include those from Crypthecodinium cohnii and Schizochytrium sp. Other suitable species of algae, from which oil is extracted, include Aphanizomenon flos-aquae, Bacilliarophy sp., Botryococcus braunii, Chlorophyceae sp., Dunaliella tertiolecta, Euglena gracilis, Isochrysis galbana, Nannochloropsis salina, Nannochloris sp., Neochloris oleoabundans, Phaeodactylum tricornutum, Pleurochrysis carterae, Prymnesium parvum, Scenedesmus dimorphus, Spirulina sp., and Tetraselmis chui.

d. Amino Acids

The dietary supplement may optionally include from one to several amino acids. Suitable amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine or their hydroxy analogs. In certain embodiments, the amino acid will be selected from the essential amino acids. An essential amino acid is generally described as one that cannot be synthesized de novo by the organism, and therefore, must be provided in the diet. By way of non-limiting example, the essential amino acids for humans include: L-histidine, L-isoleucine, L-leucine, L-lysine, L-methionine, L-phenylalanine, L-valine and L-threonine.

e. Antioxidants

The dietary supplement may include one or more suitable antioxidants. As will be appreciated by a skilled artisan, the suitability of a given antioxidant will vary depending upon the species to which the dietary supplement will be administered. Non-limiting examples of antioxidants include ascorbic acid and its salts, ascorbyl palmitate, ascorbyl stearate, anoxomer, N-acetylcysteine, benzyl isothiocyanate, o-, m- or p-amino benzoic acid (o is anthranilic acid, p is PABA), butylated hydroxyanisole (BHA), butylated hydroxytoluene (BHT), caffeic acid, canthaxantin, alpha-carotene, beta-carotene, beta-caraotene, beta-apo-carotenoic acid, carnosol, carvacrol, catechins, cetyl gallate, chlorogenic acid, citric acid and its salts, p-coumaric acid, curcurin, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eugenol, ferulic acid, flavonoids, flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, propyl gallate, polyphosphates, quercetin, trans-resveratrol, rosmarinic acid, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanilic acid, 2,6-di-tert-butyl-4-hydroxymethylphenol (i.e., Ionox 100), 2,4-(tris-3′,5′-bi-tert-butyl-4′-hydroxybenzyl)-mesitylene (i.e., Ionox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivates, vitamin Q10, zeaxanthin, or combinations thereof.

Natural antioxidants that may be included in the dietary supplement include, but are not limited to, apple peel extract, blueberry extract, carrot juice powder, clove extract, coffeeberry, coffee bean extract, cranberry extract, eucalyptus extract, ginger powder, grape seed extract, green tea, olive leaf, parsley extract, peppermint, pimento extract, pomace, pomegranate extract, rice bran extract, rosehips, rosemary extract, sage extract, tart cherry extract, tomato extract, tumeric, and wheat germ oil.

f. Anti-Inflammatory Agents

The dietary supplement may optionally include at least one anti-inflammatory agent. In one embodiment, the anti-inflammatory agent may be a synthetic non-steroidal anti-inflammatory drug (NSAID) such as acetylsalicylic acid, dichlophenac, indomethacin, oxamethacin, ibuprofen, indoprofen, naproxen, ketoprofen, mefamanic acid, metamizole, piroxicam, and celecoxib. In an alternate embodiment, the anti-inflammatory agent may be a prohormone that modulates inflammatory processes. Suitable prohormones having this property include prohormone convertase 1, proopiomelanocortin, prohormone B-type natriuretic peptide, SMR1 prohormone, and the like. In another embodiment, the anti-inflammatory agent may be an enzyme having anti-inflammatory effects. Examples of anti-inflammatory enzymes include bromelain, papain, serrapeptidase, and proteolytic enzymes such as pancreatin (a mixture of tyrpsin, amylase and lipase).

In still another embodiment, the anti-inflammatory agent may be a peptide with anti-inflammatory effects. For example, the peptide may be an inhibitor of phospholipase A2, such as antiflammin-1, a peptide that corresponds to amino acid residues 246-254 of lipocortin; antiflammin-2, a peptide that corresponds to amino acid residues 39-47 of uteroglobin; S7 peptide, which inhibits the interaction between interleukin 6 and interleukin 6 receptor; RP1, a prenyl protein inhibitor; and similar peptides. Alternatively, the anti-inflammatory peptide may be cortistatin, a cyclic neuropeptide related to somatostatin, or peptides that correspond to an N-terminal fragment of SV-IV protein, a conserved region of E-, L-, and P-selectins, and the like. Other suitable anti-inflammatory preparations include collagen hydrolysates and milk micronutrient concentrates (e.g., MicroLactin® available from Stolle Milk Biologics, Inc., Cincinnati, Ohio), as well as milk protein hydrolysates, casein hydrolysates, whey protein hydrolysates, and plant protein hydrolysates.

In a further embodiment, the anti-inflammatory agent may be a probiotic that has been shown to modulate inflammation. Suitable immunomodulatory probiotics include lactic acid bacteria such as acidophilli, lactobacilli, and bifidophilli. In yet another embodiment, the anti-inflammatory agent may be a plant extract having anti-inflammatory properties. Non-limiting examples of suitable plant extracts with anti-inflammatory benefits include blueberries, boswella, black catechu and Chinese skullcap, celery seed, chamomile, cherries, devils claw, eucalyptus, evening primrose, ginger, hawthorne berries, horsetail, Kalopanax pictus bark, licorice root, tumeric, white wallow, willow bark, and yucca.

g. Probiotics

Probiotics and prebiotics may include yeast and bacteria that help establish an immune protective rumen or gut microflora as well as small oligosaccharides. By way of non-limiting example, yeast-derived probiotics and prebiotics include yeast cell wall derived components such as β-glucans, arabinoxylan isomaltose, agarooligosaccharides, lactosucrose, cyclodextrins, lactose, fructooligosaccharides, laminariheptaose, lactulose, β-galactooligosaccharides, mannanoligosaccharides, raffinose, stachyose, oligofructose, glucosyl sucrose, sucrose thermal oligosaccharide, isomalturose, caramel, inulin, and xylooligosaccharides. In an exemplary embodiment, the yeast-derived agent may be β-glucans and/or mannanoligosaccharides. Sources for yeast cell wall derived components include Saccharomyces bisporus, Saccharomyces boulardii, Saccharomyces cerevisiae, Saccharomyces capsularis, Saccharomyces delbrueckii, Saccharomyces fermentati, Saccharomyces lugwigii, Saccharomyces microellipsoides, Saccharomyces pastorianus, Saccharomyces rosei, Candida albicans, Candida cloaceae, Candida tropicalis, Candida utilis, Geotrichum candidum, Hansenula americana, Hansenula anomala, Hansenula wingei, and Aspergillus oryzae.

Probiotics and prebiotics may also include bacteria cell wall derived agents such as peptidoglycan and other components derived from gram-positive bacteria with a high content of peptidoglycan. Exemplary gram-positive bacteria include Lactobacillus acidophilus, Bifedobact thermophilum, Bifedobat longhum, Streptococcus faecium, Bacillus pumilus, Bacillus subtilis, Bacillus licheniformis, Lactobacillus acidophilus, Lactobacillus casei, Enterococcus faecium, Bifidobacterium bifidium, Propionibacterium acidipropionici, Propionibacteriium freudenreichii, and Bifidobacterium pseudolongum.

h. Herbals

Suitable herbals and herbal derivatives, as used herein, refer to herbal extracts, and substances derived from plants and plant parts, such as leaves, flowers and roots, without limitation. Non-limiting exemplary herbals and herbal derivatives include agrimony, alfalfa, aloe vera, amaranth, angelica, anise, barberry, basil, bayberry, bee pollen, birch, bistort, blackberry, black cohosh, black walnut, blessed thistle, blue cohosh, blue vervain, boneset, borage, buchu, buckthorn, bugleweed, burdock, capsicum, cayenne, caraway, cascara sagrada, catnip, celery, centaury, chamomile, chaparral, chickweed, chicory, chinchona, cloves, coltsfoot, comfrey, cornsilk, couch grass, cramp bark, culver's root, cyani, cornflower, damiana, dandelion, devils claw, dong quai, echinacea, elecampane, ephedra, eucalyptus, evening primrose, eyebright, false unicorn, fennel, fenugreek, figwort, flaxseed, garlic, gentian, ginger, ginseng, golden seal, gotu kola, gum weed, hawthorn, hops, horehound, horseradish, horsetail, hoshouwu, hydrangea, hyssop, iceland moss, irish moss, jojoba, juniper, kelp, lady's slipper, lemon grass, licorice, lobelia, mandrake, marigold, marjoram, marshmallow, mistletoe, mullein, mustard, myrrh, nettle, oatstraw, oregon grape, papaya, parsley, passion flower, peach, pennyroyal, peppermint, periwinkle, plantain, pleurisy root, pokeweed, prickly ash, psyllium, quassia, queen of the meadow, red clover, red raspberry, redmond clay, rhubarb, rose hips, rosemary, rue, safflower, saffron, sage, St. John's wort, sarsaparilla, sassafras, saw palmetto, skullcap, senega, senna, shepherd's purse, slippery elm, spearmint, spikenard, squawvine, stillingia, strawberry, taheebo, thyme, uva ursi, valerian, violet, watercress, white oak bark, white pine bark, wild cherry, wild lettuce, wild yam, willow, wintergreen, witch hazel, wood betony, wormwood, yarrow, yellow dock, yerba santa, yucca and combinations thereof.

i. Pigments

Suitable non-limiting pigments include actinioerythrin, alizarin, alloxanthin, β-apo-2′-carotenal, apo-2-lycopenal, apo-6′-lycopenal, astacein, astaxanthin, azafrinaldehyde, aacterioruberin, aixin, α-carotine, β-carotine, γ-carotine, β-carotenone, canthaxanthin, capsanthin, capsorubin, citranaxanthin, citroxanthin, crocetin, crocetinsemialdehyde, crocin, crustaxanthin, cryptocapsin, α-cryptoxanthin, β-cryptoxanthin, cryptomonaxanthin, cynthiaxanthin, decaprenoxanthin, dehydroadonirubin, diadinoxanthin, 1,4-diamino-2,3-dihydroanthraquinone, 1,4-dihydroxyanthraquinone, 2,2′-Diketospirilloxanthin, eschscholtzxanthin, eschscholtzxanthone, flexixanthin, foliachrome, fucoxanthin, gazaniaxanthin, hexahydrolycopene, hopkinsiaxanthin, hydroxyspheriodenone, isofucoxanthin, loroxanthin, lutein, luteoxanthin, lycopene, lycopersene, lycoxanthin, morindone, mutatoxanthin, neochrome, neoxanthin, nonaprenoxanthin, OH-Chlorobactene, okenone, oscillaxanthin, paracentrone, pectenolone, pectenoxanthin, peridinin, phleixanthophyll, phoeniconone, phoenicopterone, phoenicoxanthin, physalien, phytofluene, pyrrhoxanthininol, quinones, rhodopin, rhodopinal, rhodopinol, rhodovibrin, rhodoxanthin, rubixanthone, saproxanthin, semi-α-carotenone, semi-β-carotenone, sintaxanthin, siphonaxanthin, siphonein, spheroidene, tangeraxanthin, torularhodin, torularhodin methyl ester, torularhodinaldehyde, torulene, 1,2,4-trihydroxyanthraquinone, triphasiaxanthin, trollichrome, vaucheriaxanthin, violaxanthin, wamingone, xanthin, zeaxanthin, α-zeacarotene and combinations thereof.

j. Pharmaceutical Agents

Suitable non-limiting pharmaceutically acceptable agents include an acid/alkaline-labile drug, a pH dependent drug, or a drug that is a weak acid or a weak base. Examples of acid-labile drugs include statins (e.g., pravastatin, fluvastatin and atorvastatin), antiobiotics (e.g., penicillin G, ampicillin, streptomycin, erythromycin, clarithromycin and azithromycin), nucleoside analogs (e.g., dideoxyinosine (ddI or didanosine), dideoxyadenosine (ddA), dideoxycytosine (ddC)), salicylates (e.g., aspirin), digoxin, bupropion, pancreatin, midazolam, and methadone. Drugs that are only soluble at acid pH include nifedipine, emonapride, nicardipine, amosulalol, noscapine, propafenone, quinine, dipyridamole, josamycin, dilevalol, labetalol, enisoprost, and metronidazole. Drugs that are weak acids include phenobarbital, phenytoin, zidovudine (AZT), salicylates (e.g., aspirin), propionic acid compounds (e.g., ibuprofen), indole derivatives (e.g., indomethacin), fenamate compounds (e.g., meclofenamic acid), pyrrolealkanoic acid compounds (e.g., tolmetin), cephalosporins (e.g., cephalothin, cephalaxin, cefazolin, cephradine, cephapirin, cefamandole, and cefoxitin), 6-fluoroquinolones, and prostaglandins. Drugs that are weak bases include adrenergic agents (e.g., ephedrine, desoxyephedrine, phenylephrine, epinephrine, salbutamol, and terbutaline), cholinergic agents (e.g., physostigmine and neostigmine), antispasmodic agents (e.g., atropine, methantheline, and papaverine), curariform agents (e.g., chlorisondamine), tranquilizers and muscle relaxants (e.g., fluphenazine, thioridazine, trifluoperazine, chlorpromazine, and triflupromazine), antidepressants (e.g., amitriptyline and nortriptyline), antihistamines (e.g., diphenhydramine, chlorpheniramine, dimenhydrinate, tripelennamine, perphenazine, chlorprophenazine, and chlorprophenpyridamine), cardioactive agents (e.g., verapamil, diltiazem, gallapomil, cinnarizine, propranolol, metoprolol and nadolol), antimalarials (e.g., chloroquine), analgesics (e.g., propoxyphene and meperidine), antifungal agents (e.g., ketoconazole and itraconazole), antimicrobial agents (e.g., cefpodoxime, proxetil, and enoxacin), caffeine, theophylline, and morphine. In another embodiment, the drug may be a biphosphonate or another drug used to treat osteoporosis. Non-limiting examples of a biphosphonate include alendronate, ibandronate, risedronate, zoledronate, pamidronate, neridronate, olpadronate, etidronate, clodronate, and tiludronate. Other suitable drugs include estrogen, selective estrogen receptor modulators (SERMs), and parathyroid hormone (PTH) drugs. In yet another embodiment, the drug may be an antibacterial agent. Suitable antibiotics include aminoglycosides (e.g., amikacin, gentamicin, kanamycin, neomycin, netilmicin, streptomycin, and tobramycin), carbecephems (e.g., loracarbef), a carbapenem (e.g., certapenem, imipenem, and meropenem), cephalosporins (e.g., cefadroxil cefazolin, cephalexin, cefaclor, cefamandole, cephalexin, cefoxitin, cefprozil, cefuroxime, cefixi me, cefdinir, cefditoren, cefoperazone, cefotaxime, cefpodoxime, ceftazidime, ceftibuten, ceftizoxime, and ceftriaxone), macrolides (e.g., azithromycin, clarithromycin, dirithromycin, erythromycin, and troleandomycin), monobactam, penicillins (e.g., amoxicillin, ampicillin, carbenicillin, cloxacillin, dicloxacillin, nafcillin, oxacillin, penicillin G, penicillin V, piperacillin, and ticarcillin), polypeptides (e.g., bacitracin, colistin, and polymyxin B), quinolones (e.g., ciprofloxacin, enoxacin, gatifloxacin, levofloxacin, lomefloxacin, moxifloxacin, norfloxacin, ofloxacin, and trovafloxacin), sulfonamides (e.g., mafenide, sulfacetamide, sulfamethizole, sulfasalazine, sulfisoxazole, and trimethoprim-sulfamethoxazole), and tetracyclines (e.g., demeclocycline, doxycycline, minocycline, and oxytetracycline). In an alternate embodiment, the drug may be an antiviral protease inhibitor (e.g., amprenavir, fosamprenavir, indinavir, lopinavir/ritonavir, ritonavir, saquinavir, and nelfinavir). In still another embodiment, the drug may be a cardiovascular drug. Examples of suitable cardiovascular agents include cardiotonic agents (e.g., digitalis (digoxin), ubidecarenone, and dopamine), vasodilating agents (e.g., nitroglycerin, captopril, dihydralazine, diltiazem, and isosorbide dinitrate), antihypertensive agents (e.g., alpha-methyldopa, chlortalidone, reserpine, syrosingopine, rescinnamine, prazosin, phentolamine, felodipine, propanolol, pindolol, labetalol, clonidine, captopril, enalapril, and lisonopril), beta blockers (e.g., levobunolol, pindolol, timolol maleate, bisoprolol, carvedilol, and butoxamine), alpha blockers (e.g., doxazosin, prazosin, phenoxybenzamine, phentolamine, tamsulosin, alfuzosin, and terazosin), calcium channel blockers (e.g., amlodipine, felodipine, nicardipine, nifedipine, nimodipine, nisoldipine, nitrendipine, lacidipine, lercanidipine, verapamil, gallopamil, and diltiazem), and anticlot agents (e.g., dipyrimadole).

k. Excipients

A variety of commonly used excipients in dietary supplement formulations may be selected on the basis of compatibility with the active ingredients. Non-limiting examples of suitable excipients include an agent selected from the group consisting of non-effervescent disintegrants, a coloring agent, a flavor-modifying agent, an oral dispersing agent, a stabilizer, a preservative, a diluent, a compaction agent, a lubricant, a filler, a binder, taste masking agents, an effervescent disintegration agent, and combinations of any of these agents.

In one embodiment, the excipient is a binder. Suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof. The polypeptide may be any arrangement of amino acids ranging from about 100 to about 300,000 daltons.

In another embodiment, the excipient may be a filler. Suitable fillers include carbohydrates, inorganic compounds, and polyvinylpirrolydone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, and sorbitol.

The excipient may comprise a non-effervescent disintegrant. Suitable examples of non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth.

In another embodiment, the excipient may be an effervescent disintegrant. By way of non-limiting example, suitable effervescent disintegrants include sodium bicarbonate in combination with citric acid and sodium bicarbonate in combination with tartaric acid.

The excipient may comprise a preservative. Suitable examples of preservatives include antioxidants, such as a-tocopherol or ascorbate, and antimicrobials, such as parabens, chlorobutanol or phenol.

In another embodiment, the excipient may include a diluent. Diluents suitable for use include pharmaceutically acceptable saccharide such as sucrose, dextrose, lactose, microcrystalline cellulose, fructose, xylitol, and sorbitol; polyhydric alcohols; a starch; pre-manufactured direct compression diluents; and mixtures of any of the foregoing.

The excipient may include flavors. Flavors incorporated into the outer layer may be chosen from synthetic flavor oils and flavoring aromatics and/or natural oils, extracts from plants, leaves, flowers, fruits, and combinations thereof. By way of example, these may include cinnamon oils, oil of wintergreen, peppermint oils, clover oil, hay oil, anise oil, eucalyptus, vanilla, citrus oil, such as lemon oil, orange oil, grape and grapefruit oil, fruit essences including apple, peach, pear, strawberry, raspberry, cherry, plum, pineapple, and apricot.

In another embodiment, the excipient may include a sweetener. By way of non-limiting example, the sweetener may be selected from glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; sugar alcohols such as sorbitol, mannitol, sylitol, and the like.

In another embodiment, the excipient may be a lubricant. Suitable non-limiting examples of lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

The excipient may be a dispersion enhancer. Suitable dispersants may include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

Depending upon the embodiment, it may be desirable to provide a coloring agent in the outer layer. Suitable color additives include food, drug and cosmetic colors (FD&C), drug and cosmetic colors (D&C), or external drug and cosmetic colors (Ext. D&C). These colors or dyes, along with their corresponding lakes, and certain natural and derived colorants, may be suitable for use in the present invention depending on the embodiment.

The excipient may include a taste-masking agent. Taste-masking materials include, e.g., cellulose hydroxypropyl ethers (HPC) such as Klucel®, Nisswo HPC and PrimaFlo HP22; low-substituted hydroxypropyl ethers (L-HPC); cellulose hydroxypropyl methyl ethers (HPMC) such as Seppifilm-LC, Pharmacoat®, Metolose SR, Opadry YS, PrimaFlo, MP3295A, Benecel MP824, and Benecel MP843; methylcellulose polymers such as Methocel® and Metolose®; Ethylcelluloses (EC) and mixtures thereof such as E461, Ethocel®, Aqualon®-EC, Surelease; Polyvinyl alcohol (PVA) such as Opadry AMB; hydroxyethylcelluloses such as Natrosol®; carboxymethylcelluloses and salts of carboxymethylcelluloses (CMC) such as Aualon®-CMC; polyvinyl alcohol and polyethylene glycol co-polymers such as Kollicoat IR®; monoglycerides (Myverol), triglycerides (KLX), polyethylene glycols, modified food starch, acrylic polymers and mixtures of acrylic polymers with cellulose ethers such as Eudragit® EPO, Eudragit® RD100, and Eudragit® E100; cellulose acetate phthalate; sepifilms such as mixtures of HPMC and stearic acid, cyclodextrins, and mixtures of these materials. In other embodiments, additional taste-masking materials contemplated are those described in U.S. Pat. Nos. 4,851,226, 5,075,114, and 5,876,759, each of which is hereby incorporated by reference in its entirety.

In various embodiments, the excipient may include a pH modifier. In certain embodiments, the pH modifier may include sodium carbonate or sodium bicarbonate.

The dietary supplement or feed compositions detailed herein may be manufactured in one or several dosage forms. In an exemplary embodiment, the dosage form will be an oral dosage form. Suitable oral dosage forms may include a tablet, for example a suspension tablet, a chewable tablet, an effervescent tablet or caplet; a pill; a powder, such as a sterile packaged powder, a dispensable powder, and an effervescent powder; a capsule including both soft or hard gelatin capsules or non-animal derived polymers, such as hydroxypropyl methylcellulose capsules (i.e., HPMC) or pullulan; a lozenge; a sachet; a sprinkle; a reconstitutable powder or shake; a troche; pellets; granules; liquids; lick blocks; suspensions; emulsions; or semisolids and gels. Alternatively, the dietary supplement may be incorporated into a food product or powder for mixing with a liquid, or administered orally after only mixing with a non-foodstuff liquid. As will be appreciated by a skilled artisan, the dietary supplements, in addition to being suitable for administration in multiple dosage forms, may also be administered with various dosage regimens. Additionally, the antimicrobial clay may simply be added to any dosage form of a dietary supplement or feed composition.

The amount and types of ingredients (i.e., metal chelate, chondroprotective agents, vitamin, mineral, amino acid, antioxidant, yeast culture, and essential fatty acid), and other excipients useful in each of these dosage forms, are described throughout the specification and examples. It should be recognized that where a combination of ingredients and/or excipient, including specific amounts of these components, is described with one dosage form that the same combination could be used for any other suitable dosage form. Moreover, it should be understood that one of skill in the art would, with the teachings found within this application, be able to make any of the dosage forms listed above by combining the amounts and types of ingredients administered as a combination in a single dosage form or separate dosage forms and administered together as described in the different sections of the specification.

The dietary supplements of the present invention can be manufactured by conventional pharmacological techniques. Conventional pharmacological techniques include, e.g., one or a combination of methods: (1) dry mixing; (2) direct compression; (3) milling; (4) dry or non-aqueous granulation; (5) wet granulation; or (6) fusion. See, e.g., Lachman et al., The Theory and Practice of Industrial Pharmacy (1986). Other methods include, e.g., prilling, spray drying, pan coating, melt granulation, granulation, wurster coating, tangential coating, top spraying, extruding, coacervation and the like.

### II. Methods of Using

In another aspect, the present invention provides methods of using antimicrobial clays. An antimicrobial clay may be used alone, or may be formulated with various components to facilitate administration and effective use. An antimicrobial clay of the present disclosure may be formulated to facilitate administration and effective use. For instance, an antimicrobial clay, or compositions comprising an antimicrobial clay, may be powdered, pelleted, tableted, or hydrated to generate a paste to facilitate administration and use.

As described above, an antimicrobial clay may be used to control microbes as an alternative and complementary treatment to antibiotics. Non-limiting examples of uses for antimicrobial clays of the present disclosure include treating microbial infections in animals, controlling potentially harmful microbes in an animal's environment, improving growth performance of the animal, and controlling bacteria during fermentation.

In some embodiments, the present invention provides methods of using antimicrobial clay to control bacteria during fermentation for producing grain ethanol, alcoholic beverages, or other distilled beverages. Bacterial contamination is a major problem plaguing the efficient fermentation of sugar- or starch-containing feedstocks in the production of alcohol and alcoholic beverages. For some ethanol producers, bacterial contamination is the greatest obstacle to be overcome in their quest to become more profitable.

More than 500 different bacteria have been isolated and identified to be present at different stages of the fermentation process. Many bacteria enter the system with the various components used in fermentation. Bacterial contamination can reduce ethanol yields, necessitate expensive and time-consuming cleaning and decontamination of equipment, and cause spoilage of alcoholic beverages. For instance, lactic acid bacteria (LAB) such as Leuconostoc, Pediococcus, and Lactobacillus can also cause undesirable changes in wine flavor which renders the wine undrinkable. The growth of many species of LAB in alcoholic beverages can cause some serious spoilage.

Methods of using antimicrobial clay for controlling bacteria during fermentation comprise contacting the fermenting mixture with the antimicrobial clay. For instance, the antimicrobial clay may be added to the fermenting mixture as a powder, a pellet, or a tablet. Alternatively, the fermenting mixture may be passed through a filtering device comprising the antimicrobial clay to contact the fermenting mixture with the clay. The timing and duration of contacting a fermenting mixture with an antimicrobial clay can and will vary depending on the fermenting mixture and the fermentation process, and can be determined experimentally.

In other embodiments, the present invention provides methods of using antimicrobial clay to improve growth performance of the animal. In addition to controlling bacterial infections in animals, antibiotics are regularly administered to animals to increase efficiency and growth rate of the animals. In chicken feed, for example, tetracycline and penicillin show substantial improvement in egg production, feed efficiency and hatchability, but no significant effect on mortality.

Non-limiting examples of suitable animals include companion animals such as cats, dogs, rabbits, horses, and rodents such as gerbils; agricultural animals such as cows, dairy cows, dairy calves, beef cattle, pigs, goats, sheep, horses, deer; zoo animals such as primates, elephants, zebras, large cats, bears, and the like; research animals such as rabbits, sheep, pigs, dogs, primates, mice, rats and other rodents; avians, including but not limited to chickens, ducks, turkeys, ostrich, and emu; and aquatic animals chosen from fish and crustaceans including, but not limited to, salmon, shrimp, carp, tilapia, and shell fish. Preferred animals may be pigs, chickens, turkeys, dairy cattle, beef cattle, fish, and companion animals.

In yet other embodiments, the present invention provides methods of using antimicrobial clay in or on an animal to treat a microbial infection in the animal. Non-limiting examples of pathogenic bacteria that may be controlled using an antimicrobial clay of the present disclosure include Clostridium perfringens, Aeromonas hydrophila, Yersinia enterocolitica, Vibrio spp., Leptospira spp., Mycobacterium ulcerans, Listeria spp., pathogenic strains of E. coli, Pseudomonas spp. such as aeruginosa, Enterococcus spp., Salmonella spp., Campylobacter spp., Staphylococcus spp. such as epidermidis, S. aureus (MRSA), M. smegmatis, Streptococcus sp., Clostridia, and M. marinum. In a preferred alternative of the embodiments, an antimicrobial clay is administered to a pig to control enterotoxigenic E. coli in the pig. In another alternative of the embodiments, an antimicrobial clay is administered to a chicken to control necrotic enteritis in the chicken. In yet another alternative, an antimicrobial clay is administered to a pig to control influenza in the pig. In another alternative of the embodiments, an antimicrobial clay is administered to a pig to control scouring in the pig. Non-limiting examples of causes of scouring in pigs may include agalactia, Clostridia, Coccidiosis, Colibacillosis, Porcine epidemic diarrhea (PED) virus, porcine reproductive and respiratory syndrome virus (PRRSV), rotavirus, and transmittable gastro-enteritis (TGE) virus.

A method of using antimicrobial clay in an animal or in an animal's environment comprises contacting the animal's environment with the antimicrobial clay of the present disclosure or a composition comprising an antimicrobial clay of the present disclosure. Compositions comprising an antimicrobial agent may be as described in Section I above.

The timing and duration of administration of the composition of the invention to an animal or to an animal's environment can and will vary. For instance, a composition may be administered routinely throughout the period when the animal is raised to prevent a microbial infection. Alternatively, a composition may be administered after a microbial infection is detected and for the duration of the infection. A composition may also be administered at various intervals. For instance, a composition may be administered daily, weekly, monthly or over a number of months. In some embodiments, a composition is administered daily. In other embodiments, a composition is administered weekly. In yet other embodiments, a composition is administered monthly. In preferred embodiments, a composition is administered every three to six months. As it will be recognized in the art, the duration of treatment can and will vary depending on the progress of treatment.

In some embodiments, an antimicrobial clay composition may be administered to an environment associated with an animal for controlling pathogenic bacteria normally associated with such environments. For instance, an antimicrobial clay of the disclosure may be applied as a bedding amendment, an animal litter amendment, in a footbath normally used to prevent diseases in an animal's environment, as a poultice, dip, or aerosol to be applied on the animal, or applied to any other environment normally frequented by the animal. Pathogenic bacteria may be as described above.

Preferably, when an antimicrobial clay composition is administered to an animal, a method of the invention comprises oral administration of a feed supplement composition comprising clay to an animal. Alternatively, the antimicrobial clay composition may be orally administered to an animal via the animal's drinking water. One or more doses of a composition may be administered to an animal. As will be appreciated by one of skill in the art, a dose of a composition of the invention can and will vary depending on the body weight, sex, age and/or medical condition of the subject, the desired growth rate and efficiency desired, the microbial infection, the severity and extent of the microbial infection in the subject, the method of administration, and the duration of treatment, as well as the species of the subject.

Preferably, an antimicrobial clay composition is administered orally to an animal by adding the antimicrobial clay composition to a feed or supplement formulation and feeding the feed or supplement formulation to the animal. The amount of antimicrobial clay added to a feed or supplement composition may be as described in Section IB.

When administered orally with a feed or supplement formulation, an antimicrobial clay may be administered throughout the period of feeding the animal. Alternatively, an antimicrobial clay may be administered at specific periods during the growth and development of the animal. For instance, an antimicrobial clay may be administered during periods of heightened susceptibility of the animal to infection, such as during infancy.

When administered to an animal with a feed or supplement formulation, an antimicrobial clay composition may be administered at a rate of about 0.01 to about 100 grams per animal per day. For instance, an antimicrobial clay may be administered at a rate of about 1 to about 50 grams per animal per day, or about 1 to about 20 grams per animal per day. Preferably, an antimicrobial clay is administered at a rate of about 1 to about 15 grams per animal per day, more preferably from about 3 to about 10 grams per animal per day. When an antimicrobial clay composition comprises red clay, the clay may be administered at a rate of about 0.01 to about 50 grams per animal per day, or about 0.1 to about 20 grams per animal per day. Preferably, an antimicrobial clay composition comprising red clay is administered at a rate of about 0.1 to about 10 grams per animal per day, more preferably from about 0.3 to about 4 grams per animal per day.

An antimicrobial clay composition may also be administered to an animal at a rate of about 0.001 to about 100 grams/lb body weight/day. For instance, an antimicrobial clay may be administered at a rate of about 0.01 to about 50, or about 0.01 to about 10 grams/lb body weight/day. Preferably, an antimicrobial clay is administered at a rate of about 0.01 to about 10 grams/lb body weight/day, more preferably from about 0.05 to about 5 grams/lb body weight/day. When an antimicrobial clay composition comprises red clay, the clay may be administered at a rate of about 0.001 to about 10, or about 0.01 to about 5 grams/lb body weight/day. Preferably, an antimicrobial clay composition comprising red clay is administered at a rate of about 0.001 to about 1 grams/lb body weight/day, more preferably from about 0.025 to about 0.2 grams/lb body weight/day.

In some embodiments, the rate of administration of an antimicrobial clay of the disclosure may depend on the level of reducing agent in the antimicrobial clay. For instance, the level of reducing agent in the antimicrobial clay may be determined before administration to adjust the level of clay that may be used. For instance, the oxidation-reduction potential of an antimicrobial clay may be determined and the level of clay used in a method, composition, or formulation of the present disclosure is adjusted based on the oxidation-reduction potential of the clay. The oxidation-reduction potential of the clay may provide a general measure of the antimicrobial potential of a clay that may be used irrespective of the reducing agents present in the clay. Alternatively, the content of one or more specific reducing agents in the clay may be determined.

### DEFINITIONS

When introducing elements of the present disclosure, the articles “a,” “an,” “the,” and “said” are intended to mean that there are one or more of the elements. The use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms as used herein and in the claims shall include pluralities, and plural terms shall include the singular.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges can independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

As used herein, the terms “about” and “approximately” designate that a value is within a statistically meaningful range. Such a range can be typically within 20%, more typically still within 10%, and even more typically within 5% of a given value or range. The allowable variation encompassed by the terms “about” and “approximately” depends on the particular system under study and can be readily appreciated by one of ordinary skill in the art.

As used herein, “administering” is used in its broadest sense to mean contacting a subject with a composition disclosed herein.

As used herein, the term “antimicrobial activity” means microbicidal or microbiostatic activity or a combination thereof, against one or more microorganisms. Microbicidal activity refers to the ability to kill or cause irreversible damage to a target microorganism. Microbiostatic activity refers to the ability to inhibit the growth or proliferative ability of a target microorganism without necessarily killing or irreversibly damaging it.

The phrases “therapeutically effective amount” and “antimicrobial effective amount” are used interchangeably to mean an amount that is intended to qualify the amount of an agent or compound, that when administered, it will achieve the goal of healing an infection site, inhibiting the growth of a microorganism, or otherwise benefiting the recipient environment.

As used herein, the terms “treating,” “treatment,” or “to treat” each may mean to alleviate, suppress, repress, eliminate, prevent or slow the appearance of symptoms, clinical signs, or underlying pathology of a condition or disorder on a temporary or permanent basis. Preventing a condition or disorder involves administering an agent of the present invention to a subject prior to onset of the condition. Suppressing a condition or disorder involves administering an agent of the present invention to a subject after induction of the condition or disorder but before its clinical appearance. Repressing the condition or disorder involves administering an agent of the present invention to a subject after clinical appearance of the disease. Prophylactic treatment may reduce the risk of developing the condition and/or lessen its severity if the condition later develops. For instance, treatment of a microbial infection may reduce, ameliorate, or altogether eliminate the infection, or prevent it from worsening.

As used herein, the term “w/w” designates the phrase “by weight” and is used to describe the concentration of a particular substance in a mixture or solution.

As used herein, the term “subject” refers to a vertebrate species such as mammals, birds, reptiles, amphibians, and fish. The vertebrate species may be an embryo, a juvenile, or an adult. Examples of suitable mammals include, without limit, rodents, companion or domestic animals, livestock, and primates. Non-limiting examples of rodents include mice, rats, hamsters, gerbils, and guinea pigs. Non-limiting examples of livestock include goats, sheep, swine, cattle, llamas, and alpacas. Suitable primates include, but are not limited to, humans, capuchin monkeys, chimpanzees, lemurs, macaques, marmosets, tamarins, spider monkeys, squirrel monkeys, and vervet monkeys. Non-limiting examples of birds include chickens, turkeys, ducks, and geese.

As used herein, the terms “companion animal” or “domestic animal” refer to an animal typically kept as a pet for keeping in the vicinity of a home or domestic environment for company or protection, regardless of whether the animal is kept indoors or outdoors. Non-limiting examples of companion animals or domestic animals include, but are not limited to, dogs, cats, house rabbits, ferrets, and horses.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. “Purify” or “purification” in other embodiments means removing at least one contaminant from the composition to be purified. In this sense, purification does not require that the purified compound be homogenous, e.g., 100% pure.

Unless defined otherwise, all technical and scientific terms used herein have the same meaning as commonly understood by one of ordinary skill in the art to which the invention belongs. Although any methods, compositions, reagents, cells, similar or equivalent to those described herein can be used in the practice or testing of the invention, the preferred methods and materials are described herein.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Evaluation of Oral Administration of Antimicrobial Clay in Weanling Pigs

The antibacterial properties of an antimicrobial clay composition Product V (PV), in feed were evaluated in weanling pigs challenged with enterotoxigenic E. coli K88+ (ETEC). In essence, the antimicrobial clay composition was orally administered by adding PV to a basal diet at a rate of 0.2% by weight, and feeding to weanling pigs. The basal diet was as described in Table 1.

TABLE 1

| Ingredients | lbs/ton |
|---|---|
| Corn 600 14% moisture | 864.45 |
| Soybean 46% | 350.00 |
| Hamlet HP300 | 150.00 |
| betaGRO | 6.00 |
| Menhaden SS - Fish Meal | 96.00 |
| Lysine 98.5 | 5.60 |
| Methionine 99% DL | 3.05 |
| Threonine 98.5% | 2.20 |
| 21% Monocal | 13.50 |
| Limestone | 10.20 |
| Salt | 11.00 |
| Choline Chloride 60% | 1.00 |
| Nursery VTM 3# | 3.00 |
| Whey dried | 417.00 |
| Corn oil | 67.00 |
| Total | 2000.00 |

Analyzed Nutrients Composition

| | |
|---|---|
| ME, kcal/lb | 1529.85 |
| Crude Protein, % | 22.41 |
| TID Lysine, % | 1.38 |
| Avail Phos, % | 0.50 |
| Lactose | 15.01 |

The pigs were blocked into three treatment groups. The treatments included (1) pigs that were not challenged (NC) with ETEC but were also not treated with PV, (2) control (CON) pigs that were challenged with ETEC but were not treated with PV, and (3) pigs that were challenged and treated with PV (PROD). The pigs were blocked by body weight at weaning (15.5±3.0 lb.), and 9 pigs were used per treatment, with about 2-3 pigs/pen. All pigs challenged with ETEC were tested before the study to ascertain they were genetically susceptible to the bacteria.

On days 0-7, all pigs were fed their respective experimental diets to adapt the pigs to the diets. The pigs in the CON and PROD treatment groups were challenged by inoculating with 3 ml of ETEC (106 TCID50/ml) on day 7 and day 8. On day 11, the pigs were euthanized, and tissue was collected for analysis. The following parameters were assessed: pre-challenge and post-challenge average daily gain (ADG), pre-challenge and post-challenge average daily feed intake (ADFI), weaning and final body weight (BW), and mortality at 24, 48, and 72 hrs post-challenge.

Gut health of the animals was also assessed by measuring fecal consistency, gastrointestinal microbial activity, pH of gastrointestinal digesta in the ileum and colon, and immunohistological measurements in the ileum. Fecal consistency was evaluated using a four-point visual observation scale ranging from 0-3, with a score of 0 representing normal feces consistency, a score 1 representing soft feces, a score 2 representing mild diarrhea, and a score 3 representing severe diarrhea. The average fecal consistency score measured at 8, 24, 48, and 72 hrs after challenge. Gastrointestinal microbial activity was evaluated by measuring the number of total coliform bacteria in the ileal mucosa and colon, and the number ETEC count in the ileum. The visceral organ weights were also assessed to evaluate the effect of the treatment on the weight of the GI tract.

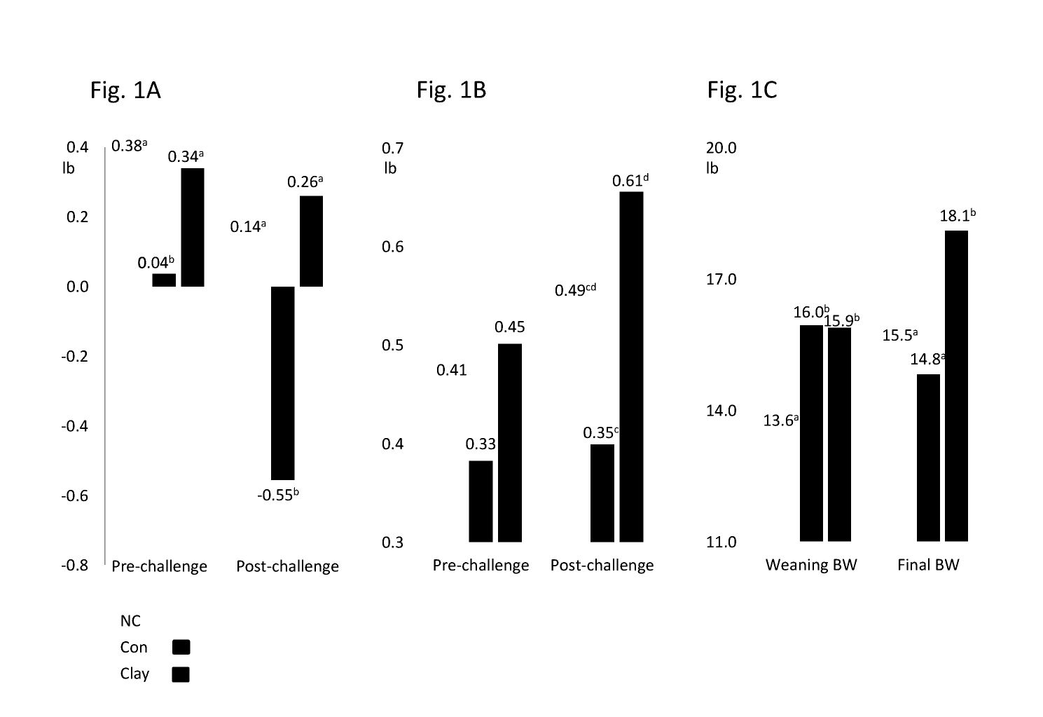

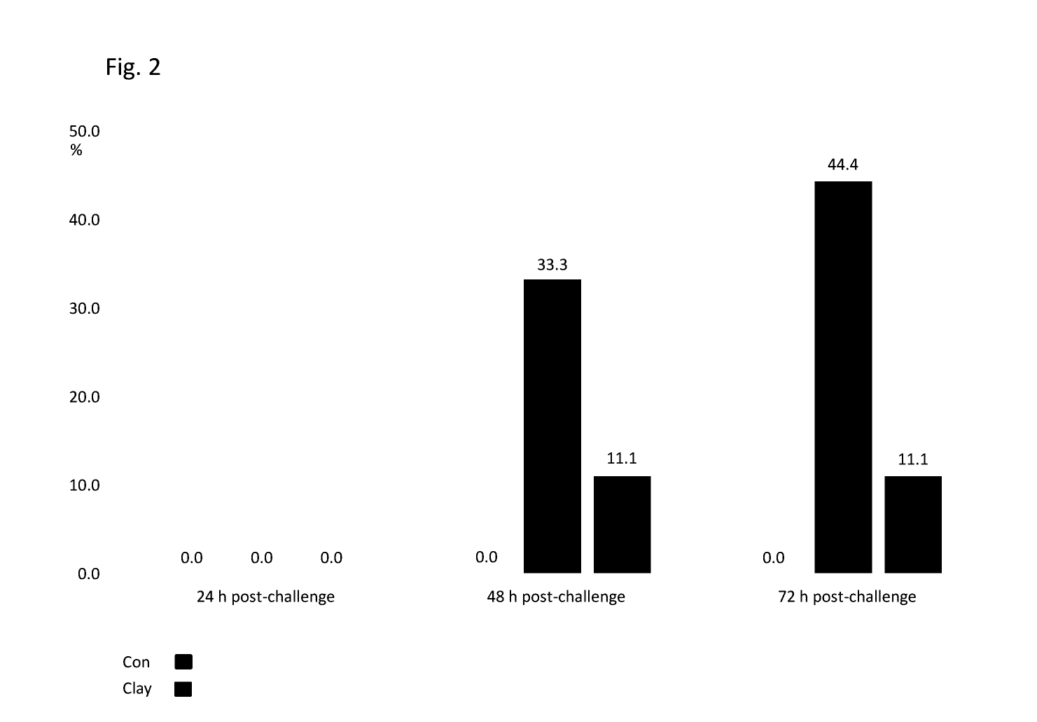

In all, the results show that feeding PV at 4.0 lb/ton to weanling pigs alleviates many of the negative effects from challenge of E. coli K88+. ADFI, post-challenge ADG, and final BW were improved in challenged pigs treated with PV when compared to challenged pigs that were not treated with PV (FIG. 1). In fact, ADFI and final BW were improved in challenged pigs treated with PV even when compared to pigs that were not challenged with ETEC but also not treated with PV. At 48 and 72 hrs post-challenge, mortality of challenged pigs treated with PV was significantly reduced when compared to challenged pigs that were not treated with PV (FIG. 2).

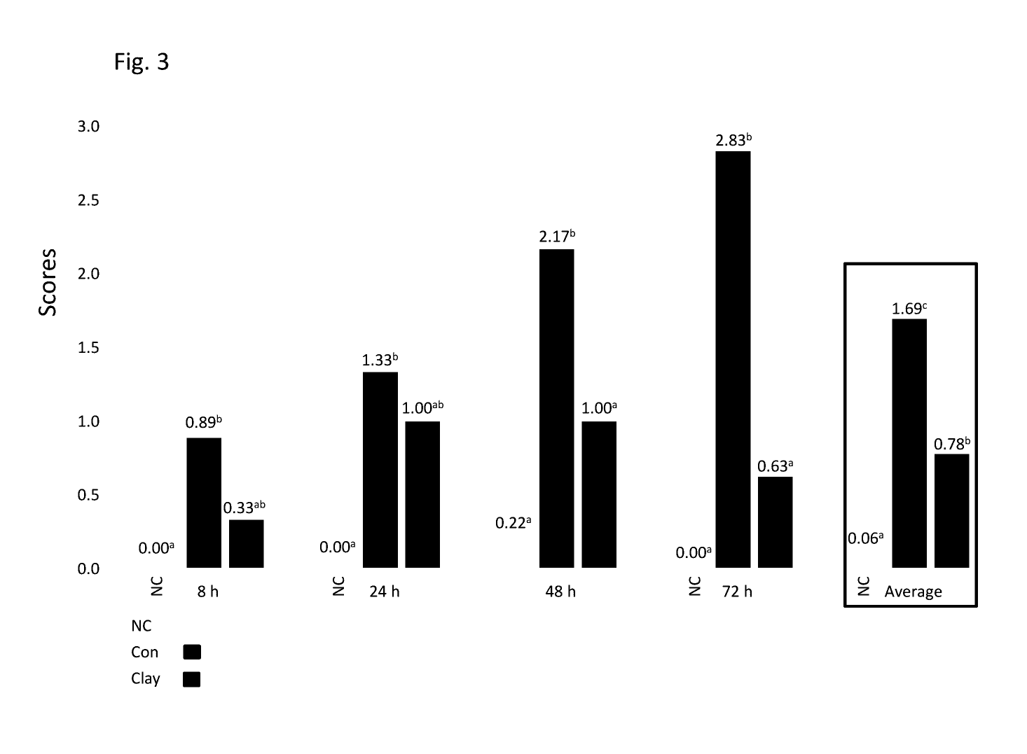

The results also show that gut health using all measures employed was also improved (FIG. 3). The fecal consistency score averaged over the four time-points was significantly improved in challenged animals administered PV when compared to challenged animals that were not treated with PV. Significantly, the fecal consistency score of challenged animals administered PV was never above 1. These results are especially striking when compared to the severe diarrhea observed in animals challenged with ETEC that were not treated with PV at 72 hrs post-challenge.

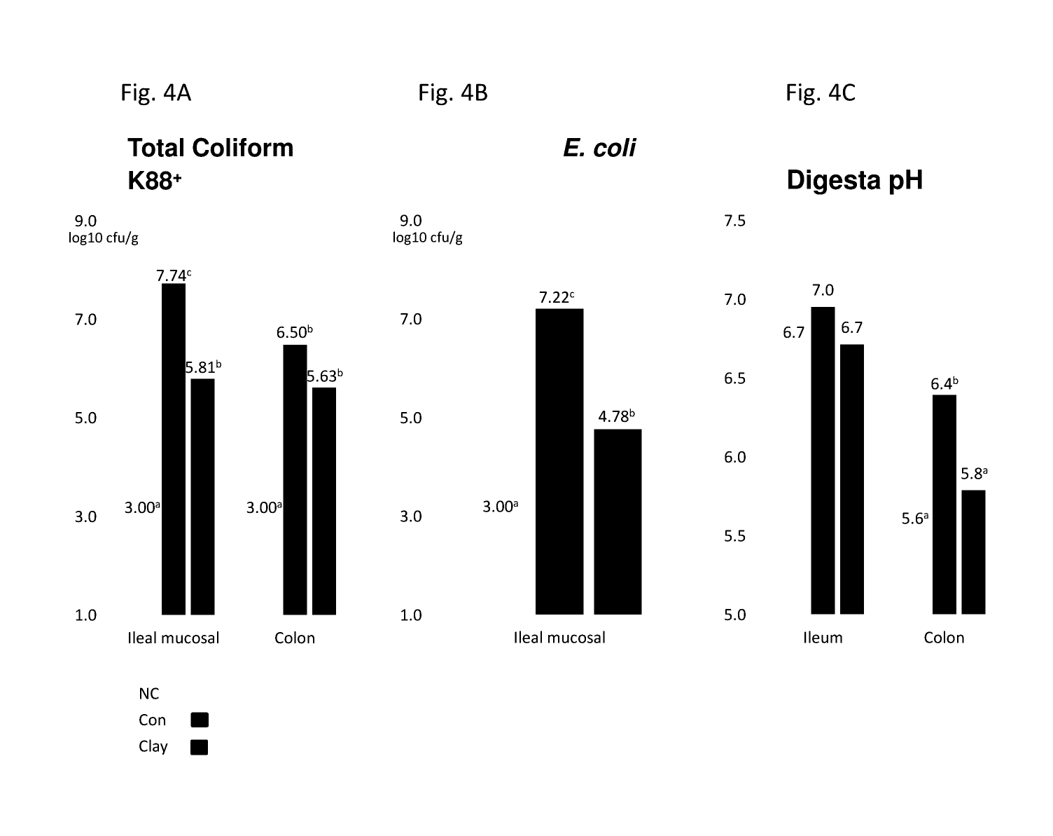

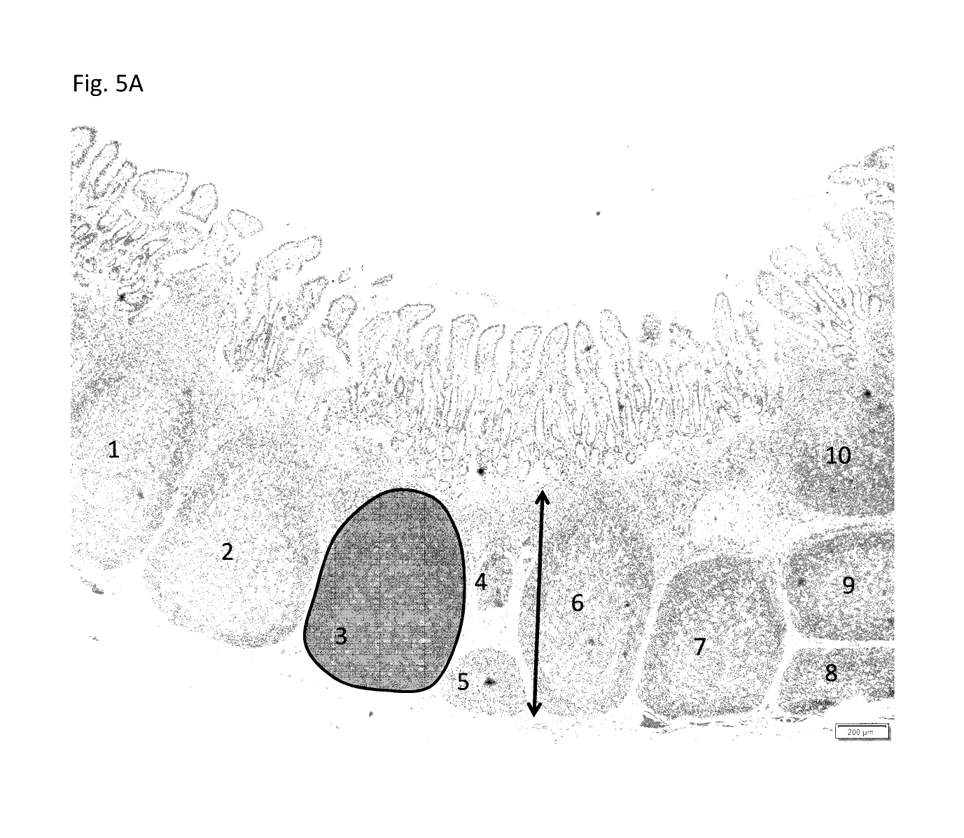

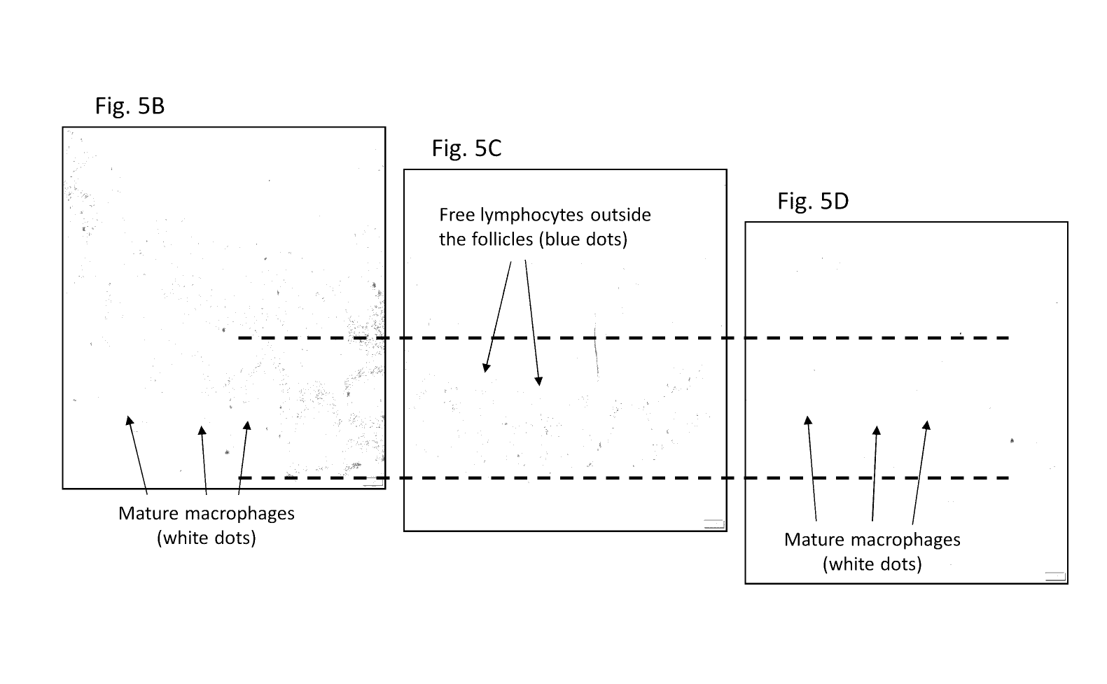

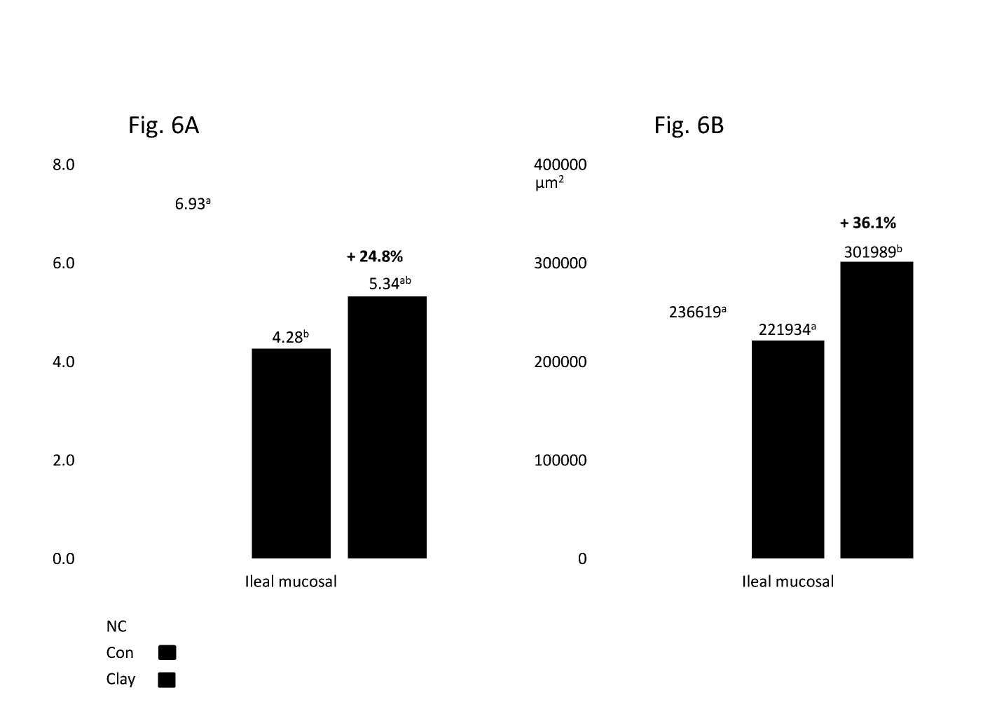

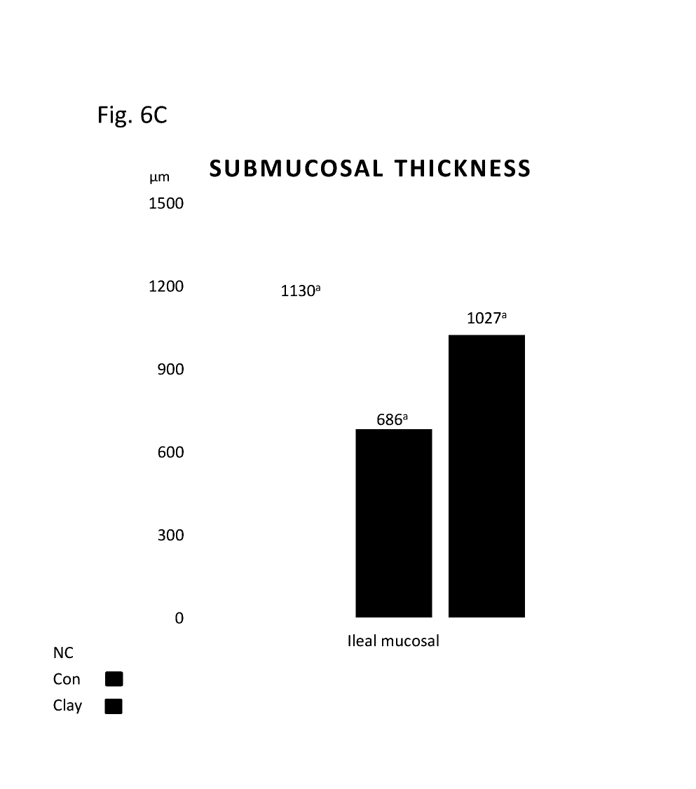

Total coliform count and E. coli K88+ count in the ileum was also significantly reduced in treated and challenged animals versus challenged animals that were not treated with PV (FIGS. 4A, B). Additionally, the treated animals maintained a healthy pH of gastrointestinal digesta in the ileum and colon, whereas the pH of colon digesta in challenged animals that were not treated with PV was significantly higher than both non-challenged and animals in the PROD treatment group (FIG. 4C). Treated animals also had larger and more follicles containing more macrophages in ileum (FIGS. 5, 6), signifying a better developed gut immune system. The treated pigs had about 24.8% more follicles than challenged pigs that were not treated. Additionally, the follicle area in treated pigs was about 36.1% larger than the area of follicles in challenged pigs that were not treated. In fact, the follicle area in treated pigs was even larger than the area of follicles in pigs that were not challenged with ETEC.

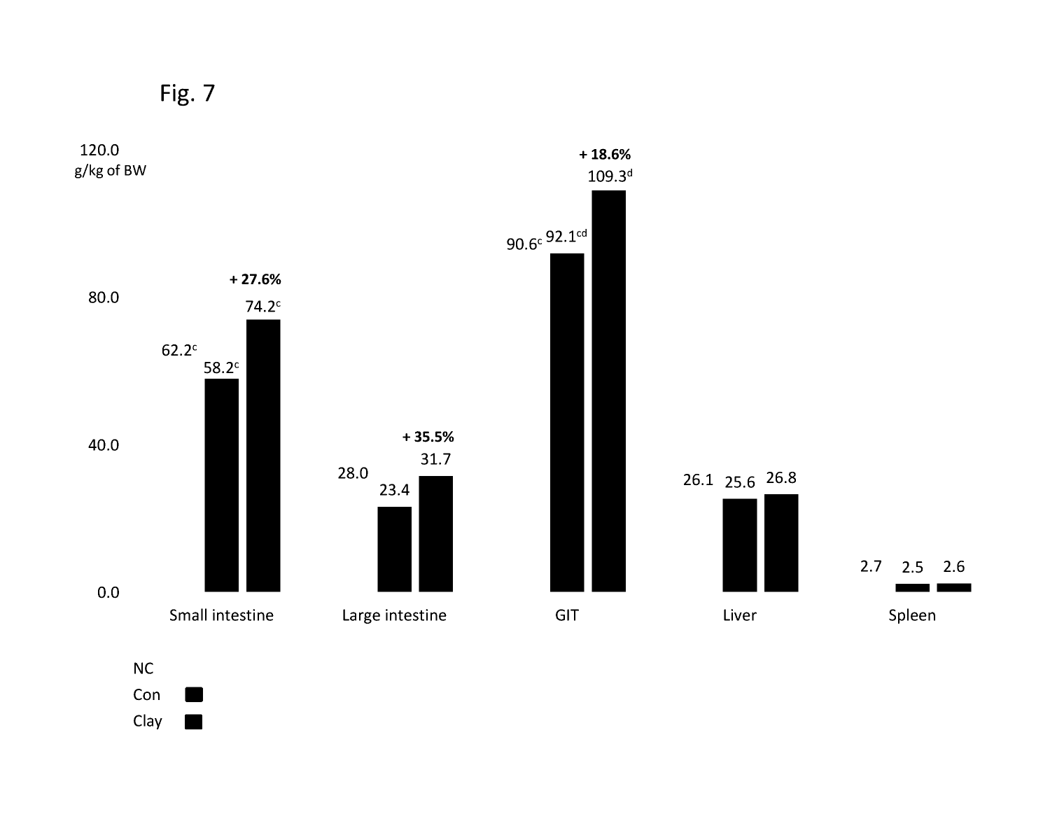

The weight of the total GI tract was also significantly improved in animals treated with PV and challenged with ETEC versus challenged animals that were not treated with PV (FIG. 7). In fact, the weight of the small intestine in animals treated with PV and challenged with ETEC was about 27.6% heavier than the weight of the small intestine in challenged pigs that were not treated. The weight of the large intestine in animals treated with PV and challenged with ETEC was about 35.5% heavier than the weight of the large intestine in challenged pigs that were not treated. The weight of the total GI tract in animals treated with PV and challenged with ETEC was about 18.6% heavier than the weight of the total GI tract in challenged pigs that were not treated. The weights of the spleen and liver were not affected.

### Example 2

### Evaluation of Oral Administration of Antimicrobial Clay for the Control of Necrotic Enteritis in Broiler Chickens

The effect of feeding PV for control of necrotic enteritis caused by Clostridium perfringens (Cp) was evaluated in broiler chickens. PV was orally administered by adding product to a basal diet at various rates, and feeding to broiler chickens. The diet was non-medicated corn/soybean meal, all-veg diet without organic acid, NSP enzyme, or DFM.

Day-of-hatch male Cobb 500 chicks were used for this study. All chicks were inoculated with coccidial challenge. Coccidia induce mucogenesis and promote the onset of necrotic enteritis by supporting Clostridium perfringens growth in chicks. The chicks were blocked into five treatment groups as shown in Table 2.

TABLE 2

| | | Coccidial | Clostridium |
|---|---|---|---|
| Treatment | Diet | Challenge | perfringens |
| 1 - Not Challenged | Basal | + | − |
| (NC) | | | |
| 2 - PV_0 | Basal | + | + |
| 3 - PV_1 | Clay at 1.0 lb/ton | + | + |
| 4 - PV_3 | Clay at 3.0 lb/ton | + | + |
| 5 - PV_6 | Clay at 6.0 lb/ton | + | + |

In all, 320 chicks were used in this study, with 8 birds/cage, and 8 cages/treatment, for a total of 64 birds per treatment. All the chicks were fed their respective experimental diets on days 0-14 to adapt the birds to the diets. On day 14, all the birds were orally inoculated with a coccidial inoculum containing approximately 5,000 oocysts of E. maxima per bird. On days 19, 20, and 21, birds in treatment groups 2-5 were orally inoculated with C. perfringens at 10⁸ cfu/ml once daily. On day 21, 3 birds from each cage were examined for the presence and degree of severity of necrotic enteritis lesions. Necrotic enteritis lesions were evaluated using a four-point scale ranging from 0-3, with a score of 0 representing normal, and a score of 3 representing the most severe lesions. Necrotic enteritis-related mortality was evaluated on day 28 when the trial was terminated. Body weight gain per cage was evaluated for the period between days 0 and 14, the period between days 14 and 21, and the period between days 21 and 28. Cumulative body weight gain per cage was also evaluated for the duration of the study (days 0 and 28). Feed conversion ratios were evaluated for the period between days 0 and 14 (pre-challenge), the period between days 14 and 28 (post-challenge), the period between days 14 and 21 (post-challenge), and the period between days 21 and 28 (post-challenge).

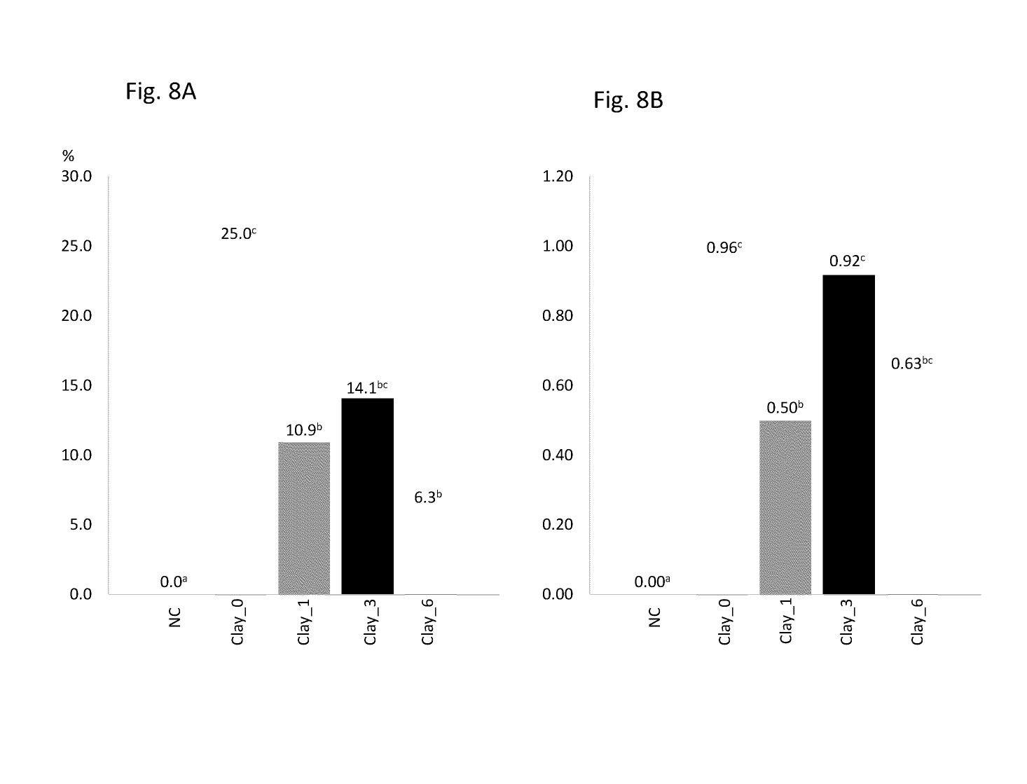

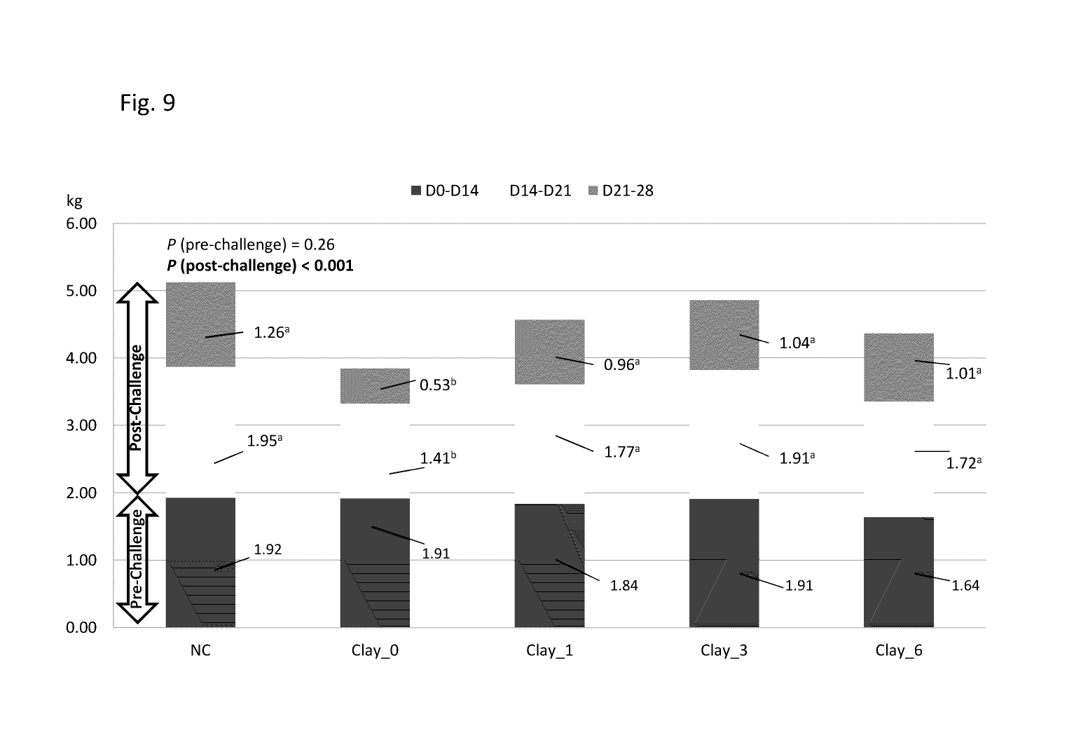

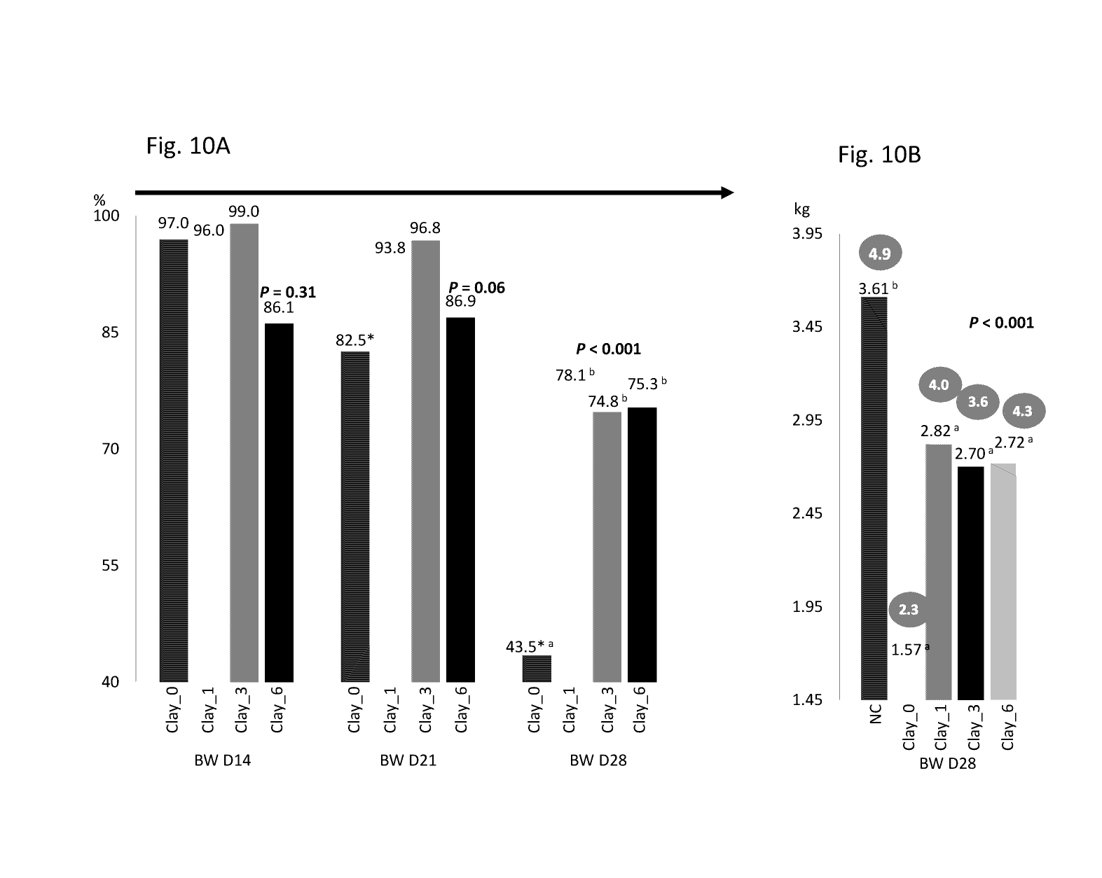

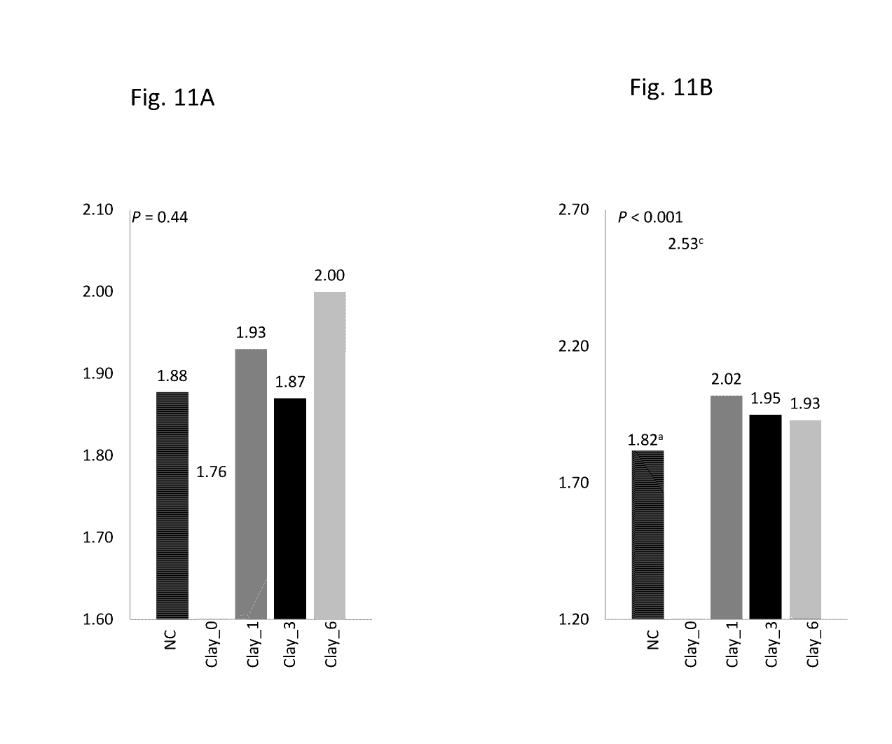

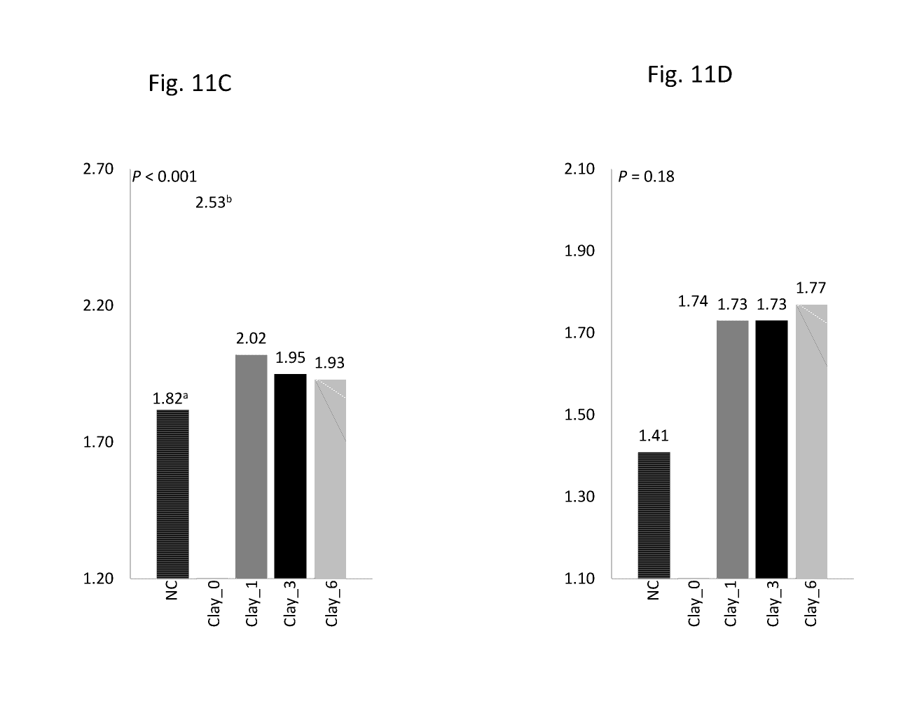

The results showed that treating birds with PV significantly reduced necrotic enteritis-related mortality when compared to birds that were not treated with PV (FIG. 8A). Also, reduction of necrotic enteritis-related mortality was lower for the treatment groups administered 1.0 and 6.0 lb/ton PV than the treatment group administered 3.0 lb/ton. The necrotic enteritis lesion score was also improved (FIG. 8B). The necrotic enteritis lesion score was lowest for the treatment group administered 1.0 lb/ton PV. Feeding chicks PV challenged with Cp also significantly improved body weight and cumulative body weight gain per cage when compared to challenged chicks that were not treated with PV (FIGS. 9, 10). In fact, the body weight and the cumulative body weight gain per cage of challenged birds treated with PV was not significantly different than the body weight or the cumulative body weight gain per cage of unchallenged birds. Additionally, although the body weight of challenged birds treated with PV was reduced at 28 days when compared to body weight of unchallenged birds, the weight difference was mainly due to the decrease in the number of birds in a cage, not lower weight/bird (FIG. 10B). Similarly, the feed conversion ratios for all post-challenge periods evaluated were improved for challenged birds fed PV when compared to challenged birds that were not treated with PV (FIG. 11).

### Example 3

### Evaluation of Oral Administration of Antimicrobial Clay on Growth Performance of Weanling Pigs

The effect of feeding PV with other nutritional supplements on growth performance of weanling pigs was evaluated. More specifically, growth performance of weanling pigs was evaluated when the pigs were administered PV with Evosure® Core, a specialty feed ingredient used for optimizing starter pig performance. In essence, PV was orally administered by adding PV at a rate of 2 lb/ton and Evosure® Core at a rate of 1 lb/ton to a basal diet, and feeding to weanling pigs. The basal diet was as described in Table 3. Total dietary ZnO level was 3000 ppm in basal diet.

TABLE 3

| | Basal Diet | | |
|---|---|---|---|
| | 9-15 lb | 15-25 lb | 25-40 lb |
| Ingredient, lb | Ration | Ration | Ration |
| Corn | 657.30 | 971.10 | 799.43 |
| Soybean meal, 46% | 387.00 | 539.00 | 462.01 |
| NHF Nursery Base | 850.00 | 300.00 | — |
| Bakery meal | — | — | 200.00 |
| Steamed rolled oats | — | 50.00 | — |
| Nursery VTM | 3.00 | 3.00 | 3.00 |
| DDGS | — | — | 400.00 |
| Fat | 68.40 | 63.60 | 46.59 |
| Mono Dical P | — | 20.00 | 20.71 |
| Limestone | — | 12.60 | 27.32 |
| Salt | — | 6.40 | 9.00 |
| Lysine HCL 78.8% | 8.40 | 8.50 | 13.02 |
| L-Threonine 98.5% | 4.40 | 3.80 | 4.21 |
| DL Methionine | 4.40 | 4.20 | 2.97 |
| L-Tryptophan | 1.10 | 0.70 | 1.17 |
| betaGRO | 6.00 | 2.50 | — |
| Zinc oxide, 72% | — | 5.00 | — |
| Tribasic copper | 0.80 | 0.76 | 0.81 |
| chloride | | | |
| Optiphos 2000 | — | — | 0.36 |
| Nursery | | | |
| Hemicell 2W | — | — | 0.50 |
| Mannanase | | | |
| Aureomycin 90 Meal | 8.90 | 8.90 | — |
| Pennchlor 90G | — | — | 8.90 |
| Total | 2000 | 2000 | 2000 |

The study was performed in three phases distributed over the experimental period as shown in Table 4.

TABLE 4

| | Phase 1; | Phase 2; | Phase 3; | |
|---|---|---|---|---|
| Initial BW | 9-15 ration | 15-25 ration | 25-40 ration | End BW |
| 11.9 lb | 7 days | 15 days | 11 days | 35.4 lb |

Mixed sex weanling pigs were used in this study and were blocked into four treatment groups as shown in Table 5. The pigs were blocked by body weight at weaning (11.9±0.5 lb.) and distributed into pens at 27 pigs/pen, 12 pens/treatment for a total of 1,296 animals.

TABLE 5

| Treatment | Evosure Core | Clay |
|---|---|---|
| 1: Control | — | — |
| 2: Evosure C | 1.0 lb/ton | — |
| 3: Clay | — | 2.0 lb/ton |
| 4: EvosureC/Clay | 1.0 lb/ton | 2.0 lb/ton |

The pigs were infected with flu during Phase 1, and total removal during this phase was as shown in Table 6.

TABLE 6

| Treatment | Total Removal, % |
|---|---|
| 1: Control | 3.1 |
| 2: Evosure Core | 5.2 |
| 3: Clay | 4.0 |
| 4: Evosure C/Clay | 4.9 |

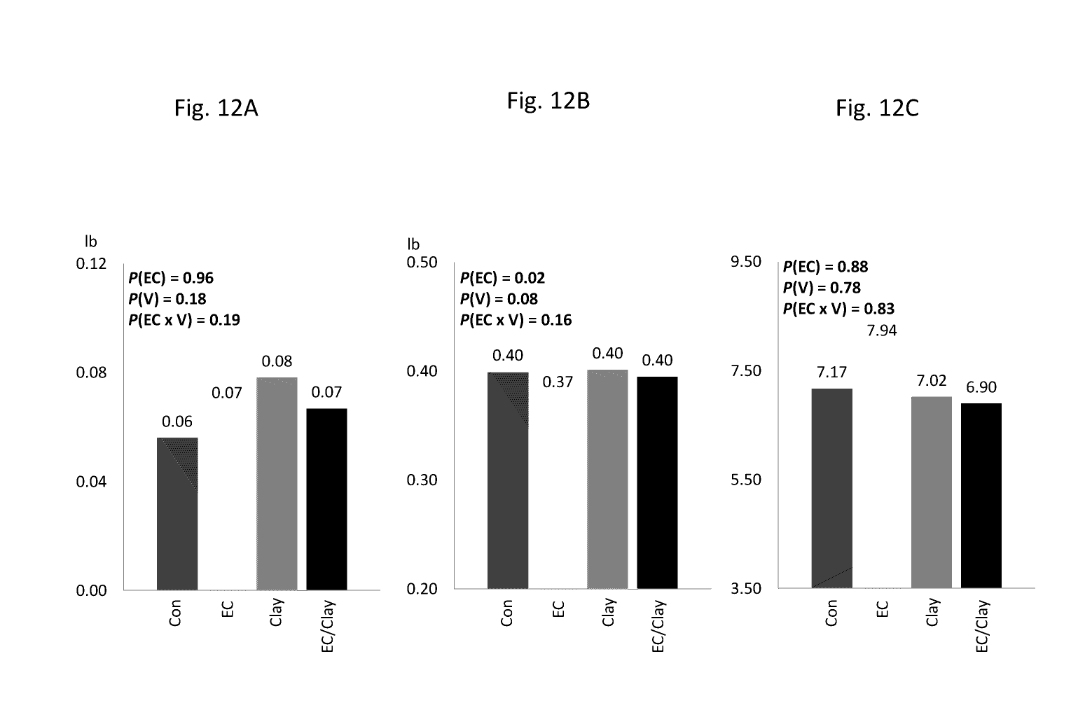

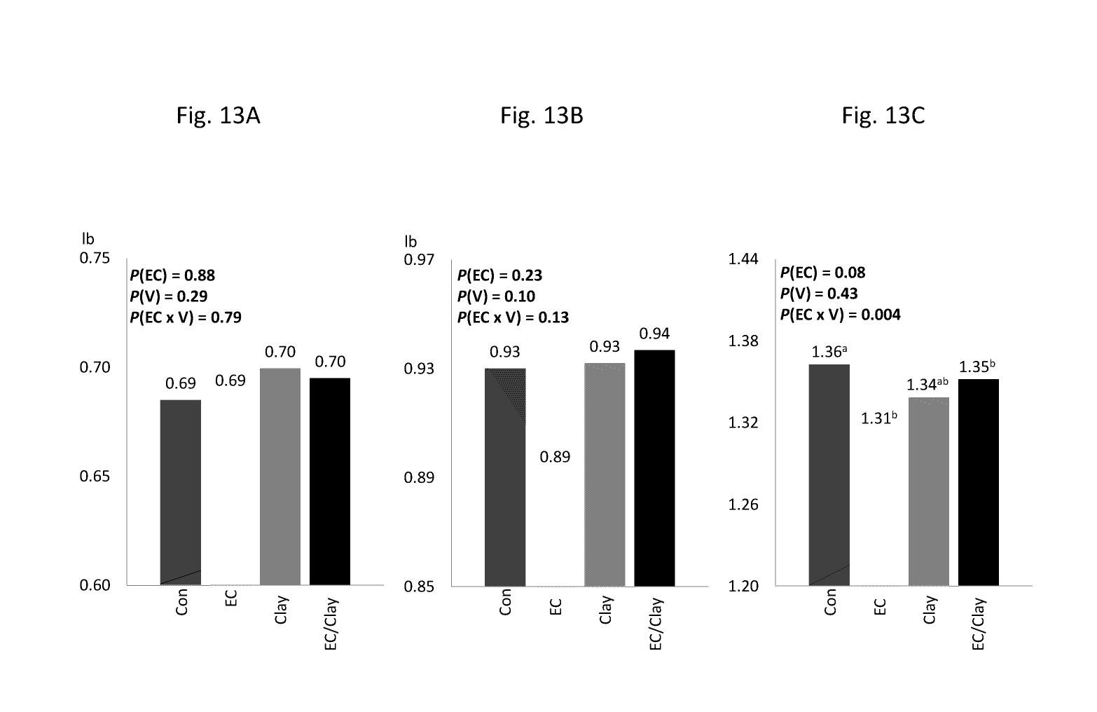

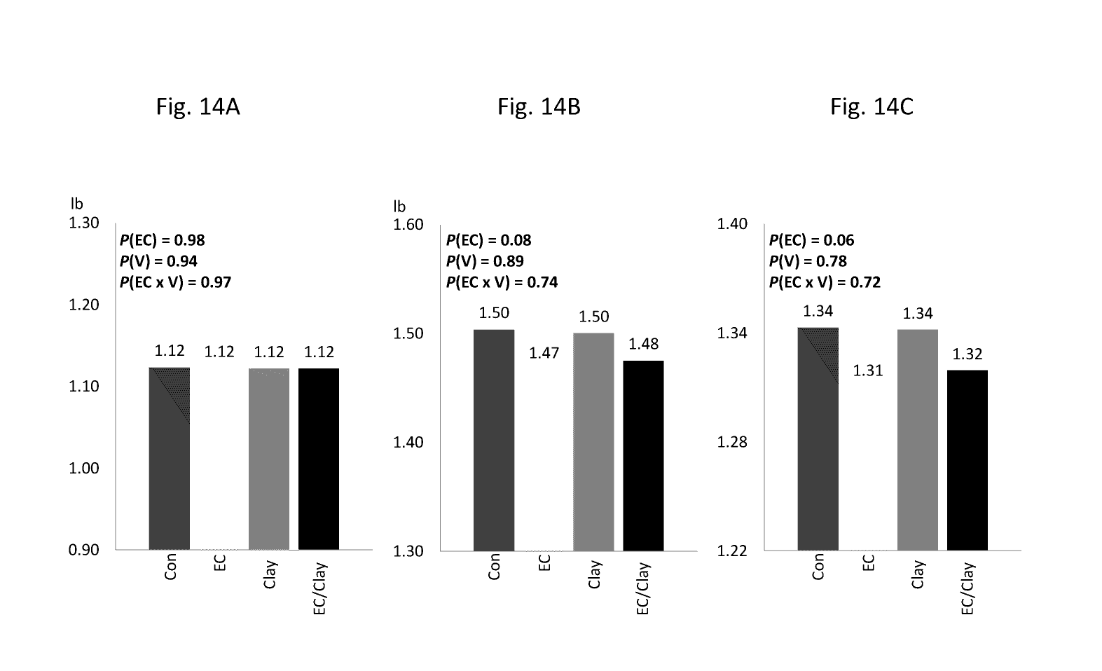

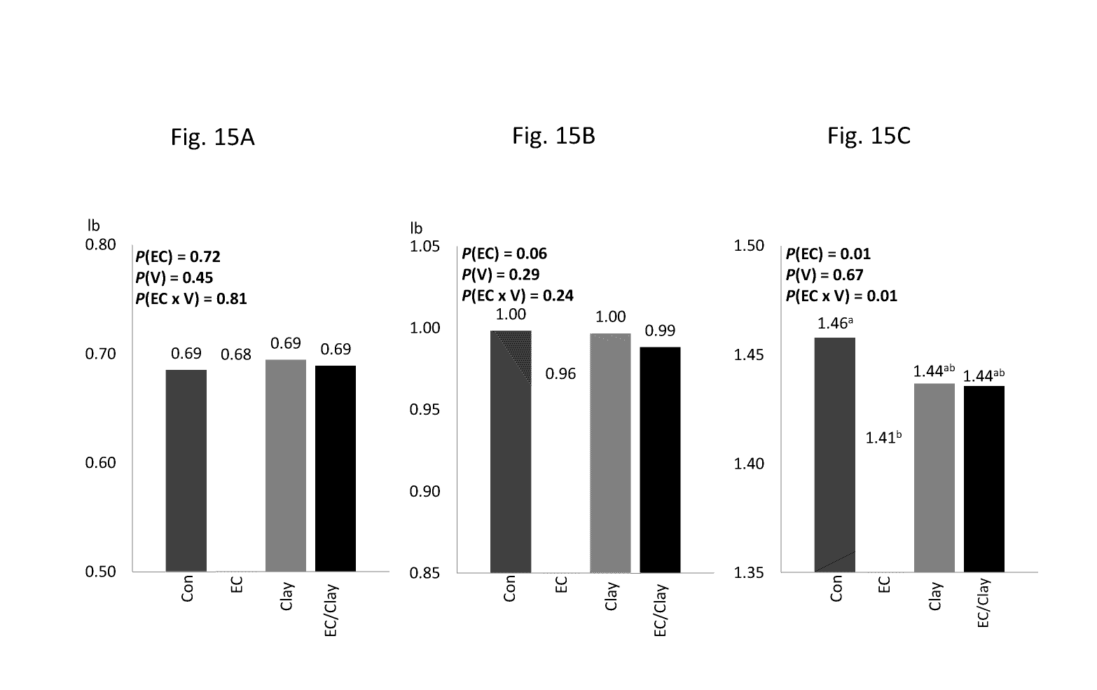

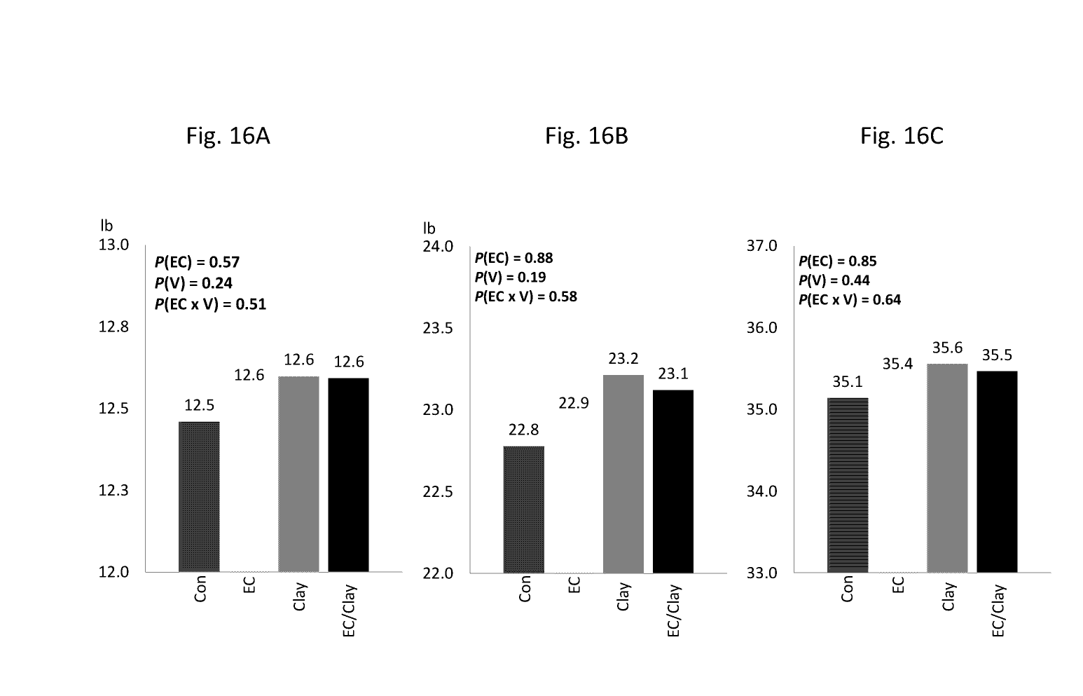

The average daily gain (ADG), the average daily feed intake (ADFI), and the feed to gain ratio (F:G) were assessed over the period of each phase and overall (FIGS. 12-16).

Overall, there was no significant effect on growth performance of weanling pigs from feeding Clay at 2.0 lb/ton when administered with Evosure® Core, regular medications, and high Zn (3000 ppm).

### Example 4

### Evaluation of Oral Administration of Antimicrobial Clay on Growth Performance of Weanling Pigs in the Presence of High and Low Levels of Zn

As described in Example 3, the levels of Zinc in the basal diet fed to pigs were high (3000 ppm). As a follow-up to the study of Example 3, a trial was conducted to evaluate the effects of feeding PV with high (3000 ppm) and low (1000 ppm) Zn levels on growth and performance of weanling pigs. Evosure® Core was not used in this study. PV was orally administered by adding PV at a rate of 2 lb/ton and to the basal diet described in Table 7.

TABLE 7

Basal Diet

| | | 15-25 lb |
|---|---|---|
| Item | 9-15 lb Ration | Ration |
| Ingredients, lb | | |
| Corn | 855.55 | 1030.30 |
| Soybean 46% | 350.00 | 450.00 |
| Hamlet HP300 | 150.00 | 100.00 |
| betaGRO | 6.00 | 3.00 |
| Fish Meal - Menhaden | 96.00 | 42.00 |
| Lysine 98.5 | 5.60 | 6.50 |
| DL Methionine 99% | 3.05 | 2.80 |
| Threonine 98.5% | 2.20 | 2.10 |
| 21% Monocal | 13.50 | 26.50 |
| Limestone | 10.20 | 15.80 |
| Salt | 11.00 | 7.10 |
| Choline Chloride 60% | 1.00 | 1.00 |
| Nursery VTM 3# | 3.00 | 3.00 |
| Whey dried | 417.00 | 250.00 |
| Corn oil | 67.00 | 51.00 |
| Aureomycin 90 Meal¹ | 8.90 | 8.90 |
| Total | 2000 | 2000 |

¹All diets contained 801 ppm of CTC from Aureomycin

The study was performed in two phases distributed over the experimental period as shown in Table 8.

TABLE 8

| | Phase 1; | Phase 2; | |
|---|---|---|---|
| Initial BW | 9-15 ration | 15-25 ration | End BW |
| 11.7 lb | 11 days | 15 days | 24.7 lb |

Mixed sex weanling pigs were used in this study and were blocked into four treatment groups as shown in Table 9. The pigs were blocked by body weight at weaning (11.7±1.5 lb.) and distributed into pens at 26-27 pigs/pen, 12 pens/treatment for a total of 1,296 animals.

TABLE 9

| Treatment | Zn | Clay |
|---|---|---|
| 1: CON/low Zn | 110 ppm¹ | — |
| 2: CON/High Zn | 3000 ppm² | — |
| 3: P_V/Low Zn | 110 ppm¹ | 2.0 lb/ton |
| 4: P_V/High Zn | 3000 ppm² | 2.0 lb/ton |

¹110 ppm of Zn was provided by adding 3 lb/ton nursery VTM containing 33,333 mg/lb of ZnO
²3000 ppm of Zn was provided by adding additional 8.03 lb/ton of ZnO (72%) to the diet

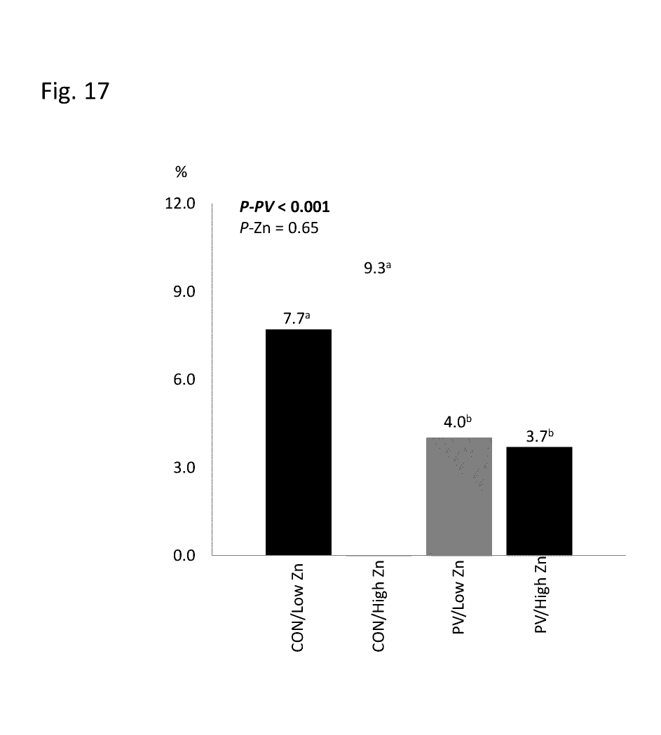

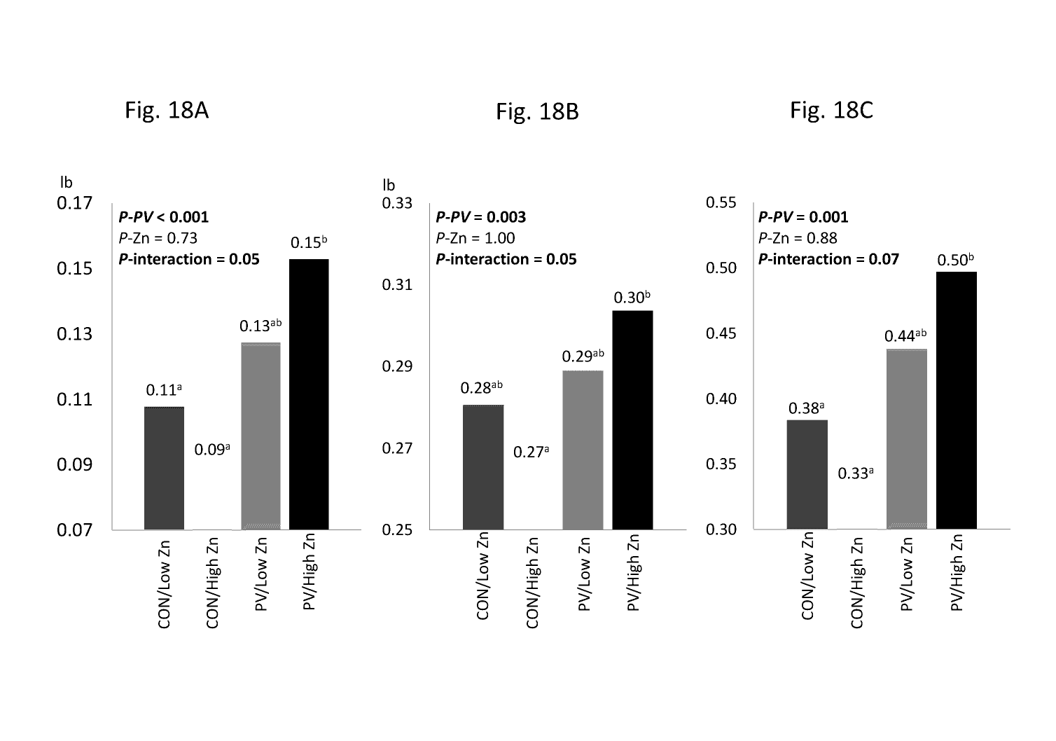

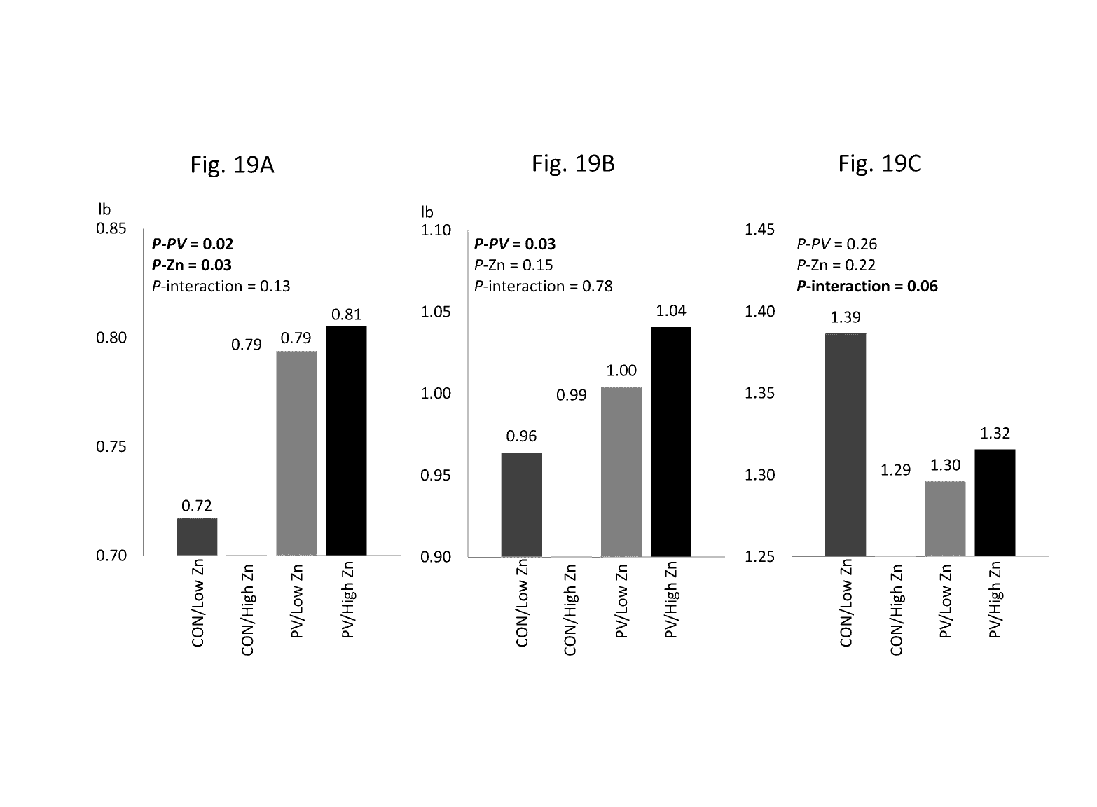

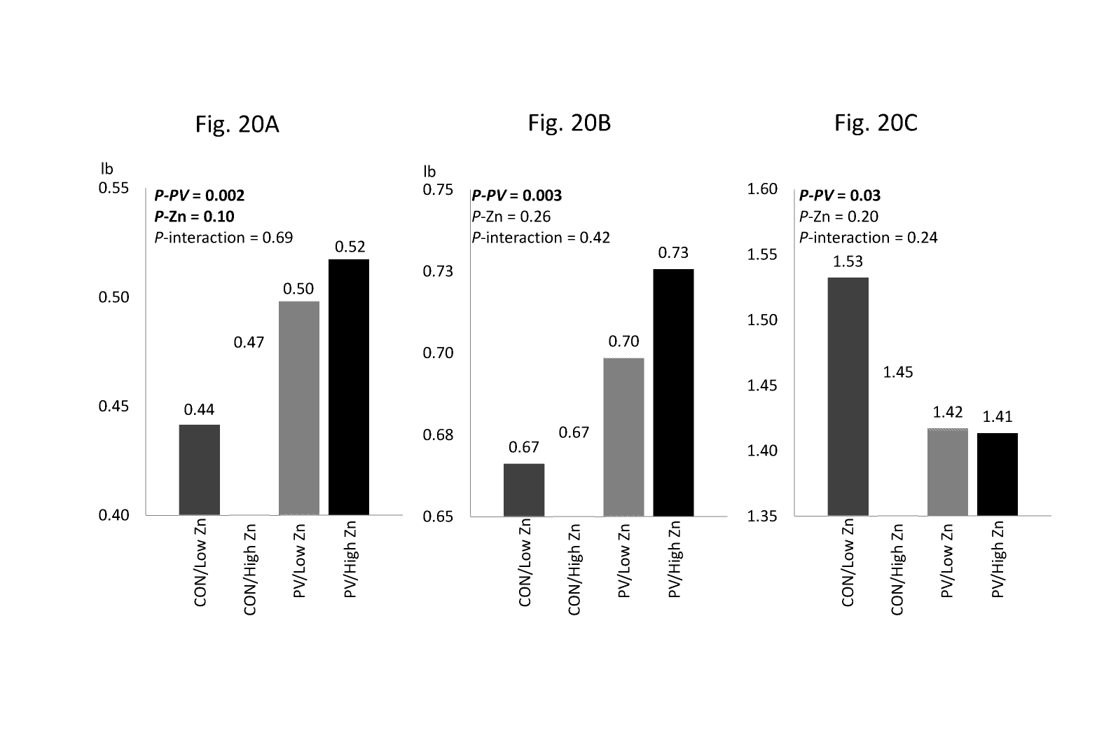

The pigs were experienced with scouring during Phase 1. Feeding PV significantly reduced removal rate (FIG. 17). The average daily gain (ADG), the average daily feed intake (ADFI), and the feed to gain ratio (F:G) were assessed over the period of each phase and overall (FIGS. 18-20). Table 10 numerically summarizes the data.

TABLE 10

| | | | | Main Effects of High Zn | | |
|---|---|---|---|---|---|---|
| | Main Effects of Clay | | | | | % Improvement |
| | | | % Improvement | Low | High | from |
| Responses | Control | Clay | from Clay | Zn | Zn | High Zn |

Phase 1; Day 0 to 11

| | | | | | | | | |
|---|---|---|---|---|---|---|---|---|
| ADG, lb | 0.10 | 0.14 | 41.9 | (P < 0.001) | 0.12 | 0.12 | 3.1 | (NS) |
| ADFI, lb | 0.27 | 0.30 | 8.4 | (P = 0.003) | 0.28 | 0.28 | 0.0 | (NS) |
| F:G | 0.36 | 0.47 | 30.3 | (P = 0.001) | 0.41 | 0.42 | 1.1 | (NS) |

Phase 2; Day 11 to 26

| | | | | | | | | |
|---|---|---|---|---|---|---|---|---|
| ADG, lb | 0.75 | 0.80 | 6.0 | (P = 0.02) | 0.76 | 0.80 | 5.6 | (P = 0.03) |
| ADFI, lb | 0.98 | 1.02 | 4.7 | (P = 0.03) | 0.98 | 1.01 | 3.1 | (NS) |
| F:G | 1.34 | 1.31 | −2.5 | (NS) | 1.34 | 1.30 | −2.8 | (NS) |

Overall; Day 0 to 26

| | | | | | | | | |
|---|---|---|---|---|---|---|---|---|
| ADG, lb | 0.46 | 0.51 | 11.1 | (P = 0.002) | 0.47 | 0.50 | 5.4 | (P = 0.10) |
| ADFI, lb | 0.67 | 0.71 | 6.5 | (P = 0.003) | 0.68 | 0.70 | 2.3 | (NS) |
| F:G | 1.49 | 1.42 | −5.0 | (P = 0.03) | 1.47 | 1.43 | −3.0 | (NS) |
| BW end of | 13.1 | 13.4 | 2.2 | (P = 0.02) | 13.2 | 13.3 | 0.6 | (NS) |
| Phase 1, lb | | | | | | | | |
| BW end of | 24.5 | 25.3 | 3.4 | (P = 0.04) | 24.5 | 25.3 | 3.3 | (P = 0.05) |
| Phase 2, lb | | | | | | | | |

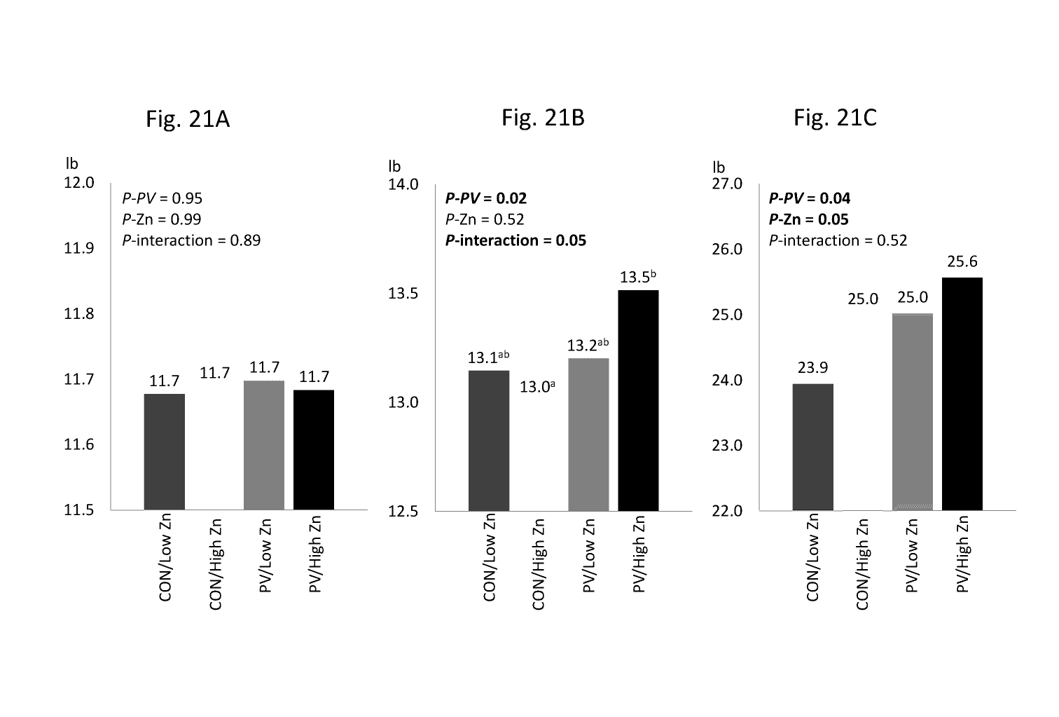

In summary, feeding PV at 2.0 lb/ton to weanling pigs significantly reduced removal rate by 4.6 percentage points and significantly improved overall growth performance. ADG was improved by 11.1%, ADFI was improved by 6.5%, feed efficiency was improved by 5.0%, and body weight was improved by 3.4%. Dietary supplementation of Zn at 3000 ppm did not affect removal rate, tended to improve overall ADG by 5.4%, and significantly improved BW by 3.3% (FIG. 21).

### Example 5

### Effect of Antimicrobial Clay on Rumen pH Using In Vitro Dry Matter Digestability Assay

Using the Sapienza Analytica, LLC (SALLC) ruminal analytics assay, the effect of antimicrobial clay test product (TP) on changes in pH and changes in dry matter disappearance (DMD) rates. In short, three dosage levels (25, 50, and 75 g/h/d) and 2 ruminal pH levels (5.5 and 6.0) were used. TP was not renewed, and the 2× daily feeding of total mixed ration (TMR) was not simulated over the time course of the experiment. Starting pH of composite rumen fluid was adjusted to approximately 5.5 or 6.0 using acetic:propionic acid mixture (10:1 molar ratio). The eight treatments used were as follows:
Incubation with equivalent of TP at 25 g/h/d at pH 6.0;
Incubation with equivalent of TP at 25 g/h/d at pH 5.5;
Incubation with equivalent of TP at 50 g/h/d at pH 6;
Incubation with equivalent of TP at 50 g/h/d at pH 5.5;
Incubation with equivalent of TP at 75 g/h/d at pH 6;
Incubation with equivalent of TP at 75 g/h/d at pH 5.5
BLANK which will be all reagents and dilutions at pH 6;
BLANK which will be all reagents and dilutions at pH 5.5.

The 25, 50, and 75 g/h/d doses correspond to weights of TP shown in Table 11.

TABLE 11

| Dosage of TP | | |
|---|---|---|
| (g/h/d) | % IV TMR | g/kg TMR |
| 25 | 0.167 | 1.670 |
| 50 | 0.333 | 3.330 |
| 75 | 0.500 | 5.000 |
| Blank | 0 | 0 |

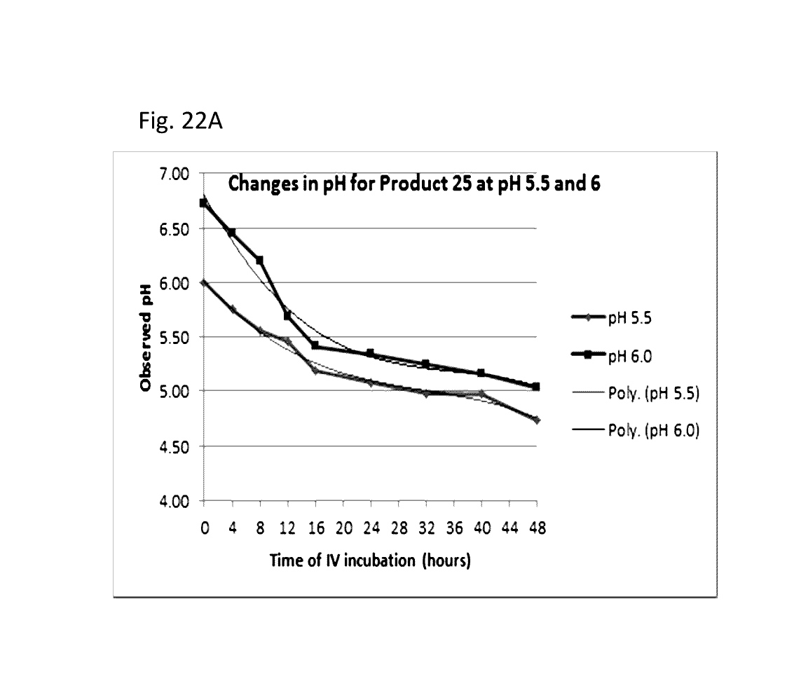

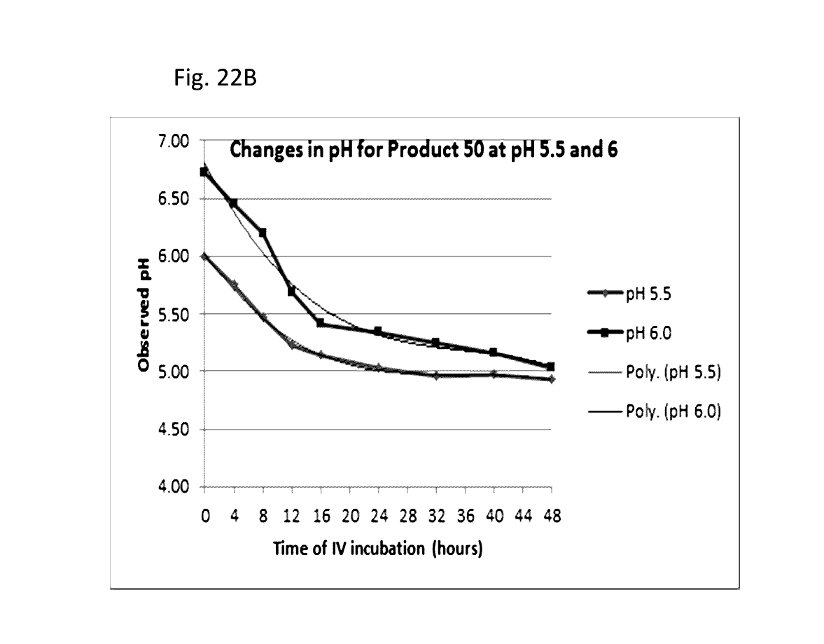

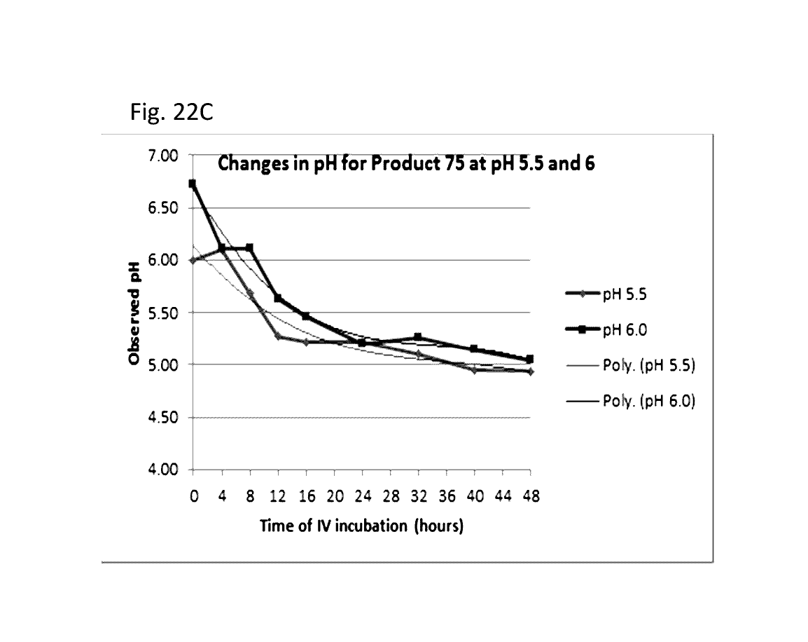

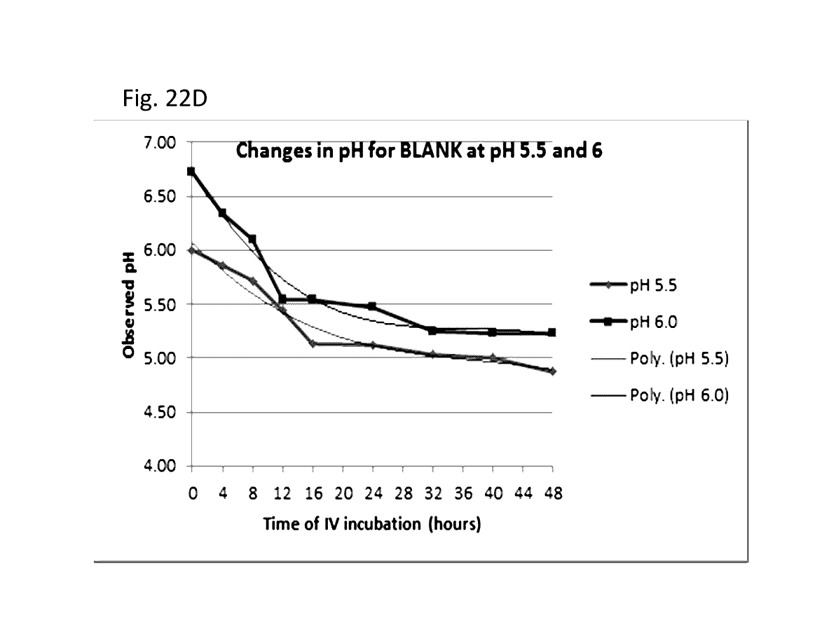

To monitor pH, mixtures of TMR were prepared in accordance with test product formulations. IV solutions were prepared and initial pH values were recorded, and composite rumen fluid sample was added to IV solutions. IV bags containing TMR-by-TP were added to IV solution plus rumen fluid, and measurements of pH were recorded each 4 hours over 48 hours for each replicate. As can be seen in FIG. 22, some trends in difference between the groups were observed, although they were not statistically significant. The p-values at pH 5.5 for the TP25, TP50, TP75 and Blank groups are shown in Table 12, and the p-values at pH 6.0 for the TP25, TP50, TP75 and Blank groups are shown in Table 13. At both pH 5.5 (Table 12) and pH 6.0 (Table 13), the TP75 overall time course appeared to be numerically different from BLANK.

Table 14 shows the p-values at pH 6.0 at asymptote. The asymptote for TP75, which attained stable pH at 32 hours, appeared to be different from TP25, TP50 and Blank at pH 6.0.

Based on the above experiment (FIG. 22), there may be some influences upon acid production caused by changes in microbial fermentation by TP75 because the TP75 treatment group reached a stable pH, and the stable pH is slightly higher compared to the other treatment groups. This may be because TP75 may be altering the rumen microbial balance.

### Example 6

### Evaluation of the Effect of the Test Product (TP) on Dry Matter Disappearance (DMD) During In Vitro Incubation

Measuring DMD is a proxy for measuring microbial activity. The effect of TP on DMD was measured. Specifically, the effect of different dosage levels of TP on DMD was measured in vitro (IV) over a 48 hour period. A lower DMD may be an indicator of decreased microbial activity by TP. Mixtures of TMR (total mixed ration) were prepared in accordance with test product formulations for 8 treatment groups, as described in Example 5. The IV solutions were prepared and initial pH recorded. Composite rumen fluid samples were added to the IV solution, which was added to the IV bags containing TMR-by-IP for each treatment group. The DMD was measured at 0, 4, 12, 16, 24, 32, 40 and 48 hours (every 4 hours) for each replicate in the 8 treatment groups.

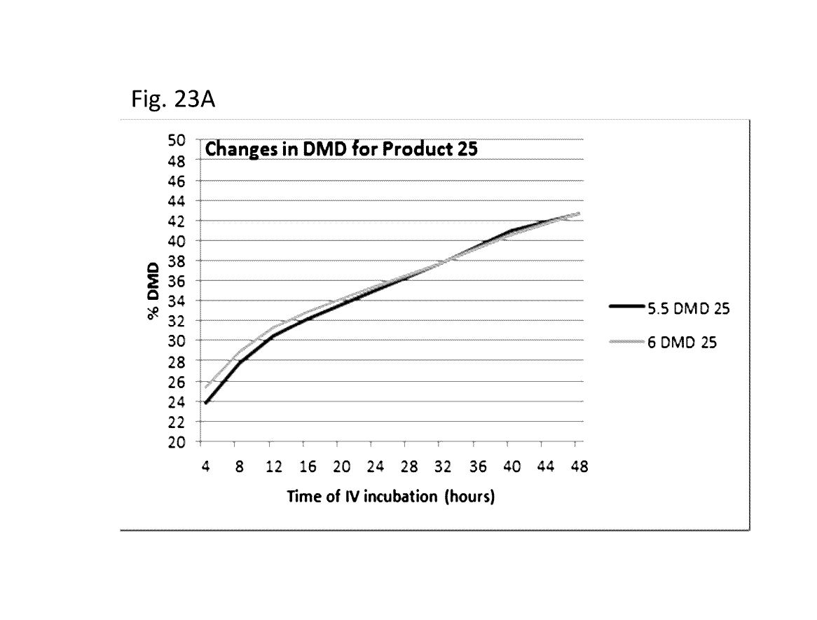

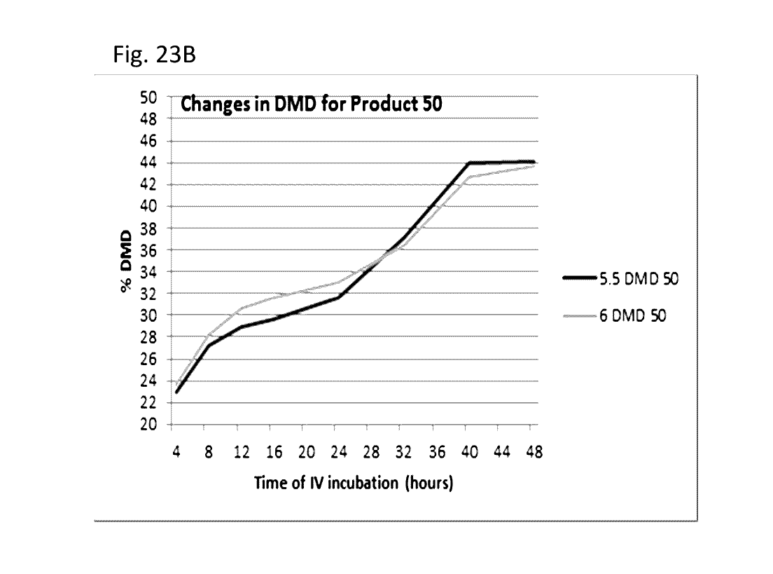

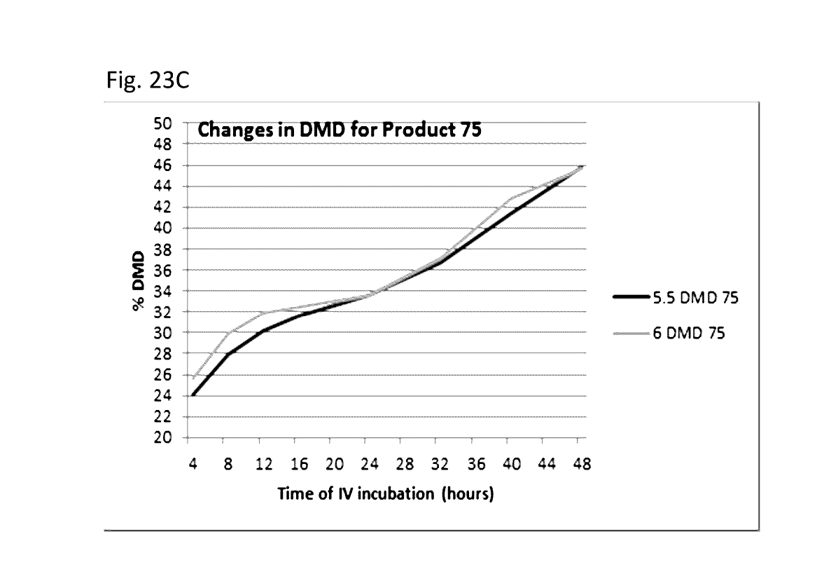

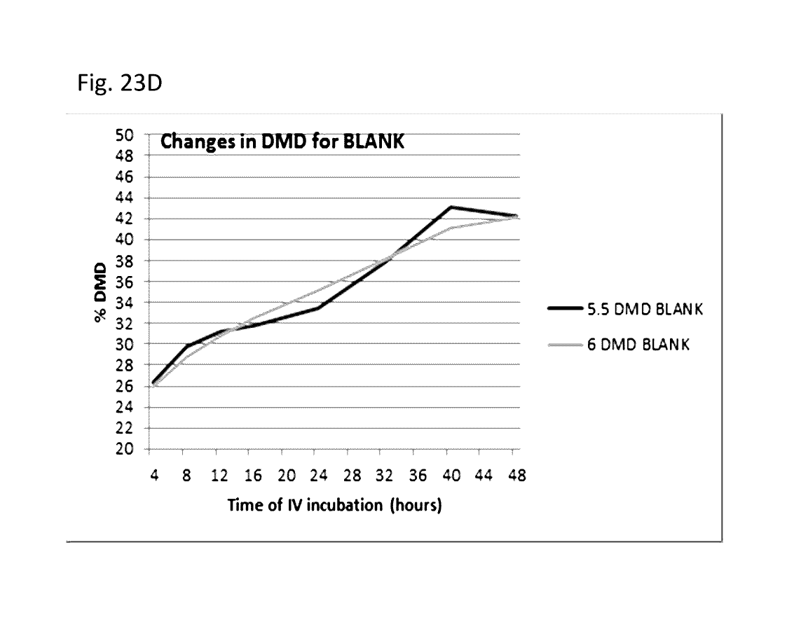

The changes in DMD over the 48 hours incubation in the 8 treatment groups are shown in FIGS. 23 A-D. The DMD content at each four-hour time point for each treatment group and the asymptotes are plotted for each treatment group, with FIG. 23 A showing the percent DMD and DMD change over time for TP25 at pH 5.5 and 6, FIG. 23 B showing the percent DMD and DMD change over time for TP50 at pH 5.5 and 6, FIG. 23 C showing the percent DMD and DMD change over time for TP75 at pH 5.5 and 6, and FIG. 23 D showing the percent DMD and DMD change over time for Blank at pH 5.5 and 6.

There was a significant difference in the shape of the DMD curve for all the treatment groups and not the blank as shown in Table 15. The difference was less for the pH 5.5 groups compared to the pH 6.0 groups.

The DMD percent values for all groups at 48 hours are shown in Table 16. The maximum at 48 hours for the TP75 group is significantly higher (p<0.05) than the other treatment groups at both pH 5.5 and pH 6.0. The TP75 group is more than 2 units higher than all other groups (Table 16), which is biologically relevant.

TABLE 16

| | 25 | 50 | 75 | Blank |
|---|---|---|---|---|
| pH 5.5 | 42.62 | 44.03 | 45.79 | 42.22 |
| pH 6.0 | 42.71 | 43.65 | 45.65 | 42.15 |

In summary, Examples 5 and 6 show that addition of TP to an in vitro ruminal incubation system shows a trend of pH increase and a significant increase in DMD content. This effect is especially observed in the TP75 group, indicating that TP, especially at the TP75 dosage level, could have an antimicrobial effect in the rumen.

### Example 7

### Elemental Analysis of Clay

A sample of Clay was analyzed for its elemental composition. The list of elements and their concentration in the sample are shown in Table 17.

TABLE 17
Nutrient Analysis of Clay

| | List of | | |
|---|---|---|---|
| | elements | Concentration % | PPM |
| 1 | Silica dioxide | 63.97 | |
| 2 | Aluminum | 16.22 | |
| 3 | Iron (FeO) | 4.95 | |
| 4 | Sulfur(Sulfide) | 3.45 | |
| 5 | Magnesium | 2.39 | |
| | oxide | | |
| 6 | Potassium | 2.09 | |
| 7 | Ferric oxide | 1.58 | |
| | (Fe₂O₃) | | |
| 8 | Magnesium | 0.94 | |
| 9 | Titanium | 0.65 | |
| | dioxide | | |
| 10 | Phosphorous | 0.14 | |
| 11 | Sodium oxide | 0.13 | |
| | (Na₂O) | | |
| 12 | Calcium | 0.1 | |
| 13 | Sulfur (sulfate) | 0.07 | |
| 14 | Gold | 0.025 | |
| 15 | Chromium | 0.01 | |
| | oxide | | |
| 16 | Sodium | 0.01 | |
| 17 | Tin | 0.01 | |
| 18 | Titanium | 0.01 | |
| 19 | Nitrogen | 0.01 | |
| 20 | Manganese | 0.005 | |
| | oxide | | |
| 21 | Copper | 0.005 | |
| 22 | Fluorine | | 361 |
| 23 | Zinc | | 142 |
| 24 | Manganese | | 120 |
| 25 | Zirconium | | 110 |
| 26 | Tellurium | | 95 |
| 27 | Rubidium | | 70 |
| 28 | Chromium | | 54 |
| 29 | Barium | | 40 |
| 30 | Vanadium | | 33 |
| 31 | Strontium | | 30 |
| 32 | Niobium | | 15 |
| 33 | Nickel | | 15 |
| 34 | Cobalt | | 14 |
| 35 | Lead | | 12 |
| 36 | Arsenic | | 12 |
| 37 | Gallium | | 10 |
| 38 | Yttrium | | 10 |
| 39 | Lanthanum | | 10 |
| 40 | Thallium | | 10 |
| 41 | Bismuth | | 7 |
| 42 | Germanium | | 7 |
| 43 | Boron | | 6 |
| 44 | Molybdenum | | 6 |
| 45 | Lithium | | 4 |
| 46 | Scandium | | 3 |
| 47 | Cadmium | | 2 |
| 48 | Antimony | | 2 |
| 49 | Selenium | | 2 |
| 50 | Thorium | | 2 |
| 51 | Tungsten | | 2 |
| 52 | Mercury | | 1 |
| 53 | Silver | | 0.6 |
| 54 | Beryllium | | 0.5 |

Additionally, the iron and sulfur compound compositions in Clay were analyzed (Table 18).

TABLE 18

| Sample | | | Calculated | Fe3+ as % Total | Sulfate | Sulfide | |
|---|---|---|---|---|---|---|---|
| Wt. | FeO | Total Fe | Fe3+ | Fe | Sulfur | Sulfur | % Oxidized S |
| kg | % | % | % | % | % | % | % |
| 0.08 | 0.93 | 2.50 | 1.57 | 62.80 | 0.81 | 1.84 | 30.57 |
| 0.12 | 0.86 | 2.61 | 1.75 | 67.05 | 0.65 | 2.16 | 23.13 |
| 0.07 | 0.66 | 3.92 | 3.26 | 83.16 | 0.11 | 3.61 | 2.96 |
| 0.20 | 0.60 | 3.65 | 3.05 | 83.56 | 0.08 | 3.19 | 2.45 |
| 0.05 | 0.40 | 2.68 | 2.28 | 85.07 | 0.11 | 2.65 | 3.99 |

### Example 8

### ADG and the Level of Fe3+ in the Clay

The average daily gain (ADG) was determined in a number of experiments, wherein the dose of antimicrobial clay was altered. In these experiments, the amounts of Fe3+ was assayed to determine the correlation between iron content and ADG. The results are shown in Table 19.

These results show that ADG values may be related to the level of Fe3+ in the clay.

### Example 9

### Evaluation of Oral Administration of Products V7, V5 and V6 Having an Antimicrobial-Effective Amount of Aluminum on Growth Performance of Weanling Pigs

A trial was conducted to evaluate the effects of feeding Products V1, V5, V6, and Denagard on growth and performance of weanling pigs challenged with F18-positive enterotoxigenic E. coll. F18-positive E. coli cause post-weaning diarrhea, also characterized by dehydration, lethargy, and wasting, often resulting in a high mortality rate. The treatments were as described in Table 20.

The elemental analysis of the antimicrobial clays, are provided in Tables 21-23 below.

TABLE 21

| | Clay1 | | | | |
|---|---|---|---|---|---|
| Analysis | Batch #1 | Batch #2 | Batch #3 | Batch #4 | Batch #5 |
| Analysis results from ALS | | | | | |
| Minerals | | | | | |
| Total Fe, % | 2.50 | 2.61 | 3.92 | 3.65 | 2.68 |
| Fe2+, % | 0.93 | 0.86 | 0.66 | 0.60 | 0.40 |
| Calcuated Fe3+, % | 1.57 | 1.75 | 3.26 | 3.05 | 2.28 |
| Sulfate S, % | 0.81 | 0.65 | 0.11 | 0.08 | 0.11 |
| Sulphide S, % | 1.84 | 2.16 | 3.61 | 3.19 | 2.65 |
| Calculated total S, % | 2.65 | 2.81 | 3.72 | 3.27 | 2.76 |
| Analysis Results from Eurofins | | | | | |
| Aluminum, % | 3 | 3.5 | 4.4 | | 5 |
| Antimony | <0.5 | <0.5 | <0.5 | | <0.5 |
| Arsenic | 8 | 9 | 0.7 | | 8 |
| Barium | 2000 | 1000 | 300 | | 1000 |
| Beryllium | <0.5 | <0.5 | <0.5 | | 0.7 |
| Bismuth | <0.5 | <0.5 | <0.5 | | <0.5 |
| Boron | <0.5 | <0.5 | <0.5 | | 0.8 |
| Cadmium | <0.5 | <0.5 | <0.5 | | <0.5 |
| Calcium, % | 0.4 | 0.5 | <0.00005 | | 2 |
| Chromium | 7 | 20 | 50 | | 20 |
| Cobalt | 7 | 8 | 10 | | 10 |
| Copper, % | 0.001 | 0.003 | 0.007 | | 0.001 |
| Fluorine | 48.8 | 50 | <5 | | 65.3 |
| Gallium | 10 | 10 | 20 | | 20 |
| Germanium | <0.5 | <0.5 | <0.5 | | <0.5 |
| Gold, % | <0.00005 | <0.00005 | <0.00005 | | <0.00005 |
| Iron, % | 2 | 2 | 3 | | 2 |
| Lanthanum | 4 | 4 | 5 | | 10 |
| Lead | 20 | 20 | 10 | | 5 |
| Lithium | 10 | 10 | 2 | | 10 |
| Magnesium, % | 0.3 | 0.3 | 0.1 | | 0.7 |
| Manganese | 100 | 100 | 200 | | 600 |
| Mercury | <0.5 | <0.5 | <0.5 | | <0.5 |
| Molybdenum | 3 | 3 | 3 | | 4 |
| Nickel | 6 | 10 | 30 | | 8 |
| Niobium | <0.5 | <0.5 | <0.5 | | <0.5 |
| Phosphorus, % | <0.02 | 0.03 | 0.01 | | 0.03 |
| Potassium, % | 0.3 | 0.3 | 0.4 | | 0.6 |
| Rubidium | 20 | 20 | 20 | | 30 |
| Scandium | 3 | 3 | 4 | | 6 |
| Selenium | 0.9 | 1 | 2 | | 0.9 |
| Silver | <0.5 | <0.5 | <0.5 | | <0.5 |
| Sodium, % | 0.1 | 0.1 | 0.2 | | 0.1 |
| Strontium | 100 | 100 | 100 | | 100 |
| Tellurium | <0.5 | <0.5 | <0.5 | | <0.5 |
| Thallium | <0.5 | <0.5 | <0.5 | | <0.5 |
| Thorium | <0.5 | <0.5 | <0.5 | | <0.5 |
| Tin, % | <0.00005 | <0.00005 | <0.00005 | | <0.00005 |
| Titanium, % | 0.0009 | 0.002 | 0.006 | | 0.009 |
| Tungsten | <0.5 | <0.5 | <0.5 | | <0.5 |
| Vanadium | 20 | 20 | 40 | | 50 |
| Yttrium | <0.5 | 3 | 2 | | 5 |
| Zinc | <50 | <50 | <50 | | 60 |
| Zirconium | 0.7 | 1 | 2 | | 2 |
| Silica, % | 63.9 | 67.1 | 64.8 | | 62.4 |
| Nitrogen, % | 0.03 | 0.06 | 0.02 | | 0.1 |
| Analysis results from Colorado | | | | | |
| School of Mines | | | | | |
| Quartz, % | 53.64 | 58.08 | 58.22 | | 48.85 |
| Kaolinite/Muscovite, % | 35.03 | 31.13 | 28.46 | | 31.69 |
| Carbonates, % | 0.28 | 0.24 | 1.80 | | 5.75 |
| Feldspar, % | 3.93 | 3.86 | 4.05 | | 6.38 |
| Biotite/Chlorite, % | 0.51 | 0.55 | 0.07 | | 0.58 |
| Tourmaline, % | 0.98 | 1.16 | 0.19 | | 1.52 |
| Pyrite, % | 2.57 | 2.84 | 5.97 | | 3.72 |
| Fe-oxides, % | 0.16 | 0.19 | 0.13 | | 0.08 |
| Fe-Aluminosilicate, % | | | | | |
| Ca—Fe Aluminosilicate, % | | | | | |
| Sphalerite, % | tr | tr | tr | | tr |
| Rutile/Anatase, % | 0.77 | 0.61 | 0.63 | | 0.55 |
| Apatite, % | 0.18 | 0.15 | 0.12 | | 0.22 |
| Barite, % | 1.85 | 1.14 | 0.16 | | 0.39 |
| Chamosite, % | | | | | |
| Rutile, % | | | | | |
| Other Minerals, % | 0.10 | 0.05 | 0.17 | | 0.26 |
| Others, % | tr | tr | tr | | tr |
| Particle size distribution, | | | | | |
| Mass % | | | | | |
| <10 um | 8.06 | 7.22 | 10.41 | | 9.32 |
| 10-20 um | 22.28 | 19.37 | 23.41 | | 29.12 |
| 20-30 um | 18.41 | 16.92 | 16.48 | | 25.80 |
| 30-40 um | 14.11 | 12.64 | 11.49 | | 17.32 |
| 40-50 um | 9.70 | 10.33 | 8.78 | | 10.68 |
| 50-75 um | 14.80 | 18.20 | 15.03 | | 7.28 |
| 75-100 um | 5.93 | 8.71 | 8.02 | | 0.47 |
| 100-125 um | 3.32 | 3.84 | 4.40 | | 0.00 |
| 125-150 um | 1.70 | 1.61 | 0.79 | | 0.00 |
| 150-175 um | 0.00 | 0.00 | 0.00 | | 0.00 |
| 175-200 um | 1.48 | 1.16 | 1.19 | | 0.00 |
| 200-225 um | 0.19 | 0.00 | 0.00 | | 0.00 |

Unit of measure is ppm unless otherwise indicated.
tr = <0.05%

TABLE 22

| Analysis | Clay2 | Clay3 | Clay4 |
|---|---|---|---|
| Analysis results from ALS | | | |
| Minerals | | | |
| Total Fe, % | 3.37 | 3.38 | 2.96 |
| Fe2+, % | 0.80 | 0.93 | 0.86 |
| Calcuated Fe3+, % | 2.57 | 2.45 | 2.10 |
| Sulfate S, % | 0.02 | 0.01 | 0.02 |
| Sulphide S, % | 0.14 | 0.16 | 0.16 |
| Calculated total S, % | 0.16 | 0.17 | 0.18 |
| Analysis Results from Eurofins | | | |
| Aluminum, % | 7.3 | 7.1 | 0.9 |
| Antimony | <0.5 | <0.5 | <0.5 |
| Arsenic | 2.3 | 2.9 | 1 |
| Barium | 72 | 69 | 40 |
| Beryllium | 1.7 | 1.5 | 0.6 |
| Bismuth | <0.5 | <0.5 | <0.5 |
| Boron | 2.2 | 3.6 | 2 |
| Cadmium | <0.5 | <0.5 | <0.5 |
| Calcium, % | 2.6 | 2 | 0.09 |
| Chromium | 2.5 | 4.7 | <0.5 |
| Cobalt | 1.4 | 1.8 | 0.7 |
| Copper, % | 0.00021 | 0.00018 | 0.0001 |
| Fluorine | 8.98 | 20.5 | 7.03 |
| Gallium | 35 | 33 | 20 |
| Germanium | <0.5 | <0.5 | <0.5 |
| Gold, % | <0.00005 | <0.00005 | <0.00005 |
| Iron, % | 2.1 | 2.2 | 0.8 |
| Lanthanum | 100 | 100 | 10 |
| Lead | 2.1 | 10 | 8 |
| Lithium | 14 | 14 | 10 |
| Magnesium, % | 1.7 | 1.3 | 0.2 |
| Manganese | 290 | 240 | 100 |
| Mercury | <0.5 | <0.5 | <0.5 |
| Molybdenum | <0.5 | <0.5 | <0.5 |
| Nickel | 3.5 | 3.6 | 0.9 |
| Niobium | 2.1 | 0.99 | 60 |
| Phosphorus, % | 0.014 | 160 | 0.004 |
| Potassium, % | 0.12 | 0.15 | 0.06 |
| Rubidium | 5.2 | 8.4 | 2 |
| Scandium | 4.8 | 3.8 | 2 |
| Selenium | 0.91 | 1 | <0.5 |
| Silver | <0.5 | <0.5 | 0.8 |
| Sodium, % | 0.048 | 0.012 | 0.09 |
| Strontium | 1300 | 970 | 400 |
| Tellurium | <0.5 | <0.5 | <0.5 |
| Thallium | <0.5 | <0.5 | <0.5 |
| Thorium | 14 | 13 | 2 |
| Tin, % | <0.0005 | <0.0005 | 0.0002 |
| Titanium, % | 0.1 | 0.13 | 0.01 |
| Tungsten | <0.5 | <0.5 | <0.5 |
| Vanadium | <13 | <16 | 10 |
| Yttrium | 28 | 28 | 6 |
| Zinc | 61 | 83 | 40 |
| Zirconium | 120 | 130 | 200 |
| Silica, % | 51 | 55.8 | 57.7 |
| Nitrogen, % | 0.04 | 0.03 | 0.02 |
| Analysis results from Colorado | | | |
| School of Mines | | | |
| Quartz, % | 0.38 | 5.42 | 3.27 |
| Kaolinite/Muscovite, % | 89.48 | 86.12 | 87.99 |
| Carbonates, % | 1.23 | 0.94 | 1 |
| Feldspar, % | 4.36 | 4.36 | 4.46 |
| Biotite/Chlorite, % | 0.02 | 0.09 | 0.08 |
| Tourmaline, % | tr | tr | tr |
| Pyrite, % | 0.2 | 0.11 | 0.13 |
| Fe-oxides, % | tr | tr | 0.01 |
| Fe-Aluminosilicate, % | | | |
| Ca—Fe Aluminosilicate, % | | | |
| Sphalerite, % | tr | tr | 0.01 |
| Rutile/Anatase, % | 0.01 | 0.04 | 0.05 |
| Apatite, % | 0.01 | 0.03 | tr |
| Barite, % | tr | tr | tr |
| Chamosite, % | | | |
| Rutile, % | | | |
| Other Minerals, % | tr | 0.01 | 0.01 |
| Others, % | tr | tr | tr |
| Particle size distribution, Mass % | | | |
| <6 um | 8.06 | 7.22 | 10.41 |
| 6-10 um | 26.93 | 1.16 | 6.87 |
| 10-15 um | 28.41 | 2.59 | 11.82 |
| 15-18 um | 9.3 | 1.48 | 6.64 |
| 18-22 um | 5.69 | 1.91 | 7.85 |
| 22-26 um | 1.86 | 2.01 | 6.84 |
| 26-32 um | 0.62 | 3.02 | 8.31 |
| 32-38 um | 0.07 | 4.3 | 8.35 |
| 38-46 um | 0.01 | 6.04 | 8.04 |
| 46-55 um | 0 | 7.66 | 8.55 |
| 55-66 um | 0 | 8.52 | 8 |
| 66-79 um | 0 | 9.23 | 5.63 |
| 79-95 um | 0 | 7.88 | 4.61 |
| 95-115 um | 0 | 7.93 | 1.33 |
| 115-138 um | 0 | 7 | 0.83 |
| 138-199 um | 0 | 6.93 | 0 |
| 166-199 um | 0 | 5.91 | 0 |
| 199-239 um | 0 | 4.76 | 0 |
| 139-288 um | 0 | 3.52 | 0 |
| 288-346 um | 0 | 2.37 | 0 |
| 346-416 um | 0 | 1.53 | 0 |
| 416-500 um | 0 | 1.92 | 0 |
| >500 um | 0 | 1.43 | 0 |

Unit of measure is ppm unless otherwise indicated.
tr = <0.05%

TABLE 23

| Analysis | Clay5-145 | Clay5-20 |
|---|---|---|
| Analysis results from ALS Minerals | | |
| Total Fe, % | 3.77 | 3.38 |
| Fe2+, % | 0.84 | 0.51 |
| Calcuated Fe3+, % | 2.93 | 2.87 |
| Sulfate S, % | <0.01 | <0.01 |
| Sulphide S, % | 0.01 | 0.01 |
| Calculated total S, % | <0.02 | <0.02 |
| Analysis Results from Eurofins | | |
| Aluminum, % | 3.9 | 3.9 |
| Antimony | <0.5 | <0.5 |
| Arsenic | 10 | 10 |
| Barium | 60 | 70 |
| Beryllium | 0.6 | 0.7 |
| Bismuth | <0.5 | <0.5 |
| Boron | 50 | 60 |
| Cadmium | <0.5 | <0.5 |
| Calcium, % | 1 | 0.4 |
| Chromium | 30 | 9 |
| Cobalt | 7 | 6 |
| Copper, % | 0.001 | 0.0007 |
| Fluorine | <5 | <5 |
| Gallium | 10 | 9 |
| Germanium | <0.5 | <0.5 |
| Gold, % | <0.00008 | <0.00008 |
| Iron, % | 3 | 2 |
| Lanthanum | 20 | 30 |
| Lead | 5 | 5 |
| Lithium | 8 | 9 |
| Magnesium, % | 0.2 | 0.2 |
| Manganese | 500 | 500 |
| Mercury | <0.5 | <0.5 |
| Molybdenum | 0.5 | <0.5 |
| Nickel | 10 | 4 |
| Niobium | <0.5 | <0.5 |
| Phosphorus, % | 0.02 | 0.02 |
| Potassium, % | 0.5 | 0.5 |
| Rubidium | 7 | 6 |
| Scandium | 9 | 8 |
| Selenium | <0.5 | <0.5 |
| Silver | <0.5 | 0.6 |
| Sodium, % | 0.01 | 0.009 |
| Strontium | 40 | 40 |
| Tellurium | <0.5 | <0.5 |
| Thallium | <0.5 | <0.5 |
| Thorium | 1 | 1 |
| Tin, % | <0.0004 | <0.0004 |
| Titanium, % | 0.002 | 20 |
| Tungsten | <0.5 | <0.5 |
| Vanadium | 40 | 40 |
| Yttrium | 20 | 20 |
| Zinc | 50 | 40 |
| Zirconium | 2 | 0.9 |
| Silica, % | 61 | 64.7 |
| Nitrogen, % | 0.02 | <0.02 |
| Analysis results from Colorado School of | | |
| Mines | | |
| Quartz, % | 52.09 | 54.46 |
| Kaolinite/Muscovite, % | 12.6 | 18.15 |
| Carbonates, % | | |
| Feldspar, % | 21.35 | 16.41 |
| Biotite/Chlorite, % | 0.07 | 0.06 |
| Tourmaline, % | | |
| Pyrite, % | 0.01 | 0.01 |
| Fe-oxides, % | 3.74 | 2.87 |
| Fe-Aluminosilicate, % | 5.39 | 4.22 |
| Ca—Fe Aluminosilicate, % | 3.47 | 3.19 |
| Sphalerite, % | | |
| Rutile/Anatase, % | 0.57 | 0.32 |
| Apatite, % | | |
| Barite, % | | |
| Chamosite, % | 0.27 | 0.14 |
| Rutile, % | 0.57 | 0.32 |
| Other Minerals, % | 0.44 | 0.17 |
| Others, % | tr | tr |
| Particle size distribution, Mass % | | |
| 5.0-7.9 μm | 20.21 | 8.74 |
| 7.9-13 μm | 21.87 | 10.56 |
| 13-20 μm | 27.22 | 17.64 |
| 20-32 μm | 17.91 | 15.88 |
| 32-50 μm | 8.28 | 11.7 |
| 50-79 μm | 3.54 | 9.47 |
| 79-126 μm | 0.79 | 8.38 |
| 126-199 μm | 0.18 | 7.3 |
| 199-315 μm | 0 | 6.28 |
| 315-500 μm | 0 | 2.82 |
| >500 μm | 0 | 1.22 |

Unit of measure is ppm unless otherwise indicated.
tr = <0.05%

Denagard (tiamulin) is a solution containing 12.5% tiamulin hydrogen fumarate (w/v) in an aqueous solution. The active ingredient, tiamulin, chemically is 14-desoxy-14-[(2-diethylaminoethyl) mercaptoacetoxy] mutilin hydrogen fumarate, a semi-synthetic diterpene antibiotic.

In all, 40 mixed-sex weanling pigs were used. The pigs were blocked by body weight at weaning into five treatment groups, with eight pigs per treatment. The treatments included (1) pigs that were fed the control diet without any treatment (control), (2) pigs that were treated with Clay1, (3) pigs that were treated with Clay5, (4) pigs that were treated with Clay6, and (5) pigs that were treated with Denagard. All pigs were challenged with F18-positive enterotoxigenic E. coli (E. coli F18).

On days 0-6, all pigs were fed their respective experimental diets to adapt the pigs to the diets. The experimental diets comprised a basal diet as described in Table 24, with the various products added to the diet at a rate as disclosed in Table 20. The basal diet did not contain medications, added Zn or Cu (except for Zn and Cu in VTM). All pigs were challenged by inoculating with 5 ml of E. coli F18 (10⁹ CFU) on day 6 and day 7. On day 10, the trial was ended and the following parameters were assessed: pre-challenge and post-challenge average daily gain (ADG), average and 72 hr post-challenge fecal score, % diarrhea frequency, and E. coli count in feces. Pro-inflammatory cytokines were also measured.

TABLE 24

| | lbs/ton |
|---|---|
| Ingredients | |
| Corn 600 14% moisture | 864.45 |
| Soybean 46% | 350.00 |
| Hamlet HP300 | 150.00 |
| betaGRO | 6.00 |
| Menhaden SS - Fish Meal | 96.00 |
| Lysine 98.5 | 5.60 |
| Methionine 99% DL | 3.05 |
| Threonine 98.5% | 2.20 |
| 21% Monocal | 13.50 |
| Limestone | 10.20 |
| Salt | 11.00 |
| Choline Chloride 60% | 1.00 |
| Nursery VTM 3# | 3.00 |
| Whey dried | 417.00 |
| Corn oil | 67.00 |
| Total | 2000.00 |
| Analyzed Nutrients Composition | |
| ME, kcal/lb | 1529.85 |
| Crude Protein, % | 22.41 |
| TID Lysine, % | 1.38 |
| Avail Phos, % | 0.50 |
| Lactose | 15.01 |

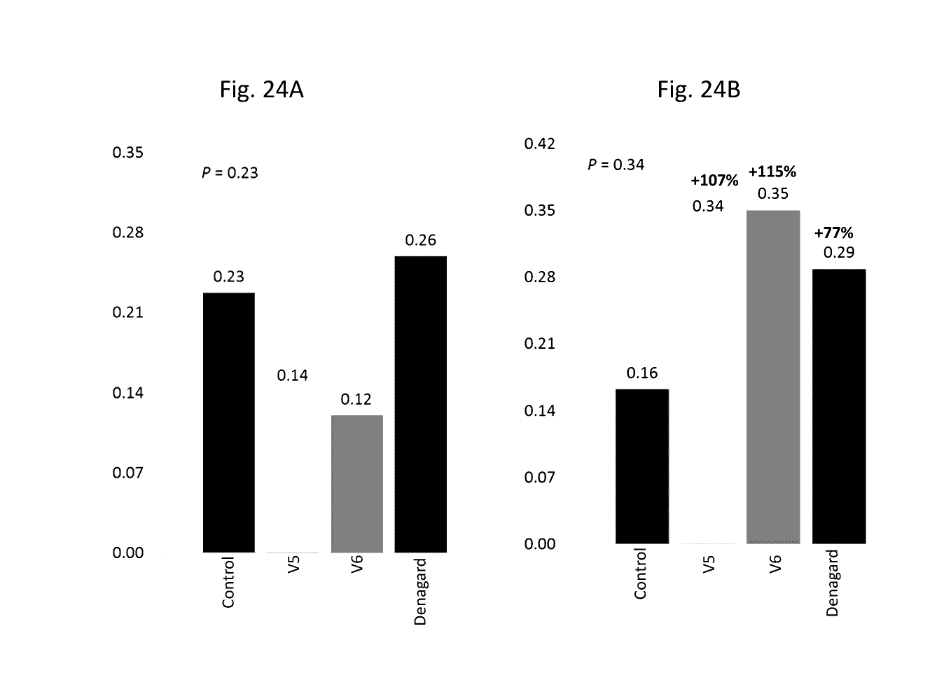

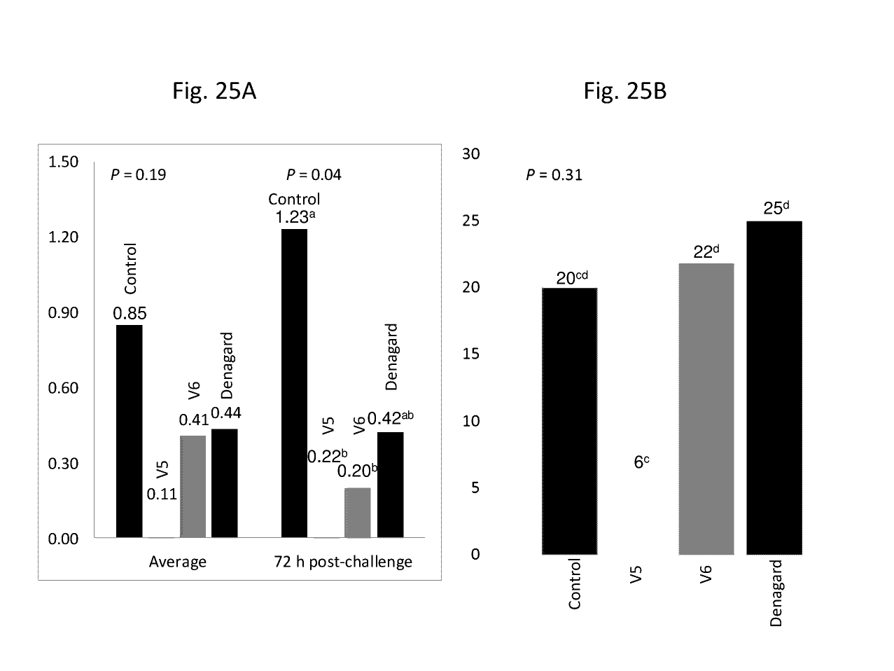

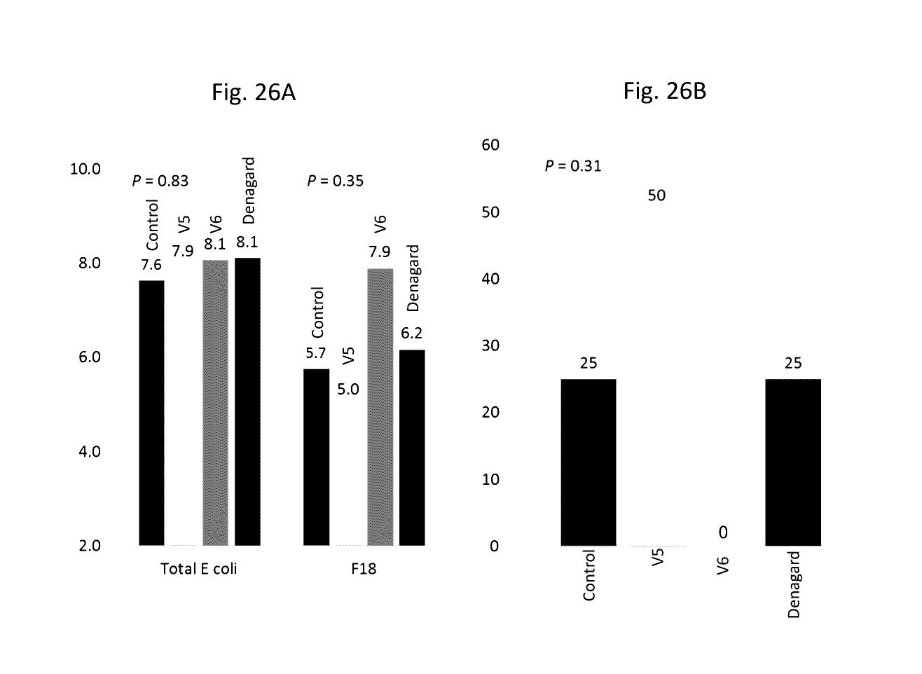

In all, feeding Products V5 and V6 to weanling pigs challenged with E. coli F18 numerically improved ADG post-challenge (FIG. 24). Administering Products V5 and V6 also significantly reduced the fecal score (FIG. 25A). Additionally, Clay5 significantly reduced the frequency of diarrhea (FIG. 25B). Although the total E. coli and E. coli F18 count in pig feces was not significantly changed with the various treatments (FIG. 26A), the number of pigs with undetectable E. coli F18 was significantly higher in pigs administered Clay5 (FIG. 26B). Further, feeding pigs Products V5 and V6 tended to reduce serum IL-8 on 4-dpi, indicating less activated immune system following the F18 challenge.

What is claimed is:

1. A method for controlling microbes, the method comprising contacting the microbes with an antimicrobial effective amount of an antimicrobial clay, wherein the clay is mined clay.

2. The method of claim 1, wherein the antimicrobial clay is clay mined in the Crater Lake region of the Cascade Mountains of Oregon.

3. The method of claim 1, wherein the antimicrobial clay comprises an antimicrobial effective amount of a reducing agent.

4. The method of claim 1, wherein the antimicrobial clay comprises an antimicrobial effective amount of aluminum.

5. The method of claim 1, wherein the antimicrobial clay comprises about 1% to about 15% aluminum.

6. The method of claim 1, wherein the antimicrobial clay comprises about 2% to about 5% aluminum.

7. The method of claim 1, wherein the antimicrobial clay comprises about 3% to about 10% pyrite.

8. The method of claim 1, wherein the antimicrobial clay comprises about 1% to about 5% Fe³⁺.

9. The method of claim 1, wherein the antimicrobial clay comprises about 3% to about 10% pyrite, and about 1% to about 5% Fe³⁺.

10. The method of claim 1, wherein the antimicrobial clay comprises about 3% to about 10% pyrite, about 1% to about 5% Fe³⁺, and about 3% to about 15% aluminum.

11. The method of claim 1, wherein the antimicrobial clay is naturally mined, and the level of reducing agent in the clay is adjusted to provide antimicrobial effective amounts of the reducing agent.

12. The method of claim 1, wherein the average particle size of the antimicrobial clay is less than about 500 microns in diameter.

13. The method of claim 1, wherein the average particle size of the antimicrobial clay is less than about 300 microns in diameter.

14. The method of claim 1, wherein the average particle size of the antimicrobial clay is between about 20 microns and about 200 microns in diameter.

15. The method of claim 1, wherein the average particle size of the antimicrobial clay is between about 25 microns and about 150 microns in diameter.

16. The method of claim 1, further comprising administering the antimicrobial clay to an animal to inhibit the growth of bacteria in the animal.

17. The method of claim 16, wherein the bacteria are selected from the group consisting of Clostridium perfringens, Aeromonas hydrophila, Yersinia enterocolitica, Vibrio spp., Leptospira spp., Mycobacterium ulcerans, Listeria spp., pathogenic strains of E. coli, Pseudomonas spp., Staphylococcus spp., Streptococcus sp., Clostridia, and M. marinum, Lawsonia, Salmonella, Campylobacter, Enterococcus, Liver abscess bacteria.

18. The method of claim 16, wherein the administering is oral administration.

19. The method of claim 19, wherein the antimicrobial clay is formulated in a feed composition for oral administration to the animal.

20. The method of claim 20, wherein the amount of antimicrobial clay in a feed composition ranges from about 0.1% to about 0.5% of the feed composition.

21. The method of claim 1, wherein the antimicrobial clay is administered to an animal at a rate of about 3 to about 10 grams per animal per day.

22. The method of claim 1, wherein the antimicrobial clay is administered to an animal at a rate of about 0.3 to about 4 grams per animal per day.

23. The method of claim 1, wherein the antimicrobial clay is administered to an animal at a rate of about 0.05 to about 5 grams/lb body weight/day.

24. The method of claim 1, wherein the antimicrobial clay is administered to an animal at a rate of about 0.025 to about 0.2 grams/lb body weight/day.

25. The method of claim 17, wherein the antimicrobial clay is administered to a pig to control enterotoxigenic E. coli in the pig.

26. The method of claim 17, wherein the antimicrobial clay is administered to a chicken to control necrotic enteritis in the chicken.

27. The method of claim 17, wherein the antimicrobial clay is administered to a pig to control influenza in the pig.

28. The method of claim 17, wherein the antimicrobial clay is administered to a pig to control scouring in the pig.

29. The method of claim 1, the method comprising administering the antimicrobial clay to an animal to improve growth performance of the animal.

30. The method of claim 17, wherein the antimicrobial clay is administered at least once daily.

31. The method of claim 1, the method comprising contacting an animal's environment with the antimicrobial clay to control pathogenic microbes in the animal's environment.

32. The method of claim 1, the method comprising contacting a fermenting mixture with the antimicrobial clay to control bacteria during fermentation.

33. A method for treating a microbial infection in an animal, the method comprising administering a feed composition to the animal, wherein the composition comprises an antimicrobial effective amount of a mined antimicrobial clay, and wherein the clay is mined in the Crater Lake region of the Cascade Mountains of Oregon.

34. The method of claim 34, wherein the amount of antimicrobial clay in a feed composition ranges from about 0.1% to about 0.5%.

35. The method of claim 34, wherein the composition is administered at least once daily.

36. The method of claim 34, wherein the microbial infection is selected from enterotoxigenic E. coli in the pig, necrotic enteritis in the chicken, influenza in the pig, or scouring in the pig.

37. A method of improving growth performance of an animal, the method comprising orally administering a feed composition to the animal, wherein the composition comprises an antimicrobial effective amount of an antimicrobial clay, and wherein the clay is mined in the Crater Lake region of the Cascade Mountains of Oregon.

38. The method of claim 38, wherein the amount of antimicrobial clay in the feed composition ranges from about 0.1% to about 0.5%.

39. The method of claim 38, wherein the composition is administered at least once daily.

40. A method of controlling pathogenic microbes in an animal's environment, the method comprising contacting the animal's environment with an antimicrobial effective amount of an antimicrobial clay, wherein the clay is mined in the Crater Lake region of the Cascade Mountains of Oregon.

41. A method for controlling bacteria during fermentation, the method comprising contacting a fermentation mixture with an antimicrobial effective amount of an antimicrobial clay, wherein the clay is mined in the Crater Lake region of the Cascade Mountains of Oregon.

42. An antimicrobial feed composition comprising an antimicrobial effective amount of an antimicrobial clay, wherein the clay is mined clay.

43. The composition of claim 42, wherein the antimicrobial clay is clay mined in the Crater Lake region of the Cascade Mountains of Oregon.

44. The composition of claim 42, wherein the amount of antimicrobial clay in a feed composition ranges from about 0.1% to about 0.5%.

45. The composition of claim 42, wherein the antimicrobial clay comprises about 1% to about 15% aluminum.

46. The composition of claim 42, wherein the antimicrobial clay comprises about 2% to about 5% aluminum.

47. The composition of claim 42, wherein the antimicrobial clay comprises about 3% to about 10% pyrite.

48. The composition of claim 42, wherein the antimicrobial clay comprises about 1% to about 5% Fe³⁺.

49. The composition of claim 42, wherein the antimicrobial clay comprises about 3% to about 10% pyrite, and about 1% to about 5% Fe³⁺.

50. The composition of claim 42, wherein the antimicrobial clay comprises about 3% to about 10% pyrite, about 1% to about 5% Fe³⁺, and about 3% to about 15% aluminum.

51. A method of treating a microbial infection in an animal, the method comprising:
a. providing an antimicrobial clay, wherein the clay is mined clay;
b. combining an antimicrobial effective amount of the antimicrobial clay with an animal feed composition to prepare an antimicrobial feed composition; and
c. feeding the antimicrobial feed composition to the animal to treat the microbial infection.

52. The composition of claim 51, wherein the antimicrobial clay is clay mined in the Crater Lake region of the Cascade Mountains of Oregon.

53. The composition of claim 51, wherein the antimicrobial effective amount of antimicrobial clay is combined with the animal feed at the rate of about 0.1% to about 0.5% wt/wt of the antimicrobial feed composition.

54. The composition of claim 51, wherein the antimicrobial clay comprises about 3% to about 10% pyrite, and about 1% to about 5% Fe³⁺.

55. The composition of claim 51, wherein the antimicrobial clay comprises about 3% to about 10% pyrite, about 1% to about 5% Fe³⁺, and about 3% to about 15% aluminum.

56. A method of improving growth performance of an animal, the method comprising:
a. providing an antimicrobial clay, wherein the clay is mined clay;
b. combining an antimicrobial effective amount of the antimicrobial clay with an animal feed composition to prepare an antimicrobial feed composition; and
c. feeding the antimicrobial feed composition to the animal to improve growth performance of the animal.

57. The composition of claim 56, wherein the antimicrobial clay is clay mined in the Crater Lake region of the Cascade Mountains of Oregon.

58. The composition of claim 56, wherein the antimicrobial effective amount of antimicrobial clay is combined with the animal feed at the rate of about 0.1% to about 0.5% wt/wt of the antimicrobial feed composition.

59. The composition of claim 56, wherein the antimicrobial clay comprises about 3% to about 10% pyrite, and about 1% to about 5% Fe³⁺.

60. The composition of claim 56, wherein the antimicrobial clay comprises about 3% to about 10% pyrite, about 1% to about 5% Fe³⁺, and about 3% to about 15% aluminum.

61. The method of claim 53, wherein the antimicrobial feed composition is fed to the animal at least once daily.
